(19) United States
(12) Patent Application Publication
(10) Pub. No.: US 2023/0106635 A1
(43) Pub. Date: Apr. 6, 2023

(54) HYPOALLERGENIC RECOMBINANT MILK PROTEINS AND COMPOSITIONS COMPRISING THE SAME

(71) Applicant: Perfect Day, Inc., Berkeley, CA (US)

(72) Inventors: Vaibhav Bhatt, Bangalore (IN); Louis Clark, San Francisco, CA (US); Timothy Geistlinger, Oakland, CA (US); Janine Lin, Davis, CA (US)

(21) Appl. No.: 17/904,571

(22) PCT Filed: Feb. 19, 2021
(86) PCT No.: PCT/US21/18899

§ 371 Date: Aug. 18, 2022

Related U.S. Application Data
(60) Provisional application No. 62/978,726, filed on Feb. 19, 2020.

(51) Int. Cl. A23L 33/195; A23J 1/00; A23J 3/20; C07K 14/47; C12P 21/02

(52) U.S. Cl. CPC A23L 33/195; A23J 1/008; A23J 3/20; C07K 14/4717; C12P 21/02; A23V 2002/00

(57) ABSTRACT

Provided is a recombinant milk protein having an attenuated or essentially eliminated allergenicity, compositions comprising the recombinant milk protein, and methods for producing the recombinant milk and compositions.

### FIELD OF THE INVENTION

The present invention relates generally to recombinant milk proteins, compositions comprising such recombinant milk proteins, and to methods for producing such recombinant milk proteins and compositions. In particular, the present invention relates to recombinant milk proteins having attenuated or essentially eliminated allergenicity.

### BACKGROUND OF THE INVENTION

Milk is a popular source of nutrition. It comprises high-quality protein, essential minerals, and vitamins. In addition, milk comprises proteins with advantageous functional characteristics that permit production of a wide variety of derivative products (e.g., yogurt, cheese, cream, butter), and that are useful in industrial applications (e.g., production of polymers, therapeutics, household products).

Although nutritious and versatile, milk and dairy products cannot be consumed by an increasing number of people in which it elicits allergenic reactions. Allergenicity is a particular problem for the whey protein β-lactoglobulin. The allergenicity of β-lactoglobulin may be due to the presence of T-cell and B-cell antigenic epitopes [e.g., amino acid sequences, or secondary or tertiary structures that are recognized by T-cell surface antigens e.g., major histocompatibility complex (MHC) class I and class II proteins] and B-cell produced immunoglobulins [e.g., IgE]), combined with an absence of solvent exposed protease recognition or cleavage sequences, and a relative stability of the protein to denaturation in the acidic environment of the stomach. As a result, non-digested β-lactoglobulin comprising antigenic epitopes can reach sites of active immune sampling in the gastro-intestinal mucosa and initiate an allergenic reaction.

The allergenicity of milk proteins is a significant motivation behind efforts to develop alternatives to milk and dairy products (e.g., plant-based and nut-based milk-/dairy-like food products). However, these efforts have to date fallen short on matching flavor and nutritional profile of milk and dairy products, and on recreating proteins that have identical or similar nutritive contents and functionalities as milk proteins.

Therefore, there exists a need for hypoallergenic milk proteins with attenuated or essentially eliminated allergenicity, and compositions (e.g., food products) that comprise such milk proteins.

### INCORPORATION BY REFERENCE

All publications, patents, patent applications, sequences, database entries, scientific publications, and other references mentioned herein are incorporated by reference in their entireties to the same extent as if each individual publication, patent, patent application, sequence, database entry, scientific publication, or other reference was specifically and individually indicated to be incorporated by reference. To the extent the material incorporated by reference contradicts or is inconsistent with the present disclosure, the present disclosure, including definitions, will supersede any such material.

### SUMMARY OF THE INVENTION

In one aspect, provided herein is a recombinant milk protein that comprises a modification compared to a corresponding native milk protein that attenuates or essentially eliminates allergenicity of the recombinant milk protein compared to the corresponding native milk protein. The modification can eliminate an allergenic epitope from the recombinant milk protein that is comprised in the corresponding native milk protein, eliminate a post-translational modification (PTM; e.g., an O-glycosylation) in the recombinant milk protein that is comprised in the corresponding native milk protein, decrease stability of a protein structure of the recombinant milk protein at an acidic pH compared to stability of the corresponding native milk protein, and/or create a non-native protease recognition or cleavage sequence in the recombinant milk protein. The non-native protease recognition or cleavage sequence can be a non-native recognition or cleavage sequence for a protease comprised in the gastrointestinal tract of a mammal (e.g., a human). The recombinant milk protein according to any of the above can be a recombinant β-lactoglobulin protein. The recombinant milk protein according to any of the above can be a recombinant α-lactalbumin protein.

In another aspect, provided herein is a composition that comprises a milk protein component and that has an attenuated or essentially eliminated allergenicity compared to a corresponding composition, wherein the milk protein component comprises or consists of the recombinant milk protein according to any of the above.

In other aspects, provided herein are a recombinant host cell capable of producing the recombinant milk protein according to any of the above, and a recombinant expression construct and recombinant vector useful for producing the recombinant host cell, as well as methods for obtaining the recombinant host cell, and for producing the recombinant milk protein and composition comprising the recombinant milk protein.

### BRIEF DESCRIPTION OF THE FIGURES

Example embodiments will be described and explained with additional specificity and detail through the use of the accompanying drawings in which:

FIG. 1 is a map of a base Trichoderma reesei targeting vector used for production of a recombinant milk protein having attenuated or essentially eliminated allergenicity, in accordance with representative embodiments of the present invention.

FIG. 2 is a map of base Pichia pastoris (Komagataella phaffii) targeting vector used for production of a recombinant milk protein having attenuated or essentially eliminated allergenicity, in accordance with representative embodiments of the present invention.

### DETAILED DESCRIPTION OF THE INVENTION

The terminology and description used herein is for the purpose of describing particular embodiments only, and is not intended to limit the scope of the invention. Unless defined otherwise, all technical and scientific terms used herein have the same meaning as is commonly understood by one of ordinary skill in the art to which this disclosure pertains. Further, unless otherwise required by context, singular terms shall include the plural, and plural terms shall include the singular.

### Definitions

Amino acids can be referred to herein by their single-letter codes, amino acid names, or three-letter codes. The single-letter codes, amino acid names, and three-letter codes are as follows: G—Glycine (Gly), P—Proline (Pro), A—Alanine (Ala), V—Valine (Val), L—Leucine (Leu), I—Isoleucine (Ile), M—Methionine (Met), C—Cysteine (Cys), F—Phenylalanine (Phe), Y—Tyrosine (Tyr), W—Tryptophan (Trp), H—Histidine (His), K—Lysine (Lys), R—Arginine (Arg), Q—Glutamine (Gln), N—Asparagine (Asn), E—Glutamic Acid (Glu), D—Aspartic Acid (Asp), S—Serine (Ser), T—Threonine (Thr). Amino acid residues are denoted by a first letter for the amino acid, followed by a number that specifies the position of the amino acid in a reference sequence (e.g., SEQ ID NO: 1 or 2). Amino acid substitutions are denoted by a first letter for the amino acid that is to be replaced, followed by a number that specifies the position of the amino acid to be replaced in a reference sequence (e.g., SEQ ID NO: 1 or 2), and a second letter that is to be substituted at the position in place of the amino acid that is to be replaced.

The terms “a” and “an” and “the” and similar references as used herein refer to both the singular and the plural (e.g., meaning “at least one” or “one or more”), unless otherwise indicated herein or clearly contradicted by context. For example, the term “a compound” is synonymous with the terms “at least one compound” and “one or more compounds”, and may refer to a single compound or to a plurality of compounds, including mixtures thereof.

The term “about” as used herein in conjunction with a stated numerical value or range of numerical values is meant to encompass variations of the stated numerical value or range of numerical values (i.e., denoting somewhat more or somewhat less than the stated numerical value or range of numerical values, to within a range of +10% of the stated value or range of numerical values).

The term “allergenic epitope” as used herein refers to an amino acid sequence that elicits an allergenic response in a human or other animal. Typically, such allergenic response is elicited due to binding of a T-cell surface antigen (e.g., MHC class I and II proteins) or a B-cell produced immunoglobulin (e.g., IgE) to a T-cell allergenic epitope or a B-cell antigenic epitope, respectively.

The term “and/or” as used herein refers to multiple components in combination with or exclusive of one another. For example, “x, y, and/or z” can refer to “x” alone, “y” alone, “z” alone, “x, y, and z”, “(x and y) or z”, “(x and z) or y”, “Cy and z) or x”, “x and y” alone, “x and z” alone, “y and z” alone, or “x or y or z”.

The term “at least” or “one or more” as used herein refers to one, two, three, four, five, six, seven, eight, nine, ten, at least one, at least two, at least three, at least four, at least five, at least six, at least seven, at least eight, at least nine, at least ten, or more, or all of the elements subsequently listed.

The term “B-cell antigenic epitope” as used herein refers to an amino acid sequence that is recognized (e.g., bound) by a B-cell produced immunoglobulin (e.g., IgE).

The term “casein” as used herein refers to a polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in a casein natively found in a mammal-produced milk (i.e., a casein that is native to a mammal-produced milk; e.g., a native casein). Non-limiting examples of caseins include β-casein (e.g., amino acids 16 to 224 of UniProt sequence P02666; amino acids 16 to 222 of UniProt sequence P11839 or P33048; amino acids 16 to 226 of P05814), γ-casein, κ-casein (e.g., amino acids 22 to 190 of UniProt sequence P02668; amino acids 22 to 192 of UniProt sequence P02669 or P02670; amino acids 21 to 182 of UniProt sequence P07498), α-S1-casein (e.g., amino acids 16 to 214 of UniProt sequence P02662, P04653, or P18626; amino acids 16 to 185 of UniProt sequence P47710), and α-S2-casein (e.g., amino acids 16 to 222 of UniProt sequence P02663; amino acids 16 to 223 of UniProt sequence P04654 or P33049, respectively).

The term “corresponding composition” or “corresponding food product” as used herein refers to a composition or food product, respectively, that is produced by a method that is identical to the method used to produce the composition or food product, respectively, that is compared to the “corresponding composition” or “corresponding food product”, respectively, except that the method by which the “corresponding composition” or “corresponding food product”, respectively, is produced does not comprise a step in which an allergenicity of the “corresponding composition” or “corresponding food product”, respectively, is attenuated or essentially eliminated as provided in a method provided herein.

The term “corresponding native milk protein”, “corresponding native β-lactoglobulin protein”, or “corresponding native α-lactalbumin protein” as used herein refers to a native milk protein, β-lactoglobulin protein, or α-lactalbumin protein, respectively, that is identical to a recombinant milk protein, recombinant β-lactoglobulin protein, or recombinant α-lactalbumin protein, respectively, that is compared to the “corresponding native milk protein”, “corresponding native β-lactoglobulin protein”, or “corresponding native α-lactalbumin protein”, respectively, except that it does not comprise a modification as provided in the recombinant milk protein, recombinant β-lactoglobulin protein, or recombinant α-lactalbumin protein, respectively.

The term “digestibility” as used herein refers to the rate at which an agent is degraded in a human or other animal gastrointestinal tract.

The term “essentially free of” as used herein refers to the indicated component being either not detectable in the indicated composition by common analytical methods, or to the indicated component being present in such trace amount as to not be functional. The term “functional” as used in this context refers to not contributing to properties of the composition comprising the trace amount of the indicated component, or to not having activity (e.g., chemical activity, enzymatic activity) in the indicated composition comprising the trace amount of the indicated component, or to not having health-adverse effects upon use or consumption of the composition comprising the trace amount of the indicated component.

The term “filamentous fungus” as used herein refers to any filamentous form of the subdivision Eumycota and Oomycota (as defined by Hawksworth et al., In, Ainsworth and Bisby's Dictionary of The Fungi, 8th edition, 1995, CAB International, University Press, Cambridge. UK). A filamentous fungus is distinguished from yeast by its hyphal elongation during vegetative growth. The term “filamentous fungal host cell” as used herein refers to a host cell that is obtained from a filamentous fungus.

The term “food product” as used herein refers to a composition that can be ingested by a human or an animal for dietary purposes (i.e., without ill health effects but with significant nutritional and/or caloric intake due to uptake of digested material in the gastrointestinal tract), including a domesticated animal (e.g., dog, cat), farm animal (e.g., cow, pig, horse), and wild animal (e.g., non-domesticated predatory animal). The term includes compositions that can be combined with or added to one or more other ingredients to make a food product that can be ingested by a human or an animal.

The term “fungus” as used herein refers to organisms of the phyla Ascomycotas, Basidiomycota, Zygomycota, and Chythridiomycota, Oomycota, and Glomeromycota. It is understood, however, that fungal taxonomy is continually evolving, and therefore this specific definition of the fungal kingdom may be adjusted in the future. The term “fungal host cell” as used herein refers to a host cell that is obtained from a fungus.

The term “gastrointestinal tract” as used herein comprises mouth, esophagus, stomach, small intestine, large intestine, and anus.

The term “homolog” as used herein refers to a protein that comprises an amino acid sequence that is at least 40% (e.g., at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100%) identical to a sequence of amino acids of a similar length (i.e., a length that is within +/−20% of the length of the query amino acid sequence) comprised in a reference protein, and that has a functional property that is similar to (e.g., is within 50%, within 40%, within 30%, within 20%, or within 10% of) that of the reference protein. The term includes polymorphic variants, interspecies homologs (e.g., orthologs), paralogs, and alleles of a protein.

The term “host cell” as used herein refers not only to the particular subject cell but to the progeny of such cell. Because certain modifications may occur in succeeding generations due to either mutation or environmental influences, such progeny may not, in fact, be identical to the subject cell, but are still included within the scope of the term “host cell” as used herein.

The terms “hypoallergenic” and/or “attenuated or essentially eliminated allergenicity” as used herein with reference to an agent refers to a reduction or elimination of an allergic reaction elicited in a human upon ingestion of or contact with the agent. Reduction is typically scored in comparison to a reference agent, which in the context of a recombinant milk protein provided herein (e.g., a recombinant β-lactoglobulin protein provided herein) is a corresponding native milk protein (e.g., a native β-lactoglobulin protein), and in context of a composition provided herein (e.g., a food product provided herein) is a corresponding composition (e.g., a corresponding food product). Such reduction can be an allergenicity of no more than 0%, 1%, 5%, 10%, 15%, 20%, 25%, 30%, 35%, 40%, 45%, 50%, 55%, 60%, 65%, 70%, 75%, 80%, 85%, 90%, or 95% of that of the corresponding native milk protein or corresponding composition. Allergenicity may be measured using a variety of methods known in the art, including methods provided herein.

The terms “identity” or “identical” in the context of two or more polynucleotide or polypeptide sequences as used herein refer to the nucleotide or amino acid residues that are the same when the two or more polynucleotide or polypeptide sequences, respectively, are aligned for maximum correspondence. Depending on the application, the “identity” can exist over a region of the sequences being compared (e.g., over the length of a functional domain) or over the full length of the sequences. A “region” is considered to be a continuous stretch of at least 6, 9, 14, 19, 24, 29, 34, 39, or more nucleotides, or of at least 2, 6, 10, 14, 18, 22, 26, 30, or more amino acids. For comparison, typically one sequence acts as a reference sequence to which one or more test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman. Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Sequence Analysis Software Package of the Genetics Computer Group (GCG), University of Wisconsin Biotechnology Center, which can be used with default parameters), or by visual inspection (see generally Ausubel et al., infra). One example of an algorithm that is suitable for determining percent sequence identity and sequence similarity is the BLAST algorithm (see, for example, Altschul et al. 1990 J. Mol. Biol. 215:403410; Gish & States. 1993 Nature Genet. 3:266-272; Madden et al. 1996 Meth. Enzymol. 266:131-141; Altschul et al. 1997 Nucleic Acids Res. 25:3389-3402; Zhang 7 Madden. 1997 Genome Res. 7:649-656). Software for performing BLAST analyses is publicly available through the National Center for Biotechnology Information. In cases where two or more polypeptide sequences differ from each other by conservative substitutions (i.e., substitutions of amino acids with chemically similar amino acids; conservative substitution tables providing functionally similar amino acids are well known in the art), the percent sequence identity or degree of homology can be adjusted upwards to correct for the conservative nature of the substitution. Means for making this adjustment are well known to those of skill in the art. See, e.g., Pearson, 1994, Methods Mol. Biol. 24:307-31 and 25:365-89.

The terms “including,” “includes,” “having,” “has,” “with,” or variants thereof as used herein are intended to be inclusive in a manner similar to the term “comprising”.

The term “milk protein” as used herein refers to a polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in a protein natively found in a mammal-produced milk (i.e., a protein that is native to a mammal-produced milk; e.g., a native whey protein or a native casein). Non-limiting examples of milk proteins include α-lactalbumin protein (e.g., amino acids 20-142 of UniProt sequence P00709, P00711, P00712, or P09462), β-lactoglobulin protein (amino acids 17-178 of UniProt sequence P02754, amino acids 19-180 of UniProt sequence P67976 or P02756), lactoferrin protein (e.g., amino acids 20 to 708 of UniProt sequence P24627. D3G9G3, or Q29477; amino acids 20 to 710 of UniProt sequence P02788), transferrin protein (e.g., amino acids 20 to 704 of UniProt sequence Q29443, or W5PF65; amino acids 20 to 698 of UniProt sequence A0A452FJF9 or P02787), serum albumin protein (e.g., amino acids 25 to 607 of UniProt sequence P02769 or P14639; amino acids 19 to 608 of UniProt sequence A0A452F7Y5; amino acids 25 to 609 of UniProt sequence P02768), lactoperoxidase protein, glycomacropeptide (GMP), β-casein protein (e.g., amino acids 16 to 224 of UniProt sequence P02666; amino acids 16 to 222 of UniProt sequence P11839 or P33048; amino acids 16 to 226 of P05814), γ-casein protein, κ-casein protein (e.g., amino acids 22 to 190 of UniProt sequence P02668; amino acids 22 to 192 of UniProt sequence P02669 or P02670; amino acids 21 to 182 of UniProt sequence P07498), α-S1-casein protein (e.g., amino acids 16 to 214 of UniProt sequence P02662, P04653, or P18626; amino acids 16 to 185 of UniProt sequence P47710), and α-S2-casein protein (e.g., amino acids 16 to 222 of UniProt sequence P02663; amino acids 16 to 223 of UniProt sequence P04654 or P33049, respectively). Non-limiting examples of polynucleotide and polypeptide sequences encoding milk proteins are disclosed in PCT filing PCT/US2015/046428 filed Aug. 21, 2015, and PCT filing PCT/US2017/48730 filed Aug. 25, 2017, which are hereby incorporated herein, in their entireties. The milk protein can be derived from any mammalian species, including but not limited to cow, human, sheep, wild sheep, goat, buffalo, camel, horse, donkey, lemur, panda, guinea pig, squirrel, bear, macaque, gorilla, chimpanzee, mountain goat, monkey, ape, cat, dog, wallaby, rat, mouse, elephant, opossum, rabbit, whale, baboons, gibbons, orangutan, mandrill, pig, wolf, fox, lion, tiger, echidna, and woolly mammoth.

The term “milk protein component” as used herein refers to a component that consists of a subset of whey proteins or of a mixture of a subset of whey proteins and a subset of caseins (i.e., consists of some but not all proteins present in, for example, whey protein concentrate, whey protein isolate, whey protein hydrolysate, casein isolate, casein concentrate, casein hydrolysate, milk protein isolate, milk protein concentrate, milk protein hydrolysate, micellar casein concentrate, sodium caseinate, or acid caseinate). The term implies that the milk proteins of which the milk protein component consists are the only milk proteins comprised in a composition provided herein (i.e., the composition comprises no other milk proteins other than the milk proteins of which the milk protein component consists).

The term “native” as used herein refers to what is found in nature in its unmodified state (e.g., a cell that is not genetically modified by a human, and that is maintained under conditions [e.g., level of oxygenation, pH, salt concentration, temperature, and nutrient (e.g., carbon, nitrogen, sulfur) availability] that are not defined by a human).

The term “one or more” as used herein refers to one, two, three, four, five, six, seven, eight, nine, ten, at least one, at least two, at least three, at least four, at least five, at least six, at least seven, at least eight, at least nine, at least ten, or more, or all of the elements subsequently listed.

The term “operably linked” as used herein refers to an arrangement of elements that allows them to be functionally related. For example, a promoter sequence is operably linked to a protein coding sequence if it controls the transcription of the protein coding sequence, and a secretion signal sequence is operably linked to a protein if the secretion signal sequence directs the protein through the secretion system of a cell. An “operably linked” element can be in contiguous linkage with another element, or act in trans or at a distance to another element. Non-limiting examples of functions that can be operably linked include control of transcription, control of translation, protein folding, and protein secretion.

The terms “optional” or “optionally” as used herein refer to a feature or structure being present or not, or an event or circumstance occurring or not. The description includes instances in which a feature or structure is present, instances in which a feature or structure is absent, instances in which an event or circumstance occurs, and instances in which an event or circumstance does not occur.

The term “polynucleotide” as used herein refers to a polymeric form of at least 2 (e.g., at least 5, at least 10, at least 20, at least 30, at least 40, at least 50, at least 100, at least 500, at least 1,000) nucleotides. The term includes both sense and antisense strands of DNA molecules (e.g., cDNA, genomic DNA, synthetic DNA) and RNA molecules (e.g., mRNA, synthetic RNA), as well as analogs of DNA or RNA containing non-natural nucleotide analogs, non-native internucleoside bonds, and/or chemical modifications. A polynucleotide may be modified chemically or biochemically or may contain non-natural or derivatized nucleotide bases. Such modifications include, for example, labels; methylation; substitution of one or more of the naturally occurring nucleotides with an analog; internucleotide modifications such as uncharged linkages (e.g., methyl phosphonates, phosphotriesters, phosphoramidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Examples of modified nucleotides are described in the art (see, for example, Malyshev et al. 2014. Nature 509:385; Li et al. 2014. J. Am. Chem. Soc. 136:826). Also included are synthetic molecules that mimic polynucleotides in their ability to bind to a designated sequence via hydrogen bonding or other chemical interaction. Such molecules are known in the art and include, for example, molecules in which peptide linkages substitute for phosphate linkages in the backbone of the molecule. Other modifications can include, for example, analogs in which the ribose ring contains a bridging moiety or other structure such as the modifications found in “locked” polynucleotides. A polynucleotide can be in any topological conformation. For instance, a polynucleotide can be single-stranded, double-stranded, triple-stranded, quadruplexed, partially double-stranded, branched, hairpinned, circular, or in a padlocked conformation. The term “polynucleotide sequence” as used herein refers to a sequence of nucleotides that are comprised in a polynucleotide or of which a polynucleotide consists.

The term “protease” as used herein refers to a protein that can hydrolyze (i.e., cleave) a peptide bond (e.g., members of enzyme classification groups EC 3.4).

The term “protease recognition or cleavage sequence” or “recognition or cleavage sequence for a protease” as used herein refers to an amino acid sequence in a polypeptide that is preferably recognized by a protease and in which a peptide bond is cleaved by the protease. The general nomenclature of positions in protease recognition or cleavage sequences are defined as described by Schechter & Berger (1967 Biochem Biophys Res Commun, 27(2):157-162; 1968 Biochem Biophys Res Commun, 32(5):898-902), which designate the cleavage site as being located between amino acid residues P1 and P1′, and incrementing numbering in the N-terminal direction of the cleaved peptide bond (i.e., P1, P2, P3, P4, etc.) and in the C-terminal (i.e., P1′, P2′, P3′, P4′, etc.).

The term “purifying” as used herein refers to a protein being substantially separated from chemicals and cellular components (e.g., cell walls, membrane lipids, chromosomes, other proteins). The term does not require (albeit allows) that the protein be separated from all other chemicals and cellular components.

The term “recombinant host cell” as used herein refers to a host cell that comprises a recombinant polynucleotide. Thus, for example, a recombinant host cell may produce a polynucleotide or polypeptide not found in the native (non-recombinant) form of the host cell, or a recombinant host cell may produce a polynucleotide or polypeptide at a level that is different from that in the native (non-recombinant) form of the host cell. It should be understood that such term is intended to refer not only to the particular subject cell but also to the progeny of such a cell. Because certain modifications may occur in succeeding generations due to either mutation or environmental influences, such progeny may not be identical to the subject cell, but are still included within the scope of the term “recombinant host cell” as used herein. A recombinant host cell can be an isolated cell or cell line grown in culture or can be a cell which resides in a living tissue or organism.

The term “recombinant β-lactoglobulin” as used herein refers to a recombinantly produced polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in a native β-lactoglobulin protein (e.g., Bos taurus β-lactoglobulin protein [amino acids 17 to 178 of UniProt sequence P02754; SEQ ID NO: 1 or 2], Ovis aries musimon β-lactoglobulin protein [UniProt sequence P67975; SEQ ID NO. 4], Ovis aries β-lactoglobulin protein [UniProt sequence P67976; SEQ ID NO: 5], Equus cahallus β-lactoglobulin protein [amino acids 19-180 of UniProt sequence P02758; SEQ ID NO: 6], Equus asinus β-lactoglobulin protein [UniProt sequence P13613; SEQ ID NO: 7], Equus caballus β-lactoglobulin protein [amino acids 19 to 181 of UniProt sequence P07380; SEQ ID NO: 8], Equus asinus β-lactoglobulin protein UniProt sequence P19647; SEQ ID NO: 91. Capra hircus β-lactoglobulin protein [amino acids 19-180 of UniProt sequence P02756; SEQ ID NO: 10]).

The term “recombinant polynucleotide” as used herein refers to a polynucleotide that is removed from its naturally occurring environment, or a polynucleotide that is not associated with all or a portion of a polynucleotide abutting or proximal to the polynucleotide when it is found in nature, or a polynucleotide that is operatively linked to a polynucleotide that it is not linked to in nature, or a polynucleotide that does not occur in nature, or a polynucleotide that contains a modification that is not found in that polynucleotide in nature (e.g., insertion, deletion, or point mutation introduced artificially, e.g., by human intervention), or a polynucleotide that is integrated into a chromosome at a heterologous site. The term can be used, e.g., to describe cloned DNA isolates, or a polynucleotide comprising a chemically synthesized nucleotide analog. A polynucleotide is also considered “recombinant” if it contains a genetic modification that does not naturally occur. For instance, an endogenous polynucleotide is considered a “recombinant polynucleotide” if it contains an insertion, deletion, or substitution of one or more nucleotides that is introduced artificially (e.g., by human intervention). Such modification can introduce into the polynucleotide a point mutation, substitution mutation, deletion mutation, insertion mutation, missense mutation, frameshift mutation, duplication mutation, amplification mutation, translocation mutation, or inversion mutation. The term includes a polynucleotide in a host cell's chromosome, as well as a polynucleotide that is not in a host cell's chromosome (e.g., a polynucleotide that is comprised in an episome). A recombinant polynucleotide in a host cell or organism may replicate using the in vivo cellular machinery of the host cell; however, such recombinant polynucleotide, although subsequently replicated intracellularly, is still considered recombinant for purposes of this invention.

The term “similar” is used herein refers to being within about +/−15% with regard to a specified attribute. The term includes being within +/−9%, +/−8%, +/−7%, +/−6%, +1-5%, +/−4%, +/−3%, +/−2%, or +/−1% with regard to the specified attribute.

The term “T-cell antigenic epitope” as used herein refers to an amino acid sequence that is recognized (e.g., bound) by a T-cell surface antigen (e.g., MHC class I and II proteins).

The term “two or more” as used herein refers to two, three, four, five, six, seven, eight, nine, ten, or more, or all of the elements subsequently listed.

The term “whey protein” as used herein refers to a polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in a whey protein natively found in a mammal-produced milk (i.e., a whey protein that is native to a mammal-produced milk; e.g., a native whey protein). Non-limiting examples of whey proteins include α-lactalbumin protein (e.g., amino acids 20-142 of UniProt sequence P00709, P00711, P00712, or P09462), β-lactoglobulin protein (amino acids 17-178 of UniProt sequence P02754, amino acids 19-180 of UniProt sequence P67976 or P02756), lactoferrin protein (e.g., amino acids 20 to 708 of UniProt sequence P24627, D3G9G3, or Q29477; amino acids 20 to 710 of UniProt sequence P02788), transferrin protein (e.g., amino acids 20 to 704 of UniProt sequence Q29443, or W5PF65; amino acids 20 to 698 of UniProt sequence A0A452FJF9 or P02787), serum albumin protein (e.g., amino acids 25 to 607 of UniProt sequence P02769 or P14639; amino acids 19 to 608 of UniProt sequence A0A452F7Y5; amino acids 25 to 609 of UniProt sequence P02768), lactoperoxidase protein, and glycomacropeptide (GMP; e.g., amino acids 127 to 190 of UniProt sequence P02668).

The term “vector” as used herein refers to a nuclei acid that can carry a polynucleotide sequence to be introduced into a host cell. Non-limiting examples of vectors include cloning vectors, expression vectors, shuttle vectors, plasmids, phage particles, viral vectors, cosmids, bacterial artificial chromosomes (BACs), yeast artificial chromosomes (YACs), virus particles (e.g., comprising heterologous polynucleotides), DNA constructs (e.g., produced by cloning or PCR amplification), and linear double-stranded molecules (e.g., PCR fragments). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome.

The term “yeast” as used herein refers to an organism of the order Saccharomycetales. Vegetative growth of yeast is by budding/blebbing of a unicellular thallus, and carbon catabolism may be fermentative.

Recitation of ranges of values herein are merely intended to serve as a shorthand method of referring individually to each separate value (fractional or integral) falling within the range inclusive of the recited minimum and maximum value, unless otherwise indicated herein, and each separate value is incorporated into the specification as if it were individually recited herein. Also, it should be understood that any numerical range recited herein is intended to include all sub-ranges subsumed therein. It should further be understood that all ranges and quantities described below are approximations and are not intended to limit the invention. Where ranges and numbers are used these can be approximate to include statistical ranges or measurement errors or variation (for example, measurements could be plus or minus 10%).

### Recombinant Milk Protein Having Attenuated or Eliminated Allergenicity

In one aspect, provided herein is a recombinant milk protein that comprises a modification compared to a corresponding native milk protein, wherein the modification attenuates or essentially eliminates allergenicity of the recombinant milk protein compared to the corresponding native milk protein.

The modification that attenuates or essentially eliminates allergenicity of the recombinant milk protein can be a modification that: introduces a protease recognition or cleavage sequence into the recombinant milk protein that is not comprised in the corresponding native milk protein (i.e., is a non-native protease recognition or cleavage sequence); and/or eliminates an allergenic epitope from the recombinant milk protein that is comprised in the corresponding native milk protein.

The modification can be a single modification, or two or more modifications. Non-limited examples of suitable modifications include: one or more amino acid substitutions, one or more amino acid deletions, one or more amino acid additions, and combinations thereof.

The recombinant milk protein according to any of the above can retain one or more functional attributes of the corresponding native milk protein.

### Modification Introducing Non-Native Protease Recognition and Cleavage Sequence

The modification that attenuates or essentially eliminates allergenicity of the recombinant milk protein according to any of the above can be a modification that introduces a non-native protease recognition or cleavage sequence in or in the vicinity (e.g., within 1, 2, 3, 4, 5, 6, 7, 8, 9, 10, 11, 12, 13, 14, or 15 amino acids) of: a solvent-exposed region of the corresponding native milk protein (i.e., a region that is on the surface of a three-dimensional structure of the corresponding native milk protein), a lipid-binding region of the corresponding native milk protein (i.e., a region in a three-dimensional structure of the corresponding native milk protein that can bind a lipid); and/or an allergenic epitope comprised in the corresponding native milk protein.

The non-native protease recognition or cleavage sequence can be a single non-native protease recognition or cleavage sequence or two or more non-native protease recognition or cleavage sequences.

By introducing a non-native protease recognition or cleavage sequence into the recombinant milk protein, a protease comprised in the gastrointestinal tract of a mammal can cleave the recombinant milk protein and destroy sequence or conformational structure of an epitope comprised in the recombinant milk protein, and thereby attenuate or essentially eliminate allergenicity of the recombinant milk protein.

The protease comprised in the gastrointestinal tract of the mammal can be produced by the mammal. Non-limiting examples of proteases comprised in the gastrointestinal tracts of mammals and produced by mammals (e.g., humans) include trypsin (e.g., cationic trypsinogen, anionic trypsinogen, mesotrypsin, pancreasin), chymotrypsin (e.g., chymotrypsinogen B1, chymotrypsinogen B2, caldecrin), elastase (e.g., elastase 2A, elastase 2B, elastase 3A, elastase 3B), carboxypeptidase A (e.g., carboxypeptidase A1, carboxypeptidase A2), carboxypeptidase B (e.g., carboxypeptidase B1, carboxypeptidase B2), and pepsin.

Alternatively, the protease comprised in the gastrointestinal tract of the mammal can be produced by a microorganism comprised in the gastrointestinal tract (e.g., comprised in the biotome) of the mammal. Non-limiting examples of proteases produced by microorganisms comprised in gastrointestinal tracts of mammals (e.g., humans) include secreted and cell wall-bound extracellular proteases produced by Alistipes putredinis, Anaerotruncus colihominis, Bacillus subtilis, Bacteroides acidofacients, Bacteroides caccae, Bacteroides capillosus, Bacteroides dorei, Bacteroides eggerthii, Bacteroides finegoldii, Bacteroides fragilis, Bacteroides intestinalis, Bacteroides ovatus, Bacteroides pectinophilus, Bacteroides sp. Bacteroides stercosis, Bacteroides thetaiotaomicron, Bacteroides uniformis, Bacteroides vulgatus, Bacteroides xylanisolvens, Bifidobacterium lactis, Blautia hansenii, Butyrivibrio crossotus, Bacillus subtilis (e.g., serine peptidases subtilisin, BPN, subtilisin E, subtilisin DY, subtilisin amylosacchariticus, subtilisin NAT, and subtilisin S41), Candida albicans (e.g., secreted aspartate proteinases SAP1, SAP2, SAP3, SAP6, SAP9, and SAP10), Clostridium asparagiforme, Clostridium leptum, Clostridium nexile, Clostridium perfringens (e.g., collagenase/ColA), Clostridium scindens, Clostridium sp. Collinsella aerofaciens, Coprococcus comes, Coprococcus eutactus, Dermatophagoides pteronyssinus, Dorea formicigenerans, Dorea longicatena, Desulfovibrio desulfuricans, Desulfovibrio fairfieldensis, Desulfovibrio piger, Entamoeba histolytica, Enterococcus faecalis (e.g., metallopeptidase gelatinase/gelE), Eubacterium ventriosum, Eubacterium hallii, Eubacterium rectale, Eubacterium siraeum, Faecalibacterium prausnitzii, Helicobacter pylori (e.g., HtrA), Holdemania filiformis, Lactobacillus acidophilus, Lactobacillus brevis, Lactobacillus bulgaricus, Lactobacillus casei, Lactobacillus paracasei, Lactobacillus plantarum, Oscillospira guillermondii, Parabacteroides distasonis, Parabacteroides johnsonii, Parabacteroides merdae, Porphyromonas gingivalis (e.g., cysteine peptidases gingipain RgpA, gingipain RgpB, and gingipain Kgp), Roseburia intestinalis, Ruminococcus bromii, Ruminococcus gnavus, Ruminococcus lactaris, Ruminococcus obeum, Ruminococcus sp. Ruminococcus torques, Staphylococcus aureus (e.g., cysteine peptidases staphopain A, staphopain B, and staphopain C), Staphylococcus epidermidis (e.g., metallopeptidase elastase/SepA), Streptococcus thermophilus, Subdoligranulum variabile, Sutterella faecalis, Sutterella parvirubra, Sutterella stercoricanis, and Sutterella wadsworthensis (see, for example, Qin et al, 2010, Nature, 464(7285): 59-65).

The modification that attenuates or essentially eliminates allergenicity of the recombinant milk protein according to any of the above can be a modification that provides for a lysine or arginine amino acid residue at P1 position of a non-native protease recognition or cleavage sequence such that the non-native protease recognition or cleavage sequence is recognized by trypsin. The modification that attenuates or essentially eliminates allergenicity of the recombinant milk protein according to any of the above can be a modification that provides for a leucine, proline, tryptophane, tyrosine, or phenylalanine amino acid residue at P1 position of a non-native protease recognition or cleavage sequence such that the non-native protease recognition or cleavage sequence is recognized by pepsin. The modification that attenuates or essentially eliminates allergenicity of the recombinant milk protein according to any of the above can be a modification that provides for a tryptophane, tyrosine, or phenylalanine amino acid residue at P1 position of a non-native protease recognition or cleavage sequence such that the non-native protease recognition or cleavage sequence is recognized by chymotrypsin. Further preferred protease recognition or cleavage sequences for proteases comprised in the gastrointestinal tract of mammals produced by mammals or by microorganisms can be deduced from data comprised in peptidase database MEROPS (https://wwvw.ebi.ac.uk/merocs/). The modification typically has minimal impact on protein structure of the recombinant milk protein compared to that of the corresponding native milk protein. Such minimal impact can be achieved by conservative amino acid substitutions (i.e., substitutions of amino acids having similar biochemical properties), and/or amino acid deletions, substitutions, and/or additions that do not create steric hindrances between side chains of amino acids in a three-dimensional conformation of the recombinant milk protein (as determined, for example, by examination using PyMol [Schrödinger, New York, N.Y.] and multi-sequence alignments [e.g., of orthologs of native milk proteins; for example, using MUSCLE (Edgar, 2004, Nucleic Acids Res, 32: 1792-1797)]).

### Modification Removing Allergenic Epitope

The modification that attenuates or essentially eliminates allergenicity of the recombinant milk protein according to any of the above can be a modification that eliminates an allergenic epitope.

The allergenic epitope can be a T-cell antigenic epitope or a B-cell antigenic epitope. Non-limiting examples of T-cell antigenic epitopes and B-cell antigenic epitopes include MHC class I (e.g., HLA-A, HLA-B, HLA-C, HLA-A1, HLA-A2, HLA-A3, HLA-A11, and subtypes thereof) binding sites, MHC class II (e.g., HLA-DPA, HLA-DPB, HLA-DPI, HLA-DP4, HLA-DP5, HLA-DP9, HLA-DQ, HLA-DR, HLA-DRB1, HLA-DRB3, HLA-DRB4, HLA-DRB5, and subtypes thereof) binding sites, non-classical (e.g., HLA-E, HLA-G, BTN3A1, CD1a, CD1b, CD1c, CD1d, MR1, and subtypes thereof) binding sites, and IgE binding sites. An allergenic epitope can be removed, for example, by changing (i.e., substituting, deleting, and/or adding) one or more (e.g., 1, 2, 3, 4, 5, 6, 7, 8, 9, 10, 11, 12, 13, 14, 15, or more) amino acids in or in the vicinity (e.g., within 1, 2, 3, 4, 5, 6, 7, 8, 9, 10, 11, 12, 13, 14, or 15 amino acids) of the allergenic epitope such that the site no longer elicits an allergenic response. An allergenic epitope can also be replaced with a polypeptide that does not comprise an allergenic epitope and that does not disrupt the structure of the recombinant milk protein (e.g., a polypeptide that is obtained from a structurally similar region of another protein [e.g., another milk protein] or from a structurally similar region of an ortholog milk protein). An allergenic epitope can be removed or modified via random or site-directed mutagenesis of a polynucleotide encoding the milk protein followed by screening for attenuated or essentially eliminated allergenicity. As a result of such modification, a contiguous amino acid segment of the recombinant milk protein having attenuated or essentially eliminated allergenicity according to any of the above can have a lesser identity to known allergenic epitopes than a corresponding contiguous amino acid segment of a corresponding native milk protein.

### Recombinant β-Lactoglobulin Protein

The recombinant milk protein having attenuated or essentially eliminated allergenicity according to any of the above can be a recombinant β-lactoglobulin protein.

The modification that attenuates or essentially eliminates allergenicity of the recombinant β-lactoglobulin protein according to any of the above can be a modification that introduces a non-native protease recognition or cleavage sequence in or in the vicinity (e.g., within 1, 2, 3, 4, 5, 6, 7, 8, 9, 10, 11, 12, 13, 14, or 15 amino acids) of a solvent-exposed region of a corresponding native β-lactoglobulin protein. Non-limiting examples of solvent-exposed regions include regions spanning from amino acid 1 to amino acid 14, amino acid 16 to amino acid 20, amino acid 27 to amino acid 31, amino acid 33 to amino acid 36, amino acid 40 to amino acid 41, amino acid 44 to amino acid 72, amino acid 74 to amino acid 79, amino acid 83 to amino acid 103, amino acid 105 to amino acid 117, amino acid 124 to amino acid 139, amino acid 141 to amino acid 146, amino acid 148 to amino acid 155, and amino acid 157 to amino acid 160 of native Bos taurus β-lactoglobulin protein (SEQ ID NO: 1 or 2), native Capra hircus β-lactoglobulin protein (SEQ ID NO: 10), Ovis aries musimon β-lactoglobulin protein (SEQ ID NO: 4), and Ovis aries β-lactoglobulin protein (SEQ ID NO: 5), and corresponding regions in homologs and orthologs.

The modification that attenuates or essentially eliminates allergenicity of the recombinant β-lactoglobulin protein according to any of the above can be a modification that introduces a non-native protease recognition or cleavage sequence in or in the vicinity (e.g., within 1, 2, 3, 4, 5, 6, 7, 8, 9, 10, 11, 12, 13, 14, or 15 amino acids) of a lipid-binding region of a corresponding native β-lactoglobulin protein. Non-limiting examples of lipid-binding regions include regions spanning from amino acid 136 to amino acid 149 of native Bos taurus β-lactoglobulin protein (SEQ ID NO: 1 or 2), native Capra hircus β-lactoglobulin protein (SEQ ID NO: 10), Ovis aries musimon β-lactoglobulin protein (SEQ ID NO: 4), and Ovis aries β-lactoglobulin protein (SEQ ID NO: 5), and corresponding regions in homologs and orthologs.

The modification that attenuates or essentially eliminates allergenicity of the recombinant β-lactoglobulin protein according to any of the above can be a modification that introduces a non-native protease recognition or cleavage sequence in or in the vicinity (e.g., within 1, 2, 3, 4, 5, 6, 7, 8, 9, 10, 11, 12, 13, 14, or 15 amino acids) of an allergenic epitope comprised in a corresponding native β-lactoglobulin protein. Non-limiting examples of allergenic epitopes (e.g., T-cell antigenic epitopes, B-cell antigenic epitopes) include regions spanning from amino acid 1 to amino acid 8, amino acid 2 to amino acid 18, amino acid 9 to amino acid 14, amino acid 25 to amino acid 40, amino acid 35 to amino acid 48, amino acid 41 to amino acid 60, amino acid 43 to amino acid 68, amino acid 47 to amino acid 62, amino acid 67 to amino acid 78, amino acid 75 to amino acid 86, amino acid 78 to amino acid 83, amino acid 84 to amino acid 91, amino acid 86 to amino acid 100, amino acid 92 to amino acid 100, amino acid 97 to amino acid 117, amino acid 102 to amino acid 124, amino acid 122 to amino acid 146, amino acid 125 to amino acid 135, amino acid 127 to amino acid 144, amino acid 135 to amino acid 147, amino acid 141 to amino acid 152, and amino acid 149 to amino acid 162 of native Bos taurus β-lactoglobulin protein (SEQ ID NO: 1 or 2), and corresponding regions in homologs and orthologs of native Bos taurus β-lactoglobulin protein.

The modification that attenuates or essentially eliminates allergenicity of the recombinant β-lactoglobulin protein according to any of the above can be a single amino acid substitution that creates one or more non-native protease recognition or cleavage sequences.

Such single amino acid substitution can be selected from the group consisting of: I12L (e.g., to produce a non-native pepsin recognition or cleavage sequence), T18K (e.g., to produce a non-native trypsin recognition or cleavage sequence), T18R (e.g., to produce a non-native trypsin recognition or cleavage sequence), I29L (e.g., to produce a non-native pepsin recognition or cleavage sequence), S30K (e.g., to produce a non-native trypsin recognition or cleavage sequence), S36Y (e.g., to produce a non-native pepsin and chymotrypsin recognition or cleavage sequence), S36L (e.g., to produce a non-native pepsin recognition or cleavage sequence), S36K (e.g., to produce a non-native trypsin recognition or cleavage sequence), K47L (e.g., to produce a non-native pepsin recognition or cleavage sequence), K47P (e.g., to produce a non-native pepsin recognition or cleavage sequence), T49K (e.g., to produce a non-native trypsin recognition or cleavage sequence), Q59R (e.g., to produce a non-native trypsin recognition or cleavage sequence), I72L (e.g., to produce a non-native pepsin recognition or cleavage sequence), I72F (e.g., to produce a non-native pepsin and chymotrypsin recognition or cleavage sequence), I72W (e.g., to produce a non-native pepsin and chymotrypsin recognition or cleavage sequence), I72Y (e.g., to produce a non-native pepsin and chymotrypsin recognition or cleavage sequence), I78L (e.g., to produce a non-native pepsin recognition or cleavage sequence), I78Y (e.g., to produce a non-native pepsin and chymotrypsin recognition or cleavage sequence), I78W (e.g., to produce a non-native pepsin and chymotrypsin recognition or cleavage sequence), I78F (e.g., to produce a non-native pepsin and chymotrypsin recognition or cleavage sequence), A86Y (e.g., to produce a non-native pepsin and chymotrypsin recognition or cleavage sequence), N90R (e.g., to produce a non-native trypsin recognition or cleavage sequence), Y102F (e.g., to produce a more accessible pepsin and chymotrypsin recognition or cleavage sequence), S110P (e.g., to produce a non-native pepsin recognition or cleavage sequence), S110L (e.g., to produce a non-native pepsin recognition or cleavage sequence), S110Y (e.g., to produce a non-native pepsin and chymotrypsin recognition or cleavage sequence), S110W (e.g., to produce a non-native pepsin and chymotrypsin recognition or cleavage sequence), S110F (e.g., to produce a non-native pepsin and chymotrypsin recognition or cleavage sequence), S110K (e.g., to produce a non-native trypsin recognition or cleavage sequence), A111P (e.g., to produce a non-native pepsin recognition or cleavage sequence), A111K (e.g., to produce a non-native trypsin recognition or cleavage sequence), A111L (e.g., to produce a non-native pepsin recognition or cleavage sequence), A111Y (e.g., to produce a non-native pepsin and chymotrypsin recognition or cleavage sequence), A111W (e.g., to produce a non-native pepsin and chymotrypsin recognition or cleavage sequence), A111F (e.g., to produce a non-native pepsin and chymotrypsin recognition or cleavage sequence), T125K (e.g., to produce a non-native trypsin recognition or cleavage sequence), T125F (e.g., to produce a non-native pepsin and chymotrypsin recognition or cleavage sequence), T125W (e.g., to produce a non-native pepsin and chymotrypsin recognition or cleavage sequence), T125Y (e.g., to produce a non-native pepsin and chymotrypsin recognition or cleavage sequence), E127K (e.g., to produce a non-native trypsin recognition or cleavage sequence), E127L (e.g., to produce a non-native pepsin recognition or cleavage sequence), E127W (e.g., to produce a non-native pepsin and chymotrypsin recognition or cleavage sequence), E127F (e.g., to produce a non-native pepsin and chymotrypsin recognition or cleavage sequence), E127Y (e.g., to produce a non-native pepsin and chymotrypsin recognition or cleavage sequence), V128L (e.g., to produce a non-native pepsin recognition or cleavage sequence), D137R (e.g., to produce a non-native trypsin recognition or cleavage sequence), A142P (e.g., to produce a non-native pepsin recognition or cleavage sequence), A142L (e.g., to produce a non-native pepsin recognition or cleavage sequence), H146R (e.g., to produce a non-native trypsin recognition or cleavage sequence), T154K (e.g., to produce a non-native trypsin recognition or cleavage sequence), T154Y (e.g., to produce a non-native pepsin and chymotrypsin recognition or cleavage sequence), T154L (e.g., to produce a non-native pepsin recognition or cleavage sequence), T154W (e.g., to produce a non-native pepsin and chymotrypsin recognition or cleavage sequence), T154F (e.g., to produce a non-native pepsin and chymotrypsin recognition or cleavage sequence), and Q159R (e.g., to produce a non-native trypsin recognition or cleavage sequence) of native Bos taurus β-lactoglobulin protein (SEQ ID NO: 1 or 2), and corresponding single amino acid substitutions in homologs and orthologs of native Bos taurus β-lactoglobulin protein.

Such single amino acid substitution can be selected from the group consisting of: I12L (e.g., to produce a non-native pepsin recognition or cleavage sequence), T18K (e.g., to produce anon-native trypsin recognition or cleavage sequence), T18R (e.g., to produce anon-native trypsin recognition or cleavage sequence), I29L (e.g., to produce a non-native pepsin recognition or cleavage sequence), S30K (e.g., to produce a non-native trypsin recognition or cleavage sequence), S36Y (e.g., to produce a non-native pepsin and chymotrypsin recognition or cleavage sequence), S36L (e.g., to produce a non-native pepsin recognition or cleavage sequence), S36K (e.g., to produce a non-native trypsin recognition or cleavage sequence), K47L (e.g., to produce a non-native pepsin recognition or cleavage sequence), K47P (e.g., to produce a non-native pepsin recognition or cleavage sequence), T49K (e.g., to produce a non-native trypsin recognition or cleavage sequence), Q59R (e.g., to produce a non-native trypsin recognition or cleavage sequence), I72L (e.g., to produce a non-native pepsin recognition or cleavage sequence), I72F (e.g., to produce a non-native pepsin and chymotrypsin recognition or cleavage sequence), I72W (e.g., to produce a non-native pepsin and chymotrypsin recognition or cleavage sequence), I72Y (e.g., to produce a non-native pepsin and chymotrypsin recognition or cleavage sequence), I78L (e.g., to produce a non-native pepsin recognition or cleavage sequence), I78Y (e.g., to produce a non-native pepsin and chymotrypsin recognition or cleavage sequence), I78W (e.g., to produce a non-native pepsin and chymotrypsin recognition or cleavage sequence), I78F (e.g., to produce a non-native pepsin and chymotrypsin recognition or cleavage sequence), A86Y (e.g., to produce a non-native pepsin and chymotrypsin recognition or cleavage sequence), N90R (e.g., to produce a non-native trypsin recognition or cleavage sequence), Y102F (e.g., to produce a more accessible pepsin and chymotrypsin recognition or cleavage sequence), S110P (e.g., to produce a non-native pepsin recognition or cleavage sequence), S110L (e.g., to produce a non-native pepsin recognition or cleavage sequence), S110Y (e.g., to produce a non-native pepsin and chymotrypsin recognition or cleavage sequence), S110W (e.g., to produce a non-native pepsin and chymotrypsin recognition or cleavage sequence), S110F (e.g., to produce a non-native pepsin and chymotrypsin recognition or cleavage sequence), S110K (e.g., to produce a non-native trypsin recognition or cleavage sequence), A111P (e.g., to produce a non-native pepsin recognition or cleavage sequence), A111K (e.g., to produce a non-native trypsin recognition or cleavage sequence), A111L (e.g., to produce a non-native pepsin recognition or cleavage sequence), A111Y (e.g., to produce a non-native pepsin and chymotrypsin recognition or cleavage sequence), A111W (e.g., to produce a non-native pepsin and chymotrypsin recognition or cleavage sequence), A111F (e.g., to produce a non-native pepsin and chymotrypsin recognition or cleavage sequence), T125K (e.g., to produce a non-native trypsin recognition or cleavage sequence), T125F (e.g., to produce a non-native pepsin and chymotrypsin recognition or cleavage sequence), T125W (e.g., to produce a non-native pepsin and chymotrypsin recognition or cleavage sequence), T125Y (e.g., to produce a non-native pepsin and chymotrypsin recognition or cleavage sequence), E127K (e.g., to produce a non-native trypsin recognition or cleavage sequence), E127L (e.g., to produce a non-native pepsin recognition or cleavage sequence), E127W (e.g., to produce a non-native pepsin and chymotrypsin recognition or cleavage sequence), E127F (e.g., to produce a non-native pepsin and chymotrypsin recognition or cleavage sequence), E127Y (e.g., to produce a non-native pepsin and chymotrypsin recognition or cleavage sequence), V128L (e.g., to produce a non-native pepsin recognition or cleavage sequence), D137R (e.g., to produce a non-native trypsin recognition or cleavage sequence), A142P (e.g., to produce a non-native pepsin recognition or cleavage sequence), A142L (e.g., to produce a non-native pepsin recognition or cleavage sequence), H146R (e.g., to produce a non-native trypsin recognition or cleavage sequence), T154K (e.g., to produce a non-native trypsin recognition or cleavage sequence), T154Y (e.g., to produce a non-native pepsin and chymotrypsin recognition or cleavage sequence), T154L (e.g., to produce a non-native pepsin recognition or cleavage sequence), T154W (e.g., to produce a non-native pepsin and chymotrypsin recognition or cleavage sequence), T154F (e.g., to produce a non-native pepsin and chymotrypsin recognition or cleavage sequence), and Q159R (e.g., to produce a non-native trypsin recognition or cleavage sequence) of native Ovis aries musimon, Ovis aries, or Capra hircus β-lactoglobulin protein (SEQ ID NO: 4, 5, or 10, respectively), and corresponding single amino acid substitutions in homologs and orthologs of native Ovis aries musimon, Ovis aries, or Capra hircus β-lactoglobulin protein.

Such single amino acid substitution can be selected from the group consisting of: I29L (e.g., to produce a non-native pepsin recognition or cleavage sequence), S30K (e.g., to produce a non-native trypsin recognition or cleavage sequence), S36Y (e.g., to produce a non-native pepsin and chymotrypsin recognition or cleavage sequence), S36L (e.g., to produce a non-native pepsin recognition or cleavage sequence), S36K (e.g., to produce a non-native trypsin recognition or cleavage sequence), R47L (e.g., to produce a non-native pepsin recognition or cleavage sequence), R47P (e.g., to produce a non-native pepsin recognition or cleavage sequence), T49K (e.g., to produce a non-native trypsin recognition or cleavage sequence), F72L (e.g., to produce a non-native pepsin recognition or cleavage sequence), F72W (e.g., to produce a non-native pepsin and chymotrypsin recognition or cleavage sequence), F72Y (e.g., to produce a non-native pepsin and chymotrypsin recognition or cleavage sequence), S78L (e.g., to produce a non-native pepsin recognition or cleavage sequence), S78Y (e.g., to produce a non-native pepsin and chymotrypsin recognition or cleavage sequence), S78W (e.g., to produce a non-native pepsin and chymotrypsin recognition or cleavage sequence), S78F (e.g., to produce a non-native pepsin and chymotrypsin recognition or cleavage sequence), D90R (e.g., to produce a non-native trypsin recognition or cleavage sequence), Y102F (e.g., to produce a more accessible pepsin and chymotrypsin recognition or cleavage sequence), A111P (e.g., to produce anon-native pepsin recognition or cleavage sequence), A111K (e.g., to produce a non-native trypsin recognition or cleavage sequence), A111L (e.g., to produce a non-native pepsin recognition or cleavage sequence), A111Y (e.g., to produce a non-native pepsin and chymotrypsin recognition or cleavage sequence), A111W (e.g., to produce a non-native pepsin and chymotrypsin recognition or cleavage sequence), A111F (e.g., to produce a non-native pepsin and chymotrypsin recognition or cleavage sequence), T125K (e.g., to produce anon-native trypsin recognition or cleavage sequence), T125F (e.g., to produce a non-native pepsin and chymotrypsin recognition or cleavage sequence), T125W (e.g., to produce a non-native pepsin and chymotrypsin recognition or cleavage sequence), T125Y (e.g., to produce a non-native pepsin and chymotrypsin recognition or cleavage sequence), V128L (e.g., to produce a non-native pepsin recognition or cleavage sequence), T154K (e.g., to produce a non-native trypsin recognition or cleavage sequence), T154Y (e.g., to produce a non-native pepsin and chymotrypsin recognition or cleavage sequence), T154L (e.g., to produce a non-native pepsin recognition or cleavage sequence), T154W (e.g., to produce a non-native pepsin and chymotrypsin recognition or cleavage sequence), and T154F (e.g., to produce a non-native pepsin and chymotrypsin recognition or cleavage sequence) of native Equus caballus β-lactoglobulin protein (SEQ ID NO: 6), and corresponding single amino acid substitutions in homologs and orthologs of native Equus caballus β-lactoglobulin protein.

Such single amino acid substitution can be selected from the group consisting of: I29L (e.g., to produce a non-native pepsin recognition or cleavage sequence), S30K (e.g., to produce a non-native trypsin recognition or cleavage sequence), R47L (e.g., to produce a non-native pepsin recognition or cleavage sequence), R47P (e.g., to produce a non-native pepsin recognition or cleavage sequence), T49K (e.g., to produce a non-native trypsin recognition or cleavage sequence), F72L (e.g., to produce a non-native pepsin recognition or cleavage sequence), F72W (e.g., to produce a non-native pepsin and chymotrypsin recognition or cleavage sequence), F72Y (e.g., to produce anon-native pepsin and chymotrypsin recognition or cleavage sequence), S78L (e.g., to produce a non-native pepsin recognition or cleavage sequence), S78Y (e.g., to produce a non-native pepsin and chymotrypsin recognition or cleavage sequence), S78W (e.g., to produce a non-native pepsin and chymotrypsin recognition or cleavage sequence), S78F (e.g., to produce a non-native pepsin and chymotrypsin recognition or cleavage sequence), D90R (e.g., to produce a non-native trypsin recognition or cleavage sequence), Y102F (e.g., to produce a more accessible pepsin and chymotrypsin recognition or cleavage sequence), A111P (e.g., to produce a non-native pepsin recognition or cleavage sequence), A111K (e.g., to produce a non-native trypsin recognition or cleavage sequence), A111L (e.g., to produce a non-native pepsin recognition or cleavage sequence), A111Y (e.g., to produce a non-native pepsin and chymotrypsin recognition or cleavage sequence), A111W (e.g., to produce a non-native pepsin and chymotrypsin recognition or cleavage sequence), A111F (e.g., to produce a non-native pepsin and chymotrypsin recognition or cleavage sequence), T125K (e.g., to produce anon-native trypsin recognition or cleavage sequence), T125F (e.g., to produce a non-native pepsin and chymotrypsin recognition or cleavage sequence), T125W (e.g., to produce a non-native pepsin and chymotrypsin recognition or cleavage sequence), T125Y (e.g., to produce a non-native pepsin and chymotrypsin recognition or cleavage sequence), V128L (e.g., to produce a non-native pepsin recognition or cleavage sequence), T154K (e.g., to produce a non-native trypsin recognition or cleavage sequence), T154Y (e.g., to produce a non-native pepsin and chymotrypsin recognition or cleavage sequence), T154L (e.g., to produce a non-native pepsin recognition or cleavage sequence), T154W (e.g., to produce a non-native pepsin and chymotrypsin recognition or cleavage sequence), and T154F (e.g., to produce a non-native pepsin and chymotrypsin recognition or cleavage sequence) of native Equus asinus β-lactoglobulin protein (SEQ ID NO: 7), and corresponding single amino acid substitutions in homologs and orthologs of native Equus asinus β-lactoglobulin protein.

Such single amino acid substitution can be selected from the group consisting of: I29L (e.g., to produce a non-native pepsin recognition or cleavage sequence), S30K (e.g., to produce a non-native trypsin recognition or cleavage sequence), S36Y (e.g., to produce a non-native pepsin and chymotrypsin recognition or cleavage sequence), S36L (e.g., to produce a non-native pepsin recognition or cleavage sequence), S36K (e.g., to produce a non-native trypsin recognition or cleavage sequence), R47L (e.g., to produce a non-native pepsin recognition or cleavage sequence), R47P (e.g., to produce a non-native pepsin recognition or cleavage sequence), T49K (e.g., to produce a non-native trypsin recognition or cleavage sequence), V72L (e.g., to produce a non-native pepsin recognition or cleavage sequence), V72F (e.g., to produce a non-native pepsin and chymotrypsin recognition or cleavage sequence), V72W (e.g., to produce a non-native pepsin and chymotrypsin recognition or cleavage sequence), V72Y (e.g., to produce a non-native pepsin and chymotrypsin recognition or cleavage sequence), D78L (e.g., to produce a non-native pepsin recognition or cleavage sequence), D78Y (e.g., to produce a non-native pepsin and chymotrypsin recognition or cleavage sequence), D78W (e.g., to produce a non-native pepsin and chymotrypsin recognition or cleavage sequence), D78F (e.g., to produce a non-native pepsin and chymotrypsin recognition or cleavage sequence), Y102F (e.g., to produce a more accessible pepsin and chymotrypsin recognition or cleavage sequence), T126K (e.g., to produce a non-native trypsin recognition or cleavage sequence), T126F (e.g., to produce a non-native pepsin and chymotrypsin recognition or cleavage sequence), T126W (e.g., to produce a non-native pepsin and chymotrypsin recognition or cleavage sequence), T126Y (e.g., to produce a non-native pepsin and chymotrypsin recognition or cleavage sequence), V129L (e.g., to produce a non-native pepsin recognition or cleavage sequence), and S138R (e.g., to produce a non-native trypsin recognition or cleavage sequence) of native Equus caballus or Equus asinus β-lactoglobulin protein (SEQ ID NO: 8 or 9, respectively), and corresponding single amino acid substitutions in homologs and orthologs of native Equus caballus or Equus asinus β-lactoglobulin protein.

Such single amino acid substitution can be selected from the group consisting of H147R (e.g., to produce a non-native trypsin recognition or cleavage sequence) of native Equus asinus β-lactoglobulin protein (SEQ ID NO: 9), and corresponding single amino acid substitutions in homologs and orthologs of native Equus asinus β-lactoglobulin protein.

Alternatively, the modification that attenuates or essentially eliminates allergenicity of the recombinant β-lactoglobulin protein according to any of the above can consist of two or more amino acid substitutions that create one or more non-native protease recognition or cleavage sequences.

Such two or more amino acid substitutions can comprise or consist of two or more amino acid substitutions selected from the group consisting of: I12L, T18K, T18R, I29L, S30K, S36Y, S36L, S36K, K47L, K47P, T49K, Q59R, I72L, I72F, I72W, I72Y, I78L, I78Y, I78W, I78F, A86Y, N90R, Y102F, S110P, S110L, S110Y, S110W, S110F, S110K, A111P, A111K, A111L, A111Y, A111W, A111F, T125K, T125F, T125W, T125Y, E127K, E127L, E127W, E127F, E127Y, V128L, D137R, A142P, A142L, H146R, T154K, T154Y, T154L, T154W, T154F, Q159R, and combinations thereof of native Bos taurus β-lactoglobulin protein (SEQ ID NO: 1 or 2), and corresponding two or more amino acid substitutions in homologs and orthologs of native Bos taurus β-lactoglobulin protein. Such two or more amino acid substitutions can comprise or consist of I12L and one or more of: T18K or T18R, I29L, S30K, S36Y or S36L or S36K, K47L or K47P, T49K, Q59R, I72L or I72F or I72W or I72Y, I78L or I78Y or I78W or I78F, A86Y, N90R, Y102F, S110P or S110L or S110Y or S110W or S110F or S110K, A111P or A111K or A111L or A111Y, A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of T18K or T18R and one or more of: I29L, S30K, S36Y or S36L or S36K, K47L or K47P, T49K, Q59R, I72L or I72F or I72W or I72Y, I78L or I78Y or I78W or I78F, A86Y, N90R, Y102F, S110P or S110L or S110Y or S110W or S110F or S110K, A111P or A111K or A111L or A111Y, A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of I29L and one or more of: S30K, S36Y or S36L or S36K, K47L or K47P, T49K, Q59R, I72L or I72F or I72W or I72Y, I78L or I78Y or I78W or I78F, A86Y, N90R, Y102F, S110P or S110L or S110Y or S110W or S110F or S110K, A111P or A111K or A111L or A111Y, A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of S30K and one or more of: S36Y or S36L or S36K, K47L or K47P, T49K, Q59R, I72L or I72F or I72W or I72Y, I78L or I78Y or I78W or I78F, A86Y, N90R, Y102F, S110P or S110L or S110Y or S110W or S110F or S110K, A111P or A111K or A111L or A111Y, A111W or A111F, T125K or T125F or T125W or T125Y, E127K or E127L or E127W or E127F or E127Y, V128L, D137R. A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of S36Y or S36L or S36K and one or more of: K47L or K47P, T49K, Q59R, I72L or I72F or I72W or I72Y, I78L or I78Y or I78W or I78F, A86Y, N90R, Y102F, S110P or S110L or S110Y or S110W or S110F or S110K, A111P or A111K or A111L or A111Y, A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of K47L or K47P and one or more of: T49K, Q59R, I72L or I72F or I72W or I72Y, I78L or I78Y or I78W or I78F, A86Y, N90R, Y102F, S110P or S110L or S110Y or S110W or S110F or S110K, A111P or A111K or A111L or A111Y, A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of T49K and one or more of: Q59R, I72L or I72F or I72W or I72Y, I78L or I78Y or I78W or I78F, A86Y, N90R, Y102F, S110P or S110L or S110Y or S110W or S110F or S110K, A111P or A111K or A111L or A111Y, A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of Q59R and one or more of: I72L or I72F or I72W or I72Y, I78L or I78Y or I78W or I78F, A86Y, N90R, Y102F, S110P or S110L or S110Y or S110W or S110F or S110K, A111P or A111K or A111L or A111Y, A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of I72L or I72F or I72W or I72Y and one or more of: I78L or I78Y or I78W or I78F, A86Y, N90R, Y102F, S110P or S110L or S110Y or S110W or S110F or S110K, A111P or A111K or A111L or A111Y, A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of I78L or I78Y or I78W or I78F and one or more of: A86Y, N90R, Y102F, S110P or S110L or S110Y or S110W or S110F or S110K, A111P or A111K or A111L or A111Y, A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of A86Y and one or more of: N90R, Y102F, S110P or S110L or S110Y or S110W or S110F or S110K, A111P or A111K or A111L or A111Y, A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of N90R and one or more of; Y102F, S110P or S110L or S110Y or S110W or S110F or S110K, A111P or A111K or A111L or A111Y, A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of Y102F and one or more of: S110P or S110L or S110Y or S110W or S110F or S110K, A111P or A111K or A111L or A111Y, A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of S110P or S110L or S110Y or S110W or S110F or S110K and one or more of: A111P or A111K or A111L or A111Y, A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of A111P or A111K or A111L or A111Y, A111W or A111F and one or more of: T125K or T125F or T125W or T125Y, E127K or E127L or E127W or E127F or E127Y, V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of T125K or T125F or T125W or T125Y and one or more of: E127K or E127L or E127W or E127F or E127Y, V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of E127K or E127L or E127W or E127F or E127Y and one or more of: V128L, D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of V128L and one or more of: D137R, A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of D137R and one or more of: A142P or A142L, H146R, T154K or T154Y or T154L or T154W or T154F, and Q159R of native Bos taurus β-lactoglobulin protein (SEQ ID NO: 1 or 2). Such two or more amino acid substitutions can comprise or consist of A142P or A142L and one or more of: H146R, T154K or T154Y or T154L or T154W or T154F, and Q159R of native Bos taurus β-lactoglobulin protein (SEQ ID NO: 1 or 2). Such two or more amino acid substitutions can comprise or consist of H146R and one or more of: T154K or T154Y or T154L or T154W or T154F, and Q159R of native Bos taurus β-lactoglobulin protein (SEQ ID NO: 1 or 2). Such two or more amino acid substitutions can comprise or consist of T154K or T154Y or T154L or T154W or T154F and Q159R of native Bos taurus β-lactoglobulin protein (SEQ ID NO: 1 or 2).

Such two or more amino acid substitutions can comprise or consist of two or more amino acid substitutions selected from the group consisting of: I12L, T18K, T18R, I29L, S30K, S36Y, S36L, S36K, K47L, K47P, T49K, Q59R, I72L, I72F, I72W, I72Y, I78L, I78Y, I78W, I78F, A86Y, N90R, Y102F, S110P, S110L, S110Y, S110W, S110F, S110K, A111P, A111K, A111L, A111Y, A111W, A111F, T125K, T125F, T125W, T125Y, E127K, E127L, E127W, E127F, E127Y, V128L, D137R, A142P, A142L, H146R, T154K, T154Y, T154L, T154W, T154F, Q159R, and combinations thereof of native Ovis aries musimon, Ovis aries, or Capra hircus β-lactoglobulin protein (SEQ ID NO: 4, 5, or 10, respectively), and corresponding two or more amino acid substitutions in homologs and orthologs of native Ovis aries musimon, Ovis aries, or Capra hircus β-lactoglobulin protein. Such two or more amino acid substitutions can comprise or consist of I12L and one or more of: T18K or T18R, I29L, S30K, S36Y or S36L or S36K, K47L or K47P, T49K, Q59R, I72L or I72F or I72W or I72Y, I78L or I78Y or I78W or I78F, A86Y, N90R, Y102F, S110P or S110L or S110Y or S110W or S110F or S110K, A111P or A111K or A111L or A111Y or A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of T18K or T18R and one or more of: I29L, S30K, S36Y or S36L or S36K, K47L or K47P, T49K, Q59R, I72L or I72F or I72W or I72Y, I78L or I78Y or I78W or I78F, A86Y, N90R, Y102F, S110P or S110L or S110Y or S110W or S110F or S110K, A111P or A111K or A111L or A111Y or A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of I29L and one or more of: S30K, S36Y or S36L or S36K, K47L or K47P, T49K, Q59R, I72L or I72F or I72W or I72Y, I78L or I78Y or I78W or I78F, A86Y, N90R, Y102F, S110P or S110L or S110Y or S110W or S110F or S110K, A111P or A111K or A111L or A111Y or A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of S30K and one or more of: S36Y or S36L or S36K, K47L or K47P, T49K, Q59R, I72L or I72F or I72W or I72Y, I78L or I78Y or I78W or I78F, A86Y, N90R, Y102F, S110P or S110L or S110Y or S110W or S110F or S110K, A111P or A111K or A111L or A111Y or A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of S36Y or S36L or S36K and one or more of: K47L or K47P, T49K, Q59R, I72L or I72F or I72W or I72Y, I78L or I78Y or I78W or I78F, A86Y, N90R, Y102F, S110P or S110L or S110Y or S110W or S110F or S110K, A111P or A111K or A111L or A111Y or A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of K47L or K47P and one or more of; T49K, Q59R, I72L or I72F or I72W or I72Y, I78L or I78Y or I78W or I78F, A86Y, N90R, Y102F, S10P or S110L or S110Y or S110W or S110F or S110K, A111P or A111K or A111L or A111Y or A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of T49K and one or more of; Q59R, I72L or I72F or I72W or I72Y, I78L or I78Y or I78W or I78F, A86Y, N90R, Y102F, S110P or S110L or S110Y or S110W or S110F or S110K, A111P or A111K or A111L or A111Y or A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of Q59R and one or more of: I72L or I72F or I72W or I72Y, I78L or I78Y or I78W or I78F, A86Y, N90R, Y102F, S110P or S110L or S110Y or S110W or S110F or S110K, A111P or A111K or A111L or A111Y or A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of I72L or I72F or I72W or I72Y and one or more of; I78L or I78Y or I78W or I78F, A86Y, N90R, Y102F, S110P or S110L or S110Y or S110W or S110F or S110K, A111P or A111K or A111L or A111Y or A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of I78L or I78Y or I78W or I78F and one or more of: A86Y, N90R, Y102F, S110P or S110L or S110Y or S110W or S110F or S110K, A111P or A111K or A111L or A111Y or A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of A86Y and one or more of: N90R, Y102F, S110P or S110L or S110Y or S110W or S110F or S110K, A111P or A111K or A111L or A111Y or A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of N90R and one or more of; Y102F, S110P or S110L or S110Y or S110W or S110F or S110K, A111P or A111K or A111L or A111Y or A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of Y102F and one or more of: S110P or S110L or S110Y or S110W or S110F or S110K, A111P or A111K or A111L or A111Y or A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of S110P or S110L or S110Y or S110W or S110F or S110K and one or more of: A111P or A111K or A111L or A111Y or A111W or A111F, T125K or T125F or T125W or T125Y, E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of A111P or A111K or A111L or A111Y or A111W or A111F and one or more of: T125K or T125F or T125W or T125Y, E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of T125K or T125F or T125W or T125Y and one or more of: E127K or E127L or E127W or E127F or E127Y, V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of E127K or E127L or E127W or E127F or E127Y and one or more of: V128L, D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of V128L and one or more of: D137R, A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of D137R and one or more of: A142P or A142L, H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of A142P or A142L and one or more of; H146R, T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of H146R and one or more of: T154K or T154Y or T154L or T154W or T154F, and Q159R of native Ovis aries musimon, Ovis aries, or Capra hircus β-lactoglobulin protein (SEQ ID NO: 4, 5, or 10, respectively). Such two or more amino acid substitutions can comprise or consist of T154K or T154Y or T154L or T154W or T154F and Q159R of native Ovis aries musimon, Ovis aries, or Capra hircus β-lactoglobulin protein (SEQ ID NO: 4, 5, or 10, respectively).

Such two or more amino acid substitutions can comprise or consist of two or more amino acid substitutions selected from the group consisting of: I29L, S30K, S36Y, S36L, S36K, R47L, R47P, T49K, F72L, F72W, F72Y, S78L, S78Y, S78W, S78F, D90R, Y102F, A111P, A111K, A111L, A111Y, A111W, A111F, T125K, T125F, T125W, T125Y, V128L, T154K, T154Y, T154L, T154W, T154F, and combinations thereof of native Equus caballus β-lactoglobulin protein (SEQ ID NO: 6), and corresponding two or more amino acid substitutions in homologs and orthologs of native Equus caballus β-lactoglobulin protein. Such two or more amino acid substitutions can comprise or consist of I29L and one or more of: S30K, S36Y or S36L or S36K, R47L or R47P, T49K, F72L or F72W or F72Y, S78L or S78Y or S78W or S78F, D90R, Y102F, A111P or A111K or A111L or A111Y or A111W or A111F, T125K or T125F or T125W or T125Y, V128L, and T154K or T154Y or T154L or T154W or T154F of native Equus caballus β-lactoglobulin protein (SEQ ID NO: 6). Such two or more amino acid substitutions can comprise or consist of S30K and one or more of: S36Y or S36L or S36K, R47L or R47P, T49K, F72L or F72W or F72Y, S78L or S78Y or S78W or S78F, D90R, Y102F, A111P or A111K or A111L or A111Y or A111W or A111F, T125K or T125F or T125W or T125Y, V128L, and T154K or T154Y or T154L or T154W or T154F of native Equus caballus β-lactoglobulin protein (SEQ ID NO: 6). Such two or more amino acid substitutions can comprise or consist of S36Y or S36L or S36K and one or more of: R47L or R47P, T49K, F72L or F72W or F72Y, S78L or S78Y or S78W or S78F, D90R, Y102F, A111P or A111K or A111L or A111Y or A111W or A111F, T125K or T125F or T125W or T125Y, V128L, and T154K or T154Y or T154L or T154W or T154F of native Equus caballus β-lactoglobulin protein (SEQ ID NO: 6). Such two or more amino acid substitutions can comprise or consist of R47L or R47P and one or more of: T49K, F72L or F72W or F72Y, S78L or S78Y or S78W or S78F, D90R, Y102F, A111P or A111K or A111L or A111Y or A111W or A111F, T125K or T125F or T125W or T125Y, V128L, and T154K or T154Y or T154L or T154W or T154F of native Equus caballus β-lactoglobulin protein (SEQ ID NO: 6). Such two or more amino acid substitutions can comprise or consist of T49K and one or more of: F72L or F72W or F72Y, S78L or S78Y or S78W or S78F, D90R, Y102F, A111P or A111K or A111L or A111Y or A111W or A111F, T125K or T125F or T125W or T125Y, V128L, and T154K or T154Y or T154L or T154W or T154F of native Equus caballus β-lactoglobulin protein (SEQ ID NO: 6). Such two or more amino acid substitutions can comprise or consist of F72L or F72W or F72Y and one or more of: S78L or S78Y or S78W or S78F, D90R, Y102F, A111P or A111K or A111L or A111Y or A111W or A111F, T125K or T125F or T125W or T125Y, V128L, and T154K or T154Y or T154L or T154W or T154F of native Equus caballus β-lactoglobulin protein (SEQ ID NO: 6). Such two or more amino acid substitutions can comprise or consist of S78L or S78Y or S78W or S78F and one or more of: D90R, Y102F, A111P or A111K or A111L or A111Y or A111W or A111F, T125K or T125F or T125W or T125Y, V128L, and T154K or T154Y or T154L or T154W or T154F of native Equus caballus β-lactoglobulin protein (SEQ ID NO: 6). Such two or more amino acid substitutions can comprise or consist of D90R and one or more of: Y102F, A111P or A111K or A111L or A111Y or A111W or A111F, T125K or T125F or T125W or T125Y, V128L, and T154K or T154Y or T154L or T154W or T154F of native Equus caballusβ-lactoglobulin protein (SEQ ID NO: 6). Such two or more amino acid substitutions can comprise or consist of Y102F and one or more of: A111P or A111K or A111L or A111Y or A111W or A111F, T125K or T125F or T125W or T125Y, V128L, and T154K or T154Y or T154L or T154W or T154F of native Equus caballus β-lactoglobulin protein (SEQ ID NO: 6). Such two or more amino acid substitutions can comprise or consist of A111P or A111K or A111L or A111Y or A111W or A111F and one or more of: T125K or T125F or T125W or T125Y, V128L, and T154K or T154Y or T154L or T154W or T154F of native Equus caballus β-lactoglobulin protein (SEQ ID NO: 6). Such two or more amino acid substitutions can comprise or consist of T125K or T125F or T125W or T125Y and one or more of: V128L, and T154K or T154Y or T154L or T154W or T154F of native Equus caballus β-lactoglobulin protein (SEQ ID NO: 6). Such two or more amino acid substitutions can comprise or consist of V128L and T154K or T154Y or T154L or T154W or T154F of native Equus caballus β-lactoglobulin protein (SEQ ID NO: 6).

Such two or more amino acid substitutions can comprise or consist of two or more amino acid substitutions selected from the group consisting of: I29L, S30K, R47L, R47P, T49K, F72L, F72W, F72Y, S78L, S78Y, S78W, S78F, D90R, Y102F, A111P, A111K, A111L, A111Y, A111W, A111F, T125K, T125F, T125W, T125Y, V128L, T154K, T154Y, T154L, T154W, T154F, and combinations thereof of native Equus asinus β-lactoglobulin protein (SEQ ID NO: 7), and corresponding two or more amino acid substitutions in homologs and orthologs of native Equus asinus β-lactoglobulin protein. Such two or more amino acid substitutions can comprise or consist of I29L and one or more of: S30K, R47L or R47P, T49K, F72L or F72W or F72Y, S78L or S78Y or S78W or S78F, D90R, Y102F, A111P or A111K or A111L or A111Y or A111W or A111F, T125K or T125F or T125W or T125Y, V128L, and T154K or T154Y or T154L or T154W or T154F of native Equus asinus β-lactoglobulin protein (SEQ ID NO: 7). Such two or more amino acid substitutions can comprise or consist of S30K and one or more of: R47L or R47P, T49K, F72L or F72W or F72Y, S78L or S78Y or S78W or S78F, D90R, Y102F, A111P or A111K or A111L or A111Y or A111W or A111F, T125K or T125F or T125W or T125Y, V128L, and T154K or T154Y or T154L or T154W or T154F of native Equus asinus β-lactoglobulin protein (SEQ ID NO: 7). Such two or more amino acid substitutions can comprise or consist of R47L or R47P and one or more of: T49K, F72L or F72W or F72Y, S78L or S78Y or S78W or S78F, D90R, Y102F, A111P or A111K or A111L or A111Y or A111W or A111F, T125K or T125F or T125W or T125Y, V128L, and T154K or T154Y or T154L or T154W or T154F of native Equus asinus β-lactoglobulin protein (SEQ ID NO: 7). Such two or more amino acid substitutions can comprise or consist of T49K and one or more of: F72L or F72W or F72Y, S78L or S78Y or S78W or S78F, D90R, Y102F, A111P or A111K or A111L or A111Y or A111W or A111F, T125K or T125F or T125W or T125Y, V128L, and T154K or T154Y or T154L or T154W or T154F of native Equus asinus β-lactoglobulin protein (SEQ ID NO: 7). Such two or more amino acid substitutions can comprise or consist of F72L or F72W or F72Y and one or more of: S78L or S78Y or S78W or S78F, D90R, Y102F, A11P or A111K or A111L or A111Y or A111W or A111F, T125K or T125F or T125W or T125Y, V128L, and T154K or T154Y or T154L or T154W or T154F of native Equus asinus β-lactoglobulin protein (SEQ ID NO: 7). Such two or more amino acid substitutions can comprise or consist of S78L or S78Y or S78W or S78F and one or more of: D90R, Y102F, A111P or A111K or A111L or A111Y or A111W or A111F, T125K or T125F or T125W or T125Y, V128L, and T154K or T154Y or T154L or T154W or T154F of native Equus asinus β-lactoglobulin protein (SEQ ID NO: 7). Such two or more amino acid substitutions can comprise or consist of D90R and one or more of: Y102F, A111P or A111K or A111L or A111Y or A111W or A111F, T125K or T125F or T125W or T125Y, V128L, and T154K or T154Y or T154L or T154W or T154F of native Equus asinus β-lactoglobulin protein (SEQ ID NO: 7). Such two or more amino acid substitutions can comprise or consist of Y102F and one or more of: A111P or A111K or A111L or A111Y or A111W or A111F, T125K or T125F or T125W or T125Y, V128L, and T154K or T154Y or T154L or T154W or T154F of native Equus asinus β-lactoglobulin protein (SEQ ID NO: 7). Such two or more amino acid substitutions can comprise or consist of A111P or A111K or A111L or A111Y or A111W or A111F and one or more of: T125K or T125F or T125W or T125Y, V128L, and T154K or T154Y or T154L or T154W or T154F of native Equus asinus β-lactoglobulin protein (SEQ ID NO: 7). Such two or more amino acid substitutions can comprise or consist of T125K or T125F or T125W or T125Y and one or more of: V128L, and T154K or T154Y or T154L or T154W or T154F of native Equus asinus β-lactoglobulin protein (SEQ ID NO: 7). Such two or more amino acid substitutions can comprise or consist of V128L and T154K or T154Y or T154L or T154W or T154F of native Equus asinus β-lactoglobulin protein (SEQ ID NO: 7).

Such two or more amino acid substitutions can comprise or consist of two or more amino acid substitutions selected from the group consisting of: I29L, S30K, S36Y, S36L, S36K, R47L, R47P, T49K, V72L, V72F, V72W, V72Y, D78L, D78Y, D78W, D78F, Y102F, T126K, T126F, T126W, T126Y, V129L, S138R, and combinations thereof of native Equus caballus β-lactoglobulin protein (SEQ ID NO: 8), and corresponding two or more amino acid substitutions in homologs and orthologs of native Equus caballus β-lactoglobulin protein. Such two or more amino acid substitutions can comprise or consist of I29L and one or more of: S30K, S36Y or S36L or S36K, R47L or R47P, T49K, V72L or V72F or V72W or V72Y, D78L or D78Y or D78W or D78F, Y102F, T126K or T126F or T126W or T126Y, V129L, and S138R of native Equus caballus β-lactoglobulin protein (SEQ ID NO: 8). Such two or more amino acid substitutions can comprise or consist of S30K and one or more of: S36Y or S36L or S36K, R47L or R47P, T49K, V72L or V72F or V72W or V72Y, D78L or D78Y or D78W or D78F, Y102F, T126K or TI 26F or T126W or T126Y, V129L, and S138R of native Equus caballus β-lactoglobulin protein (SEQ ID NO: 8). Such two or more amino acid substitutions can comprise or consist of S36Y or S36L or S36K and one or more of: R47L or R47P, T49K, V72L or V72F or V72W or V72Y, D78L or D78Y or D78W or D78F, Y102F, T126K or T126F or T126W or T126Y, V129L, and S138R of native Equus caballus β-lactoglobulin protein (SEQ ID NO: 8). Such two or more amino acid substitutions can comprise or consist of R47L or R47P and one or more of: T49K, V72L or V72F or V72W or V72Y, D78L or D78Y or D78W or D78F, Y102F, T126K or TI 26F or T126W or T126Y, V129L, and S138R of native Equus caballus β-lactoglobulin protein (SEQ ID NO: 8). Such two or more amino acid substitutions can comprise or consist of T49K and one or more of: V72L or V72F or V72W or V72Y, D78L or D78Y or D78W or D78F, Y102F, T126K or T126F or T126W or T126Y, V129L, and S138R of native Equus caballus β-lactoglobulin protein (SEQ ID NO: 8). Such two or more amino acid substitutions can comprise or consist of V72L or V72F or V72W or V72Y and one or more of D78L or D78Y or D78W or D78F, Y102F, T126K or T126F or T126W or T126Y, V129L, and S138R of native Equus caballus β-lactoglobulin protein (SEQ ID NO: 8). Such two or more amino acid substitutions can comprise or consist of D78L or D78Y or D78W or D78F and one or more of: Y102F, T126K or T126F or T126W or T126Y, V129L, and S138R of native Equus caballus β-lactoglobulin protein (SEQ ID NO: 8). Such two or more amino acid substitutions can comprise or consist of Y102F and one or more of: T126K or T126F or T126W or T126Y, V129L, and S138R of native Equus caballus β-lactoglobulin protein (SEQ ID NO: 8). Such two or more amino acid substitutions can comprise or consist of T126K or T126F or T126W or T126Y and one or more of: V129L and S138R of native Equus caballus β-lactoglobulin protein (SEQ ID NO: 8). Such two or more amino acid substitutions can comprise or consist of V129L and S1 38R of native Equus caballus β-lactoglobulin protein (SEQ ID NO: 8).

Such two or more amino acid substitutions can comprise or consist of two or more amino acid substitutions selected from the group consisting of: I29L, S30K, S36Y, S36L, S36K, R47L, R47P, T49K, V72L, V72F, V72W, V72Y, D78L, D78Y, D78W, D78F, Y102F, T126K, T126F, T126W, T126Y, V129L, S138R, H147R, and combinations thereof of native Equus asinus β-lactoglobulin protein (SEQ ID NO: 9), and corresponding two or more amino acid substitutions in homologs and orthologs of native Equus asinus β-lactoglobulin protein. Such two or more amino acid substitutions can comprise or consist of I29L and one or more of: S30K, S36Y or S36L or S36K, R47L or R47P, T49K, V72L or V72F or V72W or V72Y, D78L or D78Y or D78W or D78F, Y102F, T126K or T126F or T126W or T126Y, V129L, S138R, and H147R of native Equus asinus β-lactoglobulin protein (SEQ ID NO: 9). Such two or more amino acid substitutions can comprise or consist of S30K and one or more of: S36Y or S36L or S36K, R47L or R47P, T49K, V72L or V72F or V72W or V72Y, D78L or D78Y or D78W or D78F, Y102F, T126K or T126F or T126W or T126Y, V129L, S138R, and H147R of native Equus asinus β-lactoglobulin protein (SEQ ID NO: 9). Such two or more amino acid substitutions can comprise or consist of S36Y or S36L or S36K and one or more of: R47L or R47P, T49K, V72L or V72F or V72W or V72Y, D78L or D78Y or D78W or D78F, Y102F, T126K or T126F or T126W or T126Y, V129L, S138R, and H147R of native Equus asinus β-lactoglobulin protein (SEQ ID NO: 9). Such two or more amino acid substitutions can comprise or consist of R47L or R47P and one or more of: T49K, V72L or V72F or V72W or V72Y, D78L or D78Y or D78W or D78F, Y102F, T126K or T126F or T126W or T126Y, V129L, S138R, and H147R of native Equus asinusβ-lactoglobulin protein (SEQ ID NO: 9). Such two or more amino acid substitutions can comprise or consist of T49K and one or more of: V72L or V72F or V72W or V72Y, D78L or D78Y or D78W or D78F, Y102F, T126K or T126F or T126W or T126Y, V129L, S138R, and H147R of native Equus asinus β-lactoglobulin protein (SEQ ID NO: 9). Such two or more amino acid substitutions can comprise or consist of V72L or V72F or V72W or V72Y and one or more of: D78L or D78Y or D78W or D78F, Y102F, T126K or T126F or T126W or T126Y, V129L, S138R, and H147R of native Equus asinus β-lactoglobulin protein (SEQ ID NO: 9). Such two or more amino acid substitutions can comprise or consist of D78L or D78Y or D78W or D78F and one or more of: Y102F, T126K or T126F or T126W or T126Y, V129L, S138R, and H147R of native Equus asinus β-lactoglobulin protein (SEQ ID NO: 9). Such two or more amino acid substitutions can comprise or consist of Y102F and one or more of: T126K or T126F or T126W or T126Y, V129L, S138R, and H147R of native Equus asinus β-lactoglobulin protein (SEQ ID NO: 9). Such two or more amino acid substitutions can comprise or consist of T126K or T126F or T126W or T126Y and one or more of: V129L, S138R, and H147R of native Equus asinus l-lactoglobulin protein (SEQ ID NO: 9). Such two or more amino acid substitutions can comprise or consist of V129L and one or more of: S138R and H147R of native Equus asinus β-lactoglobulin protein (SEQ ID NO: 9). Such two or more amino acid substitutions can comprise or consist of S138R and H147R of native Equus asinus β-lactoglobulin protein (SEQ ID NO: 9).

Such two or more amino acid substitutions can comprise or consist of K47L, G52D, and D53N of native Bos taurus β-lactoglobulin protein (SEQ ID NO: 1 or 2), for example such that amino acid sequence KPTPEGD at amino acid position 47 through 53 of native Bos taurus β-lactoglobulin protein (SEQ ID NO: 1 or 2) is converted to amino acid sequence LPTPEDN (e.g., to produce a non-native pepsin recognition or cleavage sequence), Such two or more amino acid substitutions can comprise or consist of I72L, A73G, K77E, 178N, A80K, and V81K of native Bos taurus β-lactoglobulin protein (SEQ ID NO: 1 or 2), for example such that amino acid sequence IAEKTKIPAV at amino acid position 72 through 81 of native Bos taurus β-lactoglobulin protein (SEQ ID NO: 1 or 2) is converted to amino acid sequence LGEKTENPKK (e.g., to produce a non-native pepsin and trypsin recognition or cleavage sequence), Such two or more amino acid substitutions can comprise or consist of K83T, 184V, D85N, A86Y, L87Q, N88G, and N90R of native Bos taurus β-lactoglobulin protein (SEQ ID NO: 1 or 2), for example such that amino acid sequence KIDALNEN at amino acid position 83 through 90 of native Bos taurus β-lactoglobulin protein (SEQ ID NO: 1 or 2) is converted to amino acid sequence TVNYQGER (e.g., to produce a non-native pepsin, chymotrypsin, and trypsin recognition or cleavage sequence), Such two or more amino acid substitutions can comprise or consist of D85N, A86Y, N88D, and N90D of native Bos taurus β-lactoglobulin protein (SEQ ID NO: 1 or 2), for example such that amino acid sequence DALNEN at amino acid position 85 through 90 of native Bos taurus β-lactoglobulin protein (SEQ ID NO: 1 or 2) is converted to amino acid sequence NYLDED (e.g., to produce a non-native pepsin and chymotrypsin recognition or cleavage sequence), Such two or more amino acid substitutions can comprise or consist of E108G, N109P, S110P, A111L, E112P, P113S, E114A, Q115E, S116HG, and L117M of native Bos taurus β-lactoglobulin protein (SEQ ID NO: 1), for example such that amino acid sequence ENSAEPEQSL at amino acid position 108 through 117 of native Bos taurus β-lactoglobulin protein (SEQ ID NO: 1) is converted to amino acid sequence GPPLPSAEHGM (e.g., to produce a non-native pepsin recognition or cleavage sequence), Such two or more amino acid substitutions can comprise or consist of E108G, N109P, S110P, A111L, E112P, P113S, E114A, Q115E, S116HG, L117M, and A118V of native Bos taurus β-lactoglobulin protein (SEQ ID NO: 2), for example such that amino acid sequence ENSAEPEQSLA at amino acid position 108 through 118 of native Bos taurus β-lactoglobulin protein (SEQ ID NO: 2) is converted to amino acid sequence GPPLPSAEHGMV (e.g., to produce a non-native pepsin recognition or cleavage sequence), Such two or more amino acid substitutions can comprise or consist of E127K and D130K of native Bos taurus β-lactoglobulin protein (SEQ ID NO: 1 or 2), for example such that amino acid sequence EVDD at amino acid position 127 through 130 of native Bos taurus β-lactoglobulin protein (SEQ ID NO: 1 or 2) is converted to amino acid sequence KVDK (e.g., to produce a non-native trypsin recognition or cleavage sequence), Such two or more amino acid substitutions can comprise or consist of K141Q, A142P, M145G, and H146R of native Bos taurus β-lactoglobulin protein (SEQ ID NO: 1 or 2), for example such that amino acid sequence KALPMH at amino acid position 141 through 146 of native Bos taurus β-lactoglobulin protein (SEQ ID NO: 1 or 2) is converted to amino acid sequence QPLPGR (e.g., to produce a non-native pepsin and trypsin recognition or cleavage sequence), Such two or more amino acid substitutions can comprise or consist of P153L and Q155R of native Bos taurus β-lactoglobulin protein (SEQ ID NO: 1 or 2), for example such that amino acid sequence PTQ at amino acid position 153 through 155 of native Bos taurus β-lactoglobulin protein (SEQ ID NO: 1 or 2) is converted to amino acid sequence LTR (e.g., to produce a non-native pepsin and trypsin recognition or cleavage sequence), Such two or more amino acid substitutions can comprise or consist of F151L, N152D, P153L, T154K, and L156M of native Bos taurus β-lactoglobulin protein (SEQ ID NO: 1 or 2), for example such that amino acid sequence FNPTQL at amino acid position 151 through 156 of native Bos taurus β-lactoglobulin protein (SEQ ID NO: 1 or 2) is converted to amino acid sequence LDLKQM (e.g., to produce a non-native pepsin and trypsin recognition or cleavage sequence).

Such two or more amino acid substitutions can comprise or consist of K47L and G52D of native Ovis aries musimon or Ovis aries β-lactoglobulin protein (SEQ ID NO: 4 or 5, respectively), for example such that amino acid sequence KPTPEG at amino acid position 47 through 52 of native Ovis aries musimon or Ovis aries β-lactoglobulin protein (SEQ ID NO: 4 or 5, respectively) is converted to amino acid sequence LPTPED (e.g., to produce a non-native pepsin recognition or cleavage sequence), Such two or more amino acid substitutions can comprise or consist of I72L, A73G, K77E, 178N, A80K, and V81K of native Ovis aries musimon or Ovis aries β-lactoglobulin protein (SEQ ID NO: 4 or 5, respectively), for example such that amino acid sequence IAEKTKIPAV at amino acid position 72 through 81 of native Ovis aries musimon or Ovis aries β-lactoglobulin protein (SEQ ID NO: 4 or 5, respectively) is converted to amino acid sequence LGEKTENPKK (e.g., to produce a non-native pepsin and trypsin recognition or cleavage sequence), Such two or more amino acid substitutions can comprise or consist of K83T, 184V, D85N, A86Y, L87Q, N88G, and N90R of native Ovis aries musimon or Ovis aries β-lactoglobulin protein (SEQ ID NO: 4 or 5, respectively), for example such that amino acid sequence KIDALNEN at amino acid position 83 through 90 of native Ovis aries musimon or Ovis aries β-lactoglobulin protein (SEQ ID NO: 4 or 5, respectively) is converted to amino acid sequence TVNYQGER (e.g., to produce a non-native pepsin, chymotrypsin, and trypsin recognition or cleavage sequence), Such two or more amino acid substitutions can comprise or consist of D85N, A86Y, N88D, and N90D of native Ovis aries musimon or Ovis aries β-lactoglobulin protein (SEQ ID NO: 4 or 5, respectively), for example such that amino acid sequence DALNEN at amino acid position 85 through 90 of native Ovis aries musimon or Ovis aries β-lactoglobulin protein (SEQ ID NO: 4 or 5, respectively) is converted to amino acid sequence NYLDED (e.g., to produce a non-native pepsin and chymotrypsin recognition or cleavage sequence), Such two or more amino acid substitutions can comprise or consist of E108G, N109P, S110P, A111L, E112P, P113S, E114A, Q115E, S116HG, L117M, and A118V of native Ovis aries musimon or Ovis aries β-lactoglobulin protein (SEQ ID NO: 4 or 5, respectively), for example such that amino acid sequence ENSAEPEQSLA at amino acid position 108 through 118 of native Ovis aries musimon or Ovis aries β-lactoglobulin protein (SEQ ID NO: 4 or 5, respectively) is converted to amino acid sequence GPPLPSAEHGMV (e.g., to produce a non-native pepsin recognition or cleavage sequence), Such two or more amino acid substitutions can comprise or consist of E127K and N130K of native Ovis aries musimon or Ovis aries β-lactoglobulin protein (SEQ ID NO: 4 or 5, respectively), for example such that amino acid sequence EVDN at amino acid position 127 through 130 of native Ovis aries musimon or Ovis aries β-lactoglobulin protein (SEQ ID NO: 4 or 5, respectively) is converted to amino acid sequence KVDK (e.g., to produce a non-native trypsin recognition or cleavage sequence), Such two or more amino acid substitutions can comprise or consist of K141Q, A142P, M145G, and H146R of native Ovis aries musimon or Ovis aries β-lactoglobulin protein (SEQ ID NO: 4 or 5, respectively), for example such that amino acid sequence KALPMH at amino acid position 141 through 146 of native Ovis aries musimon or Ovis aries β-lactoglobulin protein (SEQ ID NO: 4 or 5, respectively) is converted to amino acid sequence QPLPGR (e.g., to produce a non-native pepsin and trypsin recognition or cleavage sequence), Such two or more amino acid substitutions can comprise or consist of P153L, and Q155R of native Ovis aries musimon or Ovis aries β-lactoglobulin protein (SEQ ID NO: 4 or 5, respectively), for example such that amino acid sequence PTQ at amino acid position 153 through 155 of native Ovis aries musimon or Ovis aries β-lactoglobulin protein (SEQ ID NO: 4 or 5, respectively) is converted to amino acid sequence LTR (e.g., to produce a non-native pepsin and trypsin recognition or cleavage sequence), Such two or more amino acid substitutions can comprise or consist of F151L, N152D, P153L, T154K, and L156M of native Ovis aries musimon or Ovis aries β-lactoglobulin protein (SEQ ID NO: 4 or 5, respectively), for example such that amino acid sequence FNPTQL at amino acid position 151 through 156 of native Ovis aries musimon or Ovis aries β-lactoglobulin protein (SEQ ID NO: 4 or 5, respectively) is converted to amino acid sequence LDLKQM (e.g., to produce a non-native pepsin and trypsin recognition or cleavage sequence).

Such two or more amino acid substitutions can comprise or consist of F72L, A73G, S78N, A80K, and E81K of native Equus caballus or Equus asinus β-lactoglobulin protein (SEQ ID NO: 6 or 7, respectively), for example such that amino acid sequence FAEKTESPAE at amino acid position 72 through 81 of native Equus caballus β-lactoglobulin protein (SEQ ID NO: 6 or 7, respectively) is converted to amino acid sequence LGEKTENPKK (e.g., to produce a non-native pepsin and trypsin recognition or cleavage sequence), Such two or more amino acid substitutions can comprise or consist of K108G, N109P, A110P, A111L, T112P, P113S, G14A, Q115E, S116HG, and L117M of native Equus caballus β-lactoglobulin protein (SEQ ID NO: 6 or 7, respectively), for example such that amino acid sequence KNAATPGQSL at amino acid position 108 through 117 of native Equus caballus β-lactoglobulin protein (SEQ ID NO: 6 or 7, respectively) is converted to amino acid sequence GPPLPSAEHGM (e.g., to produce a non-native pepsin recognition or cleavage sequence), Such two or more amino acid substitutions can comprise or consist of P151L, T154K, and R155Q of native Equus caballus β-lactoglobulin protein (SEQ ID NO: 6 or 7, respectively), for example such that amino acid sequence PDLTR at amino acid position 151 through 155 of native Equus caballus β-lactoglobulin protein (SEQ ID NO: 6 or 7, respectively) is converted to amino acid sequence LDLKQ (e.g., to produce a non-native pepsin and trypsin recognition or cleavage sequence).

Such two or more amino acid substitutions can comprise or consist of R47L and G52D of native Equus caballus or Equus asinus β-lactoglobulin protein (SEQ ID NO: 8 or 9, respectively), for example such that amino acid sequence RPTPEG at amino acid position 47 through 52 of native Equus caballus or Equus asinus β-lactoglobulin protein (SEQ ID NO: 8 or 9, respectively) is converted to amino acid sequence LPTPED (e.g., to produce a non-native pepsin recognition or cleavage sequence), Such two or more amino acid substitutions can comprise or consist of V72L, A73G, Q74E, D78N, A80K, and V81K of native Equus caballus or Equus asinus β-lactoglobulin protein (SEQ ID NO: 8 or 9, respectively), for example such that amino acid sequence VAQKTEDPAV at amino acid position 72 through 81 of native Equus caballus or Equus asinus β-lactoglobulin protein (SEQ ID NO: 8 or 9, respectively) is converted to amino acid sequence LGEKTENPKK (e.g., to produce a non-native pepsin and trypsin recognition or cleavage sequence), Such two or more amino acid substitutions can comprise or consist of P154L, S155T, and G156R of native Equus caballus or Equus asinus β-lactoglobulin protein (SEQ ID NO: 8 or 9, respectively), for example such that amino acid sequence PSG at amino acid position 154 through 156 of native Equus caballus or Equus asinus β-lactoglobulin protein (SEQ ID NO: 8 or 9, respectively) is converted to amino acid sequence LTR (e.g., to produce a non-native pepsin and trypsin recognition or cleavage sequence), Such two or more amino acid substitutions can comprise or consist of Q152L, P154L, S155K, G156Q, and G157M of native Equus caballus or Equus asinus β-lactoglobulin protein (SEQ ID NO: 8 or 9, respectively), for example such that amino acid sequence QDPSGG at amino acid position 152 through 157 of native Equus caballus or Equus asinus β-lactoglobulin protein (SEQ ID NO: 8 or 9, respectively) is converted to amino acid sequence LDLKQM (e.g., to produce a non-native pepsin and trypsin recognition or cleavage sequence).

Such two or more amino acid substitutions can comprise or consist of K47L and G52D of native Capra hircus β-lactoglobulin protein (SEQ ID NO: 10), for example such that amino acid sequence KPTPEG at amino acid position 47 through 52 of native Capra hircus β-lactoglobulin protein (SEQ ID NO: 10) is converted to amino acid sequence LPTPED (e.g., to produce a non-native pepsin recognition or cleavage sequence), Such two or more amino acid substitutions can comprise or consist of I72L, A73G, K77E, 178N, A80K, and V81K of native Capra hircus β-lactoglobulin protein (SEQ ID NO: 10), for example such that amino acid sequence IAEKTKIPAV at amino acid position 72 through 81 of native Capra hircus β-lactoglobulin protein (SEQ ID NO: 10) is converted to amino acid sequence LGEKTENPKK (e.g., to produce a non-native pepsin and trypsin recognition or cleavage sequence), Such two or more amino acid substitutions can comprise or consist of K83T, 184V, D85N, A86Y, L87Q, N88G, and N90R of native Capra hircus β-lactoglobulin protein (SEQ ID NO: 10), for example such that amino acid sequence KIDALNEN at amino acid position 83 through 90 of native Capra hircus β-lactoglobulin protein (SEQ ID NO: 10) is converted to amino acid sequence TVNYQGER (e.g., to produce a non-native pepsin, chymotrypsin, and trypsin recognition or cleavage sequence), Such two or more amino acid substitutions can comprise or consist of D85N, A86Y, N88D, and N90D of native Capra hircus β-lactoglobulin protein (SEQ ID NO: 10), for example such that amino acid sequence DALNEN at amino acid position 85 through 90 of native Capra hircus β-lactoglobulin protein (SEQ ID NO: 10) is converted to amino acid sequence NYLDED (e.g., to produce a non-native pepsin and chymotrypsin recognition or cleavage sequence), Such two or more amino acid substitutions can comprise or consist of E108G, N109P, S110P, A111L, E112P, P113S, E114A, Q115E, S116HG, L117M, and A118V of native Capra hircus β-lactoglobulin protein (SEQ ID NO: 10), for example such that amino acid sequence ENSAEPEQSLA at amino acid position 108 through 118 of native Capra hircus β-lactoglobulin protein (SEQ ID NO: 10) is converted to amino acid sequence GPPLPSAEHGMV (e.g., to produce a non-native pepsin recognition or cleavage sequence), Such two or more amino acid substitutions can comprise or consist of K141Q, A142P, M145G, and H146R of native Capra hircus β-lactoglobulin protein (SEQ ID NO: 10), for example such that amino acid sequence KALPMH at amino acid position 141 through 146 of native Capra hircus β-lactoglobulin protein (SEQ ID NO: 10) is converted to amino acid sequence QPLPGR (e.g., to produce a non-native pepsin and trypsin recognition or cleavage sequence), Such two or more amino acid substitutions can comprise or consist of P153L and Q155R of native Capra hircus β-lactoglobulin protein (SEQ ID NO: 10), for example such that amino acid sequence PTQ at amino acid position 153 through 155 of native Capra hircus β-lactoglobulin protein (SEQ ID NO: 10) is converted to amino acid sequence LTR (e.g., to produce a non-native pepsin and trypsin recognition or cleavage sequence), Such two or more amino acid substitutions can comprise or consist of F151L, N152D, P153L, T154K, and L156M of native Capra hircus β-lactoglobulin protein (SEQ ID NO: 10), for example such that amino acid sequence FNPTQL at amino acid position 151 through 156 of native Capra hircus β-lactoglobulin protein (SEQ ID NO: 10) is converted to amino acid sequence LDLKQM (e.g., to produce a non-native pepsin and trypsin recognition or cleavage sequence).

The modification that attenuates or essentially eliminates allergenicity of the recombinant β-lactoglobulin protein according to any of the above can be a modification that eliminates an allergenic epitope comprised in a native β-lactoglobulin protein. Non-limiting examples of modifications that eliminate an allergenic epitope include one or more amino acid substitutions selected from the group consisting of: A111G, A111I, A111L, A111V, V118G, V118I, V118L, A132G, A132I, A132L, A132V, A139G, A139I, A139L, A139V, A142G, A142I, A142L, A142V, A16G, A16I, A16L, A16V, A23C, A23G, A23H, A23K, A23P, A23W, A25C, A25D, A25E, A25G, A25P, A25W, A26C, A26D, A26E, A26G, A26P, A26W, A34G, A34I, A34L, A34V, A37G, A37I, A37L, A37V, A67G, A67I, A67L, A67V, A73G, A73I, A73L, A73V, A80G, A80I, A80L, A80V, A86G, A86I, A86L, A86Q, A86V, D11E, D11N, D11Q, D129E, D129N, D129Q, D130E, D130N, D130Q, D137E, D137N, D137Q, D33E, D33N, D33Q, D53E, D53N, D53Q, D85E, D85N, D85Q, D98E, D98N, D98Q, E108D, E108N, E108Q, E112D, E112N, E112Q, E114D, E114N, E114Q, E127D, E127N, E127Q, E131D, E131N, E131Q, E134D, E134N, E134Q, E157C, E157D, E157G, E157H, E157N, E157P, E157Q, E158D, E158N, E158Q, E44D, E44G, E44N, E44Q, E45D, E45N, E45Q, E51D, E51G, E51N, E51, E55D, E55N, E55Q, E62D, E62N, E62Q, E65D, E65N, E65Q, E74D, E74N, E74Q, F105W, F105Y, F136W, F136Y, F151A, F151C, F151D, F151E, F151G, F151H, F151I, F151K, F151N, F151P, F151Q, F151R, F151S, F151T, F151V, F151W, F151Y, F82W, F82Y, G52A, G52I, G52L, G52V, D64A, D64I, D64L, D64V, G9A, G9I, G9L, G9V, H146K, H146P, H146R, H161K, H161R, 112A, 112G, I12L, I12V, I147A, I147C, I147D, I147E, I147G, I147H, I147K, I147L, I147N, I147P, I147Q, I147R, I147S, I147T, I147V, I162A, I162G, I162L, I162V, 129C, 129D, 129E, 129G, 129H, 129K, 129N, 129P, 129R, 12A, 12G, 12L, 12V, 156A, 156G, 156L, 156V, 171A, 171G, 171L, 171V, I72A, 172G, I72L, 172V, I78A, 178G, I78L, I78V, 184A, I84G, 184L, 184V, K100H, K101H, K47H, K47R, K75H, K77H, K83H, K8H, K8R, L103A, L103G, L103I, L103V, L104A, L104G, L104I, L104V, L10A, L10G, L10I, L10V, L117A, L117G, L117I, L117V, L122A, L122G, L122I, L122V, L133A, L133G, L133I, L133V, L140A, L140G, L140I, L140V, L143A, L143G, L143I, L143V, L149A, L149C, L149D, L149E, L149G, L149H, L149I, L149K, L149N, L149P, L149Q, L149R, L149S, L149T, L149V, L156A, L156C, L156D, L156E, L156G, L156H, L156I, L156K, L156M, L156N, L156P, L156Q, L156R, L156S, L156T, L156V, L1A, L1G, L1I, L1V, L22C, L22D, L22E, L22G, L22H, L22K, L22N, L22P, L22Q, L22R, L22S, L22T, L22W, L31A, L31C, L31D, L31E, L31G, L31H, L31I, L31K, L31M, L31N, L31P, L31Q, L31R, L31S, L31T, L31V, L31W, L32A, L32C, L32D, L32E, L32G, L32H, L32I, L32K, L32M, L32N, L32P, L32Q, L32R, L32T, L32V, L32W, L39A, L39G, L39I, L39V, L46A, L46G, L46I, L46V, L54A, L54G, L54I, L54V, L57A, L57G, L57I, L57V, L58A, L58G, L58I, L58V, M107S, M107T, M145S, M145T, M24A, M24C, M24D, M24E, M24G, M24N, M24P, M24Q, M24S, M7S, M7T, N109D, N109E, N109Q, N152C, N152D, N152E, N152G, N152Q, N63D, N63E, N63Q, P48G, P50G, Q115D, Q115E, Q115N, Q120D, Q120E, Q120N, Q13D, Q13E, Q13N, Q155D, Q155E, Q155N, Q159D, Q159E, Q159N, Q35D, Q35E, Q35N, Q59D, Q59E, Q59N, Q5D, Q5E, Q5N, Q68D, Q68E, Q68N, R124H, R40H, S110M, S110T, S116M, S116T, S150C, S150D, S150E, S150G, S150M, S150N, S150T, S21D, S21G, S27C, S27D, S27G, S30C, S30D, S30G, S36M, S36T, T125M, T125S, T154G, T154H, T154M, T154S, T49G, T49M, T49S, T4M, T4S, T6M, T6S, T76M, T76S, T97M, T97S, V123A, V123G, V123I, V123L, V128A, V128G, V128I, V128L, V15A, V15G, V15I, V15L, V3A, V3G, V3I, V3L, V41A, V41G, V41I, V41L, V43A, V43G, V43I, V43L, V81A, V81G, V81I, V81L, W19C, W19D, W19E, W19F, W19G, W19H, W19K, W19N, W19P, W19Q, W19R, W19S, W19T, W19Y, W61F, W61Y, Y20A, Y20C, Y20D, Y20E, Y20G, Y20H, Y20M, Y20N, Y20Q, Y20R, Y20S, Y20T, Y20V, Y42G, and combinations thereof of native Bos taurus β-lactoglobulin protein (SEQ ID NO: 1), and corresponding amino acid substitutions in homologs and orthologs of native Bos taurus β-lactoglobulin protein.

The modification that attenuates or essentially eliminates allergenicity of the recombinant β-lactoglobulin protein according to any of the above can be a modification that eliminates an allergenic epitope comprised in a native β-lactoglobulin protein. Non-limiting examples of modifications that eliminate an allergenic epitope include one or more amino acid substitutions selected from the group consisting of: A111G, A111I, A111L, A111V, A118G, A118I, A118L, A118V, A132G, A132I, A132L, A132V, A139G, A139I, A139L, A139V, A142G, A142I, A142L, A142V, A16G, A16I, A16L, A16V, A23C, A23G, A23H, A23K, A23P, A23W, A25C, A25D, A25E, A25G, A25P, A25W, A26C, A26D, A26E, A26G, A26P, A26W, A34G, A34I, A34L, A34V, A37G, A37I, A37L, A37V, A67G, A67I, A67L, A67V, A73G, A73I, A73L, A73V, A80G, A80I, A80L, A80V, A86G, A86I, A86L, A86Q, A86V, D11E, D11N, D11Q, D129E, D129N, D129Q, D130E, D130N, D130Q, D137E, D137N, D137Q, D33E, D33N, D33Q, D53E, D53N, D53Q, D85E, D85N, D85Q, D98E, D98N, D98Q, E108D, E108N, E108Q, E112D, E112N, E112Q, E114D, E114N, E114Q, E127D, E127N, E127Q, E131D, E131N, E131Q, E134D, E134N, E134Q, E157C, E157D, E157G, E157H, E157N, E157P, E157Q, E158D, E158N, E158Q, E44D, E44G, E44N, E44Q, E45D, E45N, E45Q, E51D, E51G, E51N, E51Q, E55D, E55N, E55Q, E62D, E62N, E62Q, E65D, E65N, E65Q, E74D, E74N, E74Q, F105W, F105Y, F136W, F136Y, F151A, F151C, F151D, F151E, F151G, F151H, F151I, F151K, F151N, F151P, F151Q, F151R, F151S, F151T, F151V, F151W, F151Y, F82W, F82Y, G52A, G52I, G52L, G52V, G64A, G64I, G64L, G64V, G9A, G9I, G9L, G9V, H146K, H146P, H146R, H161K, H161R, 112A, I12G, I12L, I12V, I147A, I147C, I147D, I147E, I147G, I147H, I147K, I147L, I147N, I147P, I147Q, I147R, I147S, I147T, I147V, I162A, I162G, I162L, I162V, 129C, 129D, 129E, 129G, 129H, 129K, 129N, 129P, 129R, 12A, 12G, 12L, 12V, 156A, 156G, 156L, 156V, 171A, 171G, 171L, I71V, I72A, I72G, I72L, I72V, I78A, I78G, I78L, 178V, 184A, 184G, I84L, 184V, K100H, K101H, K47H, K47R, K75H, K77H, K83H, K8H, K8R, L103A, L103G, L103I, L103V, L104A, L104G, L104I, L104V, L10A, L10G, L10I, L10V, L117A, L117G, L117I, L117V, L122A, L122G, L122I, L122V, L133A, L133G, L133I, L133V, L140A, L140G, L140I, L140V, L143A, L143G, L143I, L143V, L149A, L149C, L149D, L149E, L149G, L149H, L149I, L149K, L149N, L149P, L149Q, L149R, 149S, L149T, L149V, 156A, L156C, L156D, L156E, L156G, L156H, L156I, L156K, L156M, L156N, L156P, L156Q, L156R, L156S, L156T, L156V, L1A, L1G, L1I, L1V, L22C, L22D, L22E, L22G, L22H, L22K, L22N, L22P, L22Q, L22R, L22S, L22T, L22W, L31A, L3I C, L31 D, L31E, L31G, L31H, L31I, L31K, L31M, L31N, L31P, L31Q, L31R, L31S, L31T, L31V, L31W, L32A, L32C, L32D, L32E, L32G, L32H, L32I, L32K, L32M, L32N, L32P, L32Q, L32R, L32T, L32V, L32W, L39A, L39G, L39I, L39V, L46A, L46G, L46I, L46V, L54A, L54G, L54I, L54V, L57A, L57G, L57I, L57V, L58A, L58G, L58I, L58V, M107S, M107T, M145S, M145T, M24A, M24C, M24D, M24E, M24G, M24N, M24P, M24Q, M24S, M7S, M7T, N109D, N109E, N109Q, N152C, N152D, N152E, N152G, N152Q, N63D, N63E, N63Q, P48G, P50G, Q115D, Q115E, Q115N, Q120D, Q120E, Q120N, Q13D, Q13E, Q13N, Q155D, Q155E, Q155N, Q159D, Q159E, Q159N, Q35D, Q35E, Q35N, Q59D, Q59E, Q59N, Q5D, Q5E, Q5N, Q68D, Q68E, Q68N, R124H, R40H, S110M, S110T, S116M, S116T, S150C, S150D, S150E, S150G, S150M, S150N, S150T, S21D, S21G, S27C, S27D, S27G, S30C, S30D, S30G, S36M, S36T, T125M, T125S, T154G, T154H, T154M, T154S, T49G, T49M, T49S, T4M, T4S, T6M, T6S, T76M, T76S, T97M, T97S, V123A, V123G, V123I, V123L, V128A, V128G, V128I, V128L, V15A, V15G, V15I, V15L, V3A, V3G, V3I, V3L, V41A, V41G, V41I, V41L, V43A, V43G, V43I, V43L, V81A, V81G, V81I, V81L, W19C, W19D, W19E, W19F, W19G, W19H, W19K, W19N, W19P, W19Q, W19R, W19S, W19T, W19Y, W61F, W61Y, Y20A, Y20C, Y20D, Y20E, Y20G, Y20H, Y20M, Y20N, Y20Q, Y20R, Y20S, Y20T, Y20V, Y42G, and combinations thereof of native Bos taurus β-lactoglobulin protein (SEQ ID NO: 2), and corresponding amino acid substitutions in homologs and orthologs of native Bos taurus β-lactoglobulin protein.

### Recombinant α-Lactalbumin Protein

The recombinant milk protein having attenuated or essentially eliminated allergenicity according to any of the above can be a recombinant α-lactalbumin protein.

The modification that attenuates or essentially eliminates allergenicity of the recombinant α-lactalbumin protein according to any of the above can be a modification that introduces a non-native protease recognition or cleavage sequence in or in the vicinity (e.g., within 1, 2, 3, 4, 5, 6, 7, 8, 9, 10, 11, 12, 13, 14, or 15 amino acids) of a solvent-exposed region of a corresponding native α-lactalbumin protein (e.g., a Bos taurus α-lactalbumin protein). Non-limiting examples of solvent-exposed regions include regions spanning from amino acid 5 to amino acid 18 of native Bos taurus α-lactalbumin protein (SEQ ID NO: 3), and corresponding regions in homologs and orthologs of native Bos taurus α-lactalbumin protein.

The modification that attenuates or essentially eliminates allergenicity of the recombinant α-lactalbumin protein according to any of the above can be a modification that introduces a non-native protease recognition or cleavage sequence in or in the vicinity (e.g., within 1, 2, 3, 4, 5, 6, 7, 8, 9, 10, 11, 12, 13, 14, or 15 amino acids) of an allergenic epitope comprised in a corresponding native α-lactalbumin protein (e.g., a Bos taurus α-lactalbumin protein). Non-limiting examples of allergenic epitopes (e.g., T-cell antigenic epitopes, B-cell antigenic epitopes) include regions spanning from amino acid 1 to amino acid 16, amino acid 13 to amino acid 26, amino acid 47 to amino acid 58, or amino acid 93 to amino acid 102 of native Bos taurus α-lactalbumin protein (SEQ ID NO: 3), and corresponding regions in homologs and orthologs of native Bos taurus α-lactalbumin protein.

The modification that attenuates or essentially eliminates allergenicity of the recombinant α-lactalbumin protein according to any of the above can be a modification that eliminates an allergenic epitope comprised in a native α-lactalbumin protein. Non-limiting examples of modifications that eliminate an allergenic epitope include one or more amino acid substitutions selected from the group consisting of: E1D, E1N, E1Q, Q2D, Q2E, Q2N, L3G, L3A, L3V, L31, T4S, T4M, K5H, K5R, E7D, E7N, E7Q, V8G, V8A, V8L, V81, F9Y, F9W, R10H, R10K, E11D, E11N, E11Q, L12G, L12A, L12V, L12I, K13H, K13R, D14E, D14N, D14Q, L15G, L15A, L15V, L15I, K16H, K16R, G17A, G17V, G17L, G17I, Y18F, Y18W, G19A, G19V, G19L, G19I, G20A, G20V, G20L, G20I, V21G, V21A, V21L, V21I, S22T, S22M, L23G, L23A, L23V, L23I, E25D, E25N, E25Q, W26F, W26Y, S47T, S47M, T48S, T48M, E49D, E49N, E49Q, Y50F, Y50W, G51A, G51V, G51L, G51I, L52G, L52A, L52V, L52I, F53Y, F53W, Q54D, Q54E, Q54N, I55G, I55A, I55V, I55L, N56D, N56E, N56Q, N57D, N57E, N57Q, K58H, K58R, K93H, K93R, K94H, K94R, I95G, I95A, I95V, I95L, L96G, L96A, L96V, L96I, D97E, D97N, D97Q, K98H, K98R, V99G, V99A, V99L, V99I, G100A, G100V, G100L, G100I, I101G, I101A, I101V, I101L, N102D, N102E, N102Q, A109G, A109V, A109L, A109I, L110G, L110A, L110V, L110I, S112T, S112M, E113D, E113N, E113Q, K114H, K114R, L115G, L115A, L115V, L115I, D116E, D116N, D116Q, Q117D, Q117E, Q117N, W118F, W118Y, L119G, L119A, L119V, L191I, E121D, E121N, E121Q, K122H, K122R, L123G, L123A, L123V, AND L123I, and combinations thereof of native Bos taurus α-lactalbumin protein (SEQ ID NO: 3), and corresponding amino acid substitutions in homologs and orthologs of native Bos taurus α-lactalbumin protein.

### Other Effects of Modification

The modifications that attenuates or essentially eliminates allergenicity of the recombinant milk protein according to any of the above can eliminate a post-translational modification (PTM) from the recombinant milk protein that is comprised in a corresponding native milk protein. The term “post-translational modification”, or its acronym “PTM”, as used herein refers to the covalent attachment of a chemical group to a polypeptide after biosynthesis. PTM can occur on the amino acid side chain of the polypeptide or at its C- or N-termini. Non-limiting examples of PTMs include glycosylation (i.e., covalent attachment to proteins of glycan groups (e.g., monosaccharides, disaccharides, polysaccharides, linear glycans, branched glycans, glycans with galf residues, glycans with sulfate and/or phosphate residues, D-glucose, D-galactose, D-mannose, L-fucose, N-acetyl-D-galactose amine, N-acetyl-D-glucose amine, N-acetyl-D-neuraminic acid, galactofuranose, phosphodiesters, N-acetylglucosamine, N-acetylgalactosamine, sialic acid, and combinations thereof; see, for example, Deshpande et al. 2008. Glycobiology 18(8):626) via C-linkage (i.e., C-glycosylation), N-linkage (i.e., N-glycosylation), or O-linkage (i.e., O-glycosylation), or via glypiation (i.e., addition of a glycosylphosphatidylinositol anchor) or phosphoglycosylation (i.e., linked through the phosphate of a phospho-serine)), phosphorylation (i.e., covalent attachment to proteins of phosphate groups), alkylation (i.e., covalent attachment to proteins of alkane groups (e.g., methane group in methylation)), lipidation (i.e., covalent attachment of a lipid group (e.g., isoprenoid group in prenylation and isoprenylation (e.g., farnesol group in farnesylation, geraniol group in geranylation, geranylgeraniol group in geranylgeranylation), fatty acid group in fatty acylation (e.g., myristic acid in myristoylation, palmitic acid in palmitoylation), glycosylphosphatidylinositol anchor in glypiation)), hydroxylation (i.e., covalent attachment of a hydroxide group), sumoylation (i.e., attachment to proteins of Small Ubiquitin-like Modifier (or SUMO) protein), nitrosylation (i.e., attachment to proteins of an NO group; e.g., S-nitrosylation), nitrosothiolation (i.e., attachment to a cysteine thiol in a protein of an NO group to form an S-nitrosothiol)), S-glutathionylation (i.e., attachment to a cysteine thiol in a protein of a glutathione group), and tyrosine nitration (i.e., attachment to tyrosine residues of proteins of nitrate groups). By eliminating a PTM in the recombinant milk protein, the modification can make a protease recognition or cleavage sequence (e.g., a native protease recognition or cleavage sequences, or a non-native protease recognition or cleavage sequences [e.g., non-native protease recognition or cleavage sequences according to any of the above])) more accessible to a protease comprised in a gastrointestinal tract of a mammal (e.g., a human) such that the protease can cleave the recombinant milk protein and thereby attenuate or essentially eliminate allergenicity of the recombinant milk protein.

The modifications that attenuates or essentially eliminates allergenicity of the recombinant milk protein according to any of the above can decrease stability of a protein structure of the recombinant milk protein at an acidic pH (e.g., pH of less than 7, less than 6.5, less than 6, less than 5.5, less than 5, less than 4.5, less than 4, less than 3.5, less than 3, less than 2.5, less than 2, less than 1.5, or less than 1) compared to that of a corresponding native milk protein. By decreasing stability of a protein structure of the recombinant milk protein, the modification can effect solvent exposure of a protease recognition or cleavage sequence (e.g., a native protease recognition or cleavage sequences, or a anon-native protease recognition or cleavage sequences [e.g., a non-native protease recognition or cleavage sequences according to any of the above])), such that a protease comprised in a gastrointestinal tract of a mammal (e.g., a human) can cleave the recombinant milk protein and thereby attenuate or essentially eliminate allergenicity of the recombinant milk protein.

### Recombinant Host Cell

In another aspect, provided herein is a recombinant host cell that is capable of producing the recombinant milk protein according to any of the above (i.e., that comprises a polynucleotide that encodes the recombinant milk protein according to any of the above), wherein the recombinant host cell comprises a recombinant expression construct according to any of the below.

The recombinant expression construct can consist of a single recombinant expression construct, or consist of two or more recombinant expression constructs. In embodiments in which the recombinant expression construct consists of two or more recombinant expression constructs, the two or more recombinant expression constructs can be identical, or at least two of the two or more recombinant expression constructs can differ from each other (e.g., in a promoter sequence, a protein coding sequence, a secretion signal sequence, a termination sequence, and/or an additional regulatory element). The recombinant expression construct can be stably integrated within the genome of the recombinant host cell (e.g., via targeted integration (e.g., via homologous recombination) or random (i.e., non-targeted) integration), or can be not stably integrated but rather maintained extrachromosomally (e.g., on an autonomously replicating recombinant vector provided herein). The recombinant expression construct can consist of two or more recombinant expression constructs, wherein at least one recombinant expression construct is stably integrated within the genome of the recombinant host cell, and at least one recombinant expression construct is not stably integrated.

### Recombinant Expression Construct

In another aspect, provided herein is a recombinant expression construct that is useful for producing the recombinant host cell according to any of the above.

The recombinant expression construct consists of a polynucleotide that comprises one or more expression cassettes, wherein each expression cassette comprises:
a promoter sequence (e.g., a polynucleotide sequence for any of the promoters disclosed herein),
an optional secretion signal sequence (i.e., a sequence that encodes a peptide that mediates the delivery of a nascent protein attached to the peptide to the exterior of the cell in which the nascent protein is synthesized; e.g., a polynucleotide sequence encoding any of the secretion signals disclosed herein),
a milk protein coding sequence (i.e., a polynucleotide sequence encoding the recombinant milk protein according to any of the above), and
a termination sequence (e.g., a polynucleotide sequence for any of the terminators disclosed herein);
   wherein:
the promoter sequence is operably linked in sense orientation to the optional secretion signal sequence and the milk protein coding sequence (i.e., the promoter sequence and the optional secretion signal sequence and the milk protein coding sequence are positioned such that the promoter sequence is effective in mediating or regulating transcription of the optional secretion signal sequence and the milk protein coding sequence).
the optional secretion signal sequence is operably linked in sense orientation to the milk protein coding sequence (i.e., the optional secretion signal sequence and the milk protein coding sequence are positioned such that transcription and translation produces a recombinant milk protein comprising the optional secretion signal), and
the one or more terminator sequences are operably linked to the milk protein coding sequence (i.e., the milk protein coding sequence and the one or more terminator sequences are positioned such that the one or more terminator sequences are effective in terminating transcription of the milk protein coding sequence).

The recombinant expression construct can further comprise sequences for integration by homologous (i.e., targeted integration) or nonhomologous recombination into the genome of a host cell. The recombinant expression construct can comprise at least 10, at least 25, at least 50, at least 100, at least 250, at least 500, at least 750, at least 1,000, or at least 10,000 base pairs that have sufficient identity with a target sequence in the genome of the host cell to enhance the probability of homologous recombination of the recombinant expression construct. Such homologous sequence may be non-coding or coding.

The optional secretion signal sequence and/or milk protein coding sequence comprised in the recombinant expression construct according to any of the above can be codon-optimized for expression in the recombinant host cell according to any of the above.

The recombinant expression construct according to any of the above can be isolated and/or purified.

The recombinant expression construct according to any of the above can be generated upon integration of a fragment of the recombinant expression construct into the genome of a host cell (e.g., the genome of the recombinant host cell according to any of the above). For example, a polynucleotide comprising a milk protein coding sequence (optionally operably linked to a secretion signal sequence) can be stably integrated within the genome of a host cell such that one or more regulatory elements of an endogenous gene locus become operably linked to the milk protein coding sequence, thereby generating the recombinant expression construct according to any of the above.

### Promoter Sequence

The recombinant expression construct according to any of the above can comprise any promoter sequence that is active in the recombinant host cell according to any of the above.

The promoter sequence can be a constitutive promoter sequence (i.e., a promoter sequence that is active under most environmental and developmental conditions), or an inducible or repressible promoter sequence (i.e., a promoter sequence that is active only under certain environmental or developmental conditions [e.g., in presence or absence of certain factors, such as, but not limited to, carbon (e.g., glucose, galactose, lactose, sucrose, cellulose, sophorose, gentiobiose, sorbose, disaccharides that induce the cellulase promoters, starch, tryptophan, thiamine, methanol), phosphate, nitrogen, or other nutrient; temperature; pH; osmolarity; heavy metals or heavy metal ions; inhibitors; stress; catabolites; and combinations thereof]).

The promoter sequence can consist of a single promoter sequence, or of two or more promoter sequences (e.g., combination of two or more promoters or functional parts thereof arranged in sequence, combination of an inducible and a constitutive promoter). The two or more promoter sequences can be identical, or at least two of the two or more promoter sequences cannot be identical.

The promoter sequence can comprise or consist of a bidirectional promoter sequence (i.e., a polynucleotide that initiates transcription in both orientations by recruiting transcription factors, for example generated by fusing two identical or different promoters in opposite directions).

Non-limiting examples of suitable promoter sequences include promoter sequences that are functional in a bacterial host cell from which the recombinant host cell according to any of the above is derived, including T7 promoter, T5 promoter, Tac promoter, pL/pR promoter, phoA promoter, lacUV5 promoter, trc promoter, trp promoter, cstA promoter, xylA promoter, manP promoter, malA promoter, lacA promoter, aprE promoter, AaprE promoter, srfA promoter, p43 promoter, ylbA promoter, aB promoter, veg promoter, PG1 promoter, PG6 promoter, XPL promoter, XPR promoter, and spa promoter, and functional parts and combinations thereof.

Non-limiting examples of suitable promoter sequences include promoter sequences that are functional in a fungal host cell from which the recombinant fungal host cell according to any of the above is derived, including xlnA promoter, xyn1 promoter, xyn2 promoter, xyn3 promoter, xyn4 promoter, bxl1 promoter, cbh1 promoter, cbh2 promoter, egl1 promoter, egl2 promoter, egl3 promoter, egl4 promoter, egl5 promoter, glaA promoter, agdA promoter, gpdA promoter, gpd1 promoter, AOX1 promoter, GAP1 promoter, MET3 promoter, ENO1 promoter, GPD1 promoter, PDC1 promoter, TEF1 promoter, AXE1 promoter, CIP1 promoter, GH61 promoter, PKI1 promoter, RP2 promoter, ADH1 promoter, CUP1 promoter, GAL1 promoter, PGK1 promoter, YPT1 promoter, LAC4 promoter, LAC4-PBI promoter, FLD1 promoter, MOX promoter, DAS1 promoter, DAS2 promoter, GAP1 promoter, STR3 promoter, ADH3 promoter, GUT2 promoter, CYC1 promoter, TDH3 promoter, PGL1 promoter, ADH2 promoter, HXT7 promoter, CLB1 promoter, and PHO5 promoter, and functional parts and combinations thereof.

### Secretion Signal Sequence

The recombinant expression construct according to any of the above can optionally comprise any secretion signal sequence that is active in the recombinant host cell according to any of the above.

The optional secretion signal sequence can encode a secretion signal that mediates translocation of the nascent recombinant milk protein into the ER post-translationally (i.e., protein synthesis precedes translocation such that the nascent recombinant milk protein is present in the cell cytosol prior to translocating into the ER) or co-translationally (i.e., protein synthesis and translocation into the ER occur simultaneously).

Non-limiting examples of suitable secretion signal sequences include secretion signal sequences that are functional in a bacterial host cell from which the recombinant host cell according to any of the above is derived, including secretion signal sequences of genes encoding any of the following proteins: PelB, OmpA, Bla, PhoA, PhoS, MalE, LivK, LivJ, MglB, AraF, AmpC, RbsB, MerP, CpdB, Lpp, LamB, OmpC, PhoE, OmpF, TolC, BtuB, and LutA, and functional parts and combinations thereof.

Non-limiting examples of suitable secretion signal sequences include secretion signal sequences that are functional in a fungal host cell from which the recombinant host cell according to any of the above is derived, including secretion signal sequences of genes encoding any of the following proteins: CBH1, CBH2, EGL1, EGL2, XYN1, XYN2, BXL1, HFB1, HFB2, GLAA, AMYA, AMYC, AAMA, alpha mating factor, SUC2, PHO5, INV, AMY, LIP, PIR, OSTI, and β-glucosidase, and functional parts and combinations thereof.

### Termination Sequence

The recombinant expression construct according to any of the above can comprise any termination sequence that is active in the recombinant host cell according to any of the above.

Non-limiting examples of suitable termination sequences include termination sequences that are functional in a host cell from which the recombinant host cell according to any of the above is derived, including termination sequences of the adh1, amaA, amdS, amyA, aox1, cbh1, cbh2, cyc1, egl1, egl2, gal1, gap1, glaA, gpd1, gpdA, pdc1, pgk1 tef1, tps1, trpC, xyn1, xyn2, xyn3, and xyn4 genes, and functional parts and combinations thereof.

The termination sequence can consist of a single termination sequence, or of two or more termination sequences, wherein the two or more termination sequences can be identical, or at least two of the two or more termination sequences can be not identical. The termination sequence can consist of a bidirectional termination sequence.

### Additional Regulatory Elements

The recombinant expression construct according to any of the above can further comprise additional regulatory elements.

Non-limiting examples of regulatory elements include promoter sequences, termination sequences, transcriptional start sequences, translational start sequences, translation stop sequences, enhancer sequences, activator sequences, response elements, protein recognition sites, inducible elements, protein binding sequences, 5′ and 3′ untranslated regions, upstream activation sequences (UAS), introns, operators (i.e., sequences of nucleic acids adjacent to a promoter that comprise a protein-binding domain where a repressor protein can bind and reduce or eliminate activity of the promoter), efficient RNA processing signals (e.g., splicing signals, polyadenylation signals), sequences that stabilize cytoplasmic mRNA, sequences that enhance translation efficiency (e.g., ribosome binding sites [e.g., Shine-Dalgamo sequences]), sequences that enhance protein stability, sequences that enhance protein secretion, and combinations thereof.

### Recombinant Vector

In another aspect, provided herein is a recombinant vector that comprises the recombinant expression construct according to any of the above or a fragment thereof (e.g., a polynucleotide that comprises a milk protein coding sequence and optional secretion signal sequence, which upon integration into the genome of a host cell creates the recombinant expression construct according to any of the above).

The recombinant vector can comprise a single recombinant expression construct according to any of the above, or two or more recombinant expression constructs according to any of the above, which can be identical or at least two of which can be not identical (e.g., differ from each other in a promoter sequence, a secretion signal, a protein coding sequence, a termination sequence, and/or an additional regulatory element). In embodiments in which the recombinant vector comprises two or more recombinant expression constructs, the two or more recombinant expression constructs can encode the same recombinant milk protein. In some such embodiments, the two or more recombinant expression constructs encoding the same recombinant milk protein differ from each other in a promoter sequence, secretion signal sequence, termination sequence, and/or additional regulatory element.

The recombinant vector can further comprise one or more other elements suitable for propagation of the recombinant vector in a recombinant host cell. Non-limiting examples of such other elements include origins of replication and selection markers. Origins of replication and selection markers are known in the art, and include bacterial and fungal origins of replication (e.g., AMA1, ANSI). Selection markers can be resistance genes (i.e., polynucleotides that encode proteins that enable host cells to detoxify an exogenously added compound [e.g., an antibiotic compound]), auxotrophic markers (i.e., polynucleotides that encode proteins that permit a host cell to synthesize an essential component (usually an amino acid) while grown in media that lacks that essential component), or color markers (i.e., genes that encode proteins that can produce a color). Non-limiting examples of suitable selection markers include amdS (acetamidase), argB (ornithine carbamoyltransferase), bar (phosphinothricin acetyltransferase), hph (hygromycin phosphotransferase), niaD (nitrate reductase), pyrG (orotidine 5′-phosphate decarboxylase), sC (sulfate adenyltransferase), trpC (anthranilate synthase), and ble (bleomycin-type antibiotic resistance), and derivatives thereof. The selection marker can comprise an alteration that decreases production of the selective marker, thus increasing the number of copies needed to permit a recombinant host cell comprising the recombinant vector to survive under selection. Selection can also be accomplished by co-transformation, wherein the transformation is carried out with a mixture of two vectors and the selection is made for one vector only.

The recombinant vector can further comprise sequences for integration by homologous (i.e., targeted integration) or nonhomologous recombination into the genome of a host cell. The recombinant expression construct can comprise at least 10, at least 25, at least 50, at least 100, at least 250, at least 500, at least 750, at least 1,000, or at least 10,000 base pairs that have sufficient identity with a target sequence in the genome of the host cell to enhance the probability of homologous recombination of the recombinant expression construct. Such homologous sequence may be non-coding or coding.

The recombinant vector according to any of the above can be isolated and/or purified.

### Method for Obtaining Recombinant Host Cell

In another aspect, provided herein is a method for obtaining the recombinant host cell according to any of the above, wherein the method comprises any combination, in any order, of the steps of: a) obtaining a polynucleotide that encodes the recombinant milk protein (and optional secretion signal) according to any of the above; b) obtaining the recombinant expression construct that encodes the recombinant milk protein (and optional secretion signal) according to any of the above, c) obtaining the recombinant vector that encodes the recombinant milk protein (and optional secretion signal) according to any of the above; and d) introducing the polynucleotide, recombinant expression construct, or recombinant vector into a host cell (e.g., any of the host cells disclosed herein) to obtain the recombinant host cell according to any of the above.

The polynucleotide, recombinant expression construct, and/or recombinant vector can be obtained by any suitable method known in the art, including, without limitation, direct chemical synthesis and cloning. The polynucleotide that encodes the recombinant milk protein according to any of the above can be obtained by genetic modification of a polynucleotide encoding a corresponding native milk protein. Such genetic modification can consist of, for example, an insertion, a substitution, a duplication, a rearrangement and/or a deletion of one or more nucleotides comprised in the polynucleotide. Such genetic modification can, for example, create a point mutation, missense mutation, substitution mutation, deletion mutation, frameshift mutation, insertion mutation, duplication mutation, amplification mutation, translocation mutation, or inversion mutation.

The recombinant host cell according to any of the above can be derived from any wild type unicellular organism, including any bacterium, fungus (e.g., yeast, filamentous fungus), archaea, unicellular protista, unicellular animal, unicellular plant, unicellular algae, protozoan, and unicellular chromista, or from a genetic variant (e.g., mutant) thereof, as well as from any generally recognized as safe (GRAS) industrial host cell.

Non-limiting examples of suitable yeast include members of any of the following genera, and derivatives and crosses thereof: Candida (e.g., Candida albicans, Candida etchellsii, Candida guillermondii, Candida humilis, Candida lipoltica, Candida orthopsilosis, Candida palmioleophila, Candida pseudotropicahs, Candida sp., Candida utilis, Candida versatilis), Cladosporium, Cryptococcus (e.g., Cryptococcus terricolus, Cryptococcus curvatus), Debaryomyces (e.g., Debaryomyces hansenii), Endomyces (e.g., Endomyces vernalis), Endomycopsis (e.g., Endomycopsis vernalis), Eremothecium (e.g., Eremothecium ashbyii), Hansenula (e.g., Hansenula sp., Hansenula polymorpha), Kluyveromyces (e.g., Kluyveromyces sp., Kluyveromyces lactis, Kluyveromyces marxianus var. lactis, Kluyveromyces marxianus, Kluyveromyces thermotolerans), Lipomyces (e.g., Lipomyces starkeyi, Lipomyces lipofer), Ogataea (e.g., Ogataea minuta), Pichia (e.g., Pichia sp., Pichia pastoris (Komagataella phaffii), Pichia finlandica, Pichia trehalophila, Pichia koclamae, Pichia membranaefaciens, Pichia minuta, Pichia lindneri), Pichia opuntiae, Pichia thermotolerans, Pichia salictaria, Pichia guercuum, Pichia pijperi, Pichia stiptis, Pichia methanolica), Rhodosporidium (e.g., Rhodosporidium toruloides), Rhodotorula (e.g., Rhodotorula sp., Rhodotorula gracilis, Rhodotorula glutinis, Rhodotorula graminis), Saccharomyces (e.g., Saccharomyces sp., Saccharomyces bayanus, Saccharomyces beticus, Saccharomyces cerevisiae, Saccharomyces chevalieri, Saccharomyces diastaticus, Saccharomyces ellipsoideus, Saccharomyces exiguus, Saccharomyces florentinus, Saccharomyces fragilis, Saccharomyces pastorianus, Saccharomyces pombe, Saccharomyces sake, Saccharomyces uvarum), Sporobolomyces (e.g., Sporobolomyces roseus), Sporidiobolus (e.g., Sporidiobolus johnsonii, Sporidiobolus salmonicolor), Trichosporon (e.g., Trichosporon cacaoliposimilis, Trichosporon oleaginosus sp. nov., Trichosporon cacaoliposimilis sp. nov., Trichosporon gracile, Trichosporon dulcitum, Trichosporon jirovecii, Trichosporon insectorum), Xanthophyllomyces (e.g., Xanthophyllomyces dendrorhous), Yarrowia (e.g., Yarrowia lipolytica), and Zygosaccharomyces (e.g., Zygosaccharomyces rouxii).

Non-limiting examples of suitable filamentous fungi include any holomorphic, teleomorphic, and anamorphic forms of fungi, including members of any of the following genera, and derivatives and crosses thereof: Acremonium (e.g., Acremonium alabamense), Aspergillus (e.g., Aspergillus aculeatus, Aspergillus awamori, Aspergillus clavatus, Aspergillus flavus, Aspergillus foetidus, Aspergillus fumigatus, Aspergillus japonicus, Aspergillus nidulans, Aspergillus niger, Aspergillus niger var. awamori, Aspergillus ochraceus, Aspergillus oryzae, Aspergillus sojae, Aspergillus terreus, as well as Emericella, Neosartorya, and Petromyces species), Aureobasidium, Canariomyces, Chaetomium, Chaetomidium, Corynascus, Chrysosporium (e.g., Chrysosporium botryoides, Chrysosporium carmichaeli, Chrysosporium crassitunicatum, Chrysosporium europae, Chrysosporium evolceannui, Chrysosporium farinicola, Chrysosporium fastidium, Chrysosporium filiforme, Chrysosporium georgiae, Chrysosporium globiferum, Chrysosporium globiferum var. articulatum, Chrysosporium globiferum var. niveum, Chrysosporium hirundo, Chrysosporium hispanicum, Chrysosporium holmii, Chrysosporium indicum, Chrysosporium iops, Chrysosporium keratinophilum, Chrysosporium kreiselii, Chrysosporium kuzurovianum, Chrysosporium lignorum, Chrysosporium obatum, Chrysosporium lucknowense, Chrysosporium lucknowense Garg 27K, Chrysosporium medium, Chrysosporium medium var. spissescens, Chrysosporium mephiticum, Chrysosporium merdarium, Chrysosporium merdarium var. roseum, Chrysosporium minor, Chrysosporium pannicola, Chrysosporium parvum, Chrysosporium parvum var. crescens, Chrysosporium pilosum, Chrysosporium pseudomerdarium, Chrysosporium pyriformis, Chrysosporium queenslandicum, Chrysosporium sigleri, Chrysosporium sulfureum, Chrysosporium synchronum, Chrysosporium tropicum, Chrysosporium undulatum, Chrysosporium vallenarense, Chrysosporium vespertilium, Chrysosporium zonatum), Coonemeria, Cunninghamella (e.g., Cunninghamella ehinulata), Dactylomyces, Emericella, Filibasidium, Fusarium (e.g., Fusarium moniliforme, Fusarium venenatum, Fusarium oxysporum, Fusarium graminearum, Fusarium proliferatum, Fusarium verticiollioides, Fusarium culmorum, Fusarium crookwellense, Fusarium poae, Fusarium sporotrichioides, Fusarium sambuccinum, Fusarium torulosum, as well as associated Gibberella teleomorphic forms thereof), Gibberella, Humicola, Hypocrea, Lentinula, Malbranchea (e.g., Malbranchea filamentosa), Magnaporthe, Malbranchium, Melanocarpus, Mortierella (e.g., Mortierella alpina 1S-4, Mortieralla isabelline, Mortierrla vinacea, Mortieralla vinaceae var. raffnoseutilizer), Mucor (e.g., Mucor miehei Cooney et Emerson (Rhizomucor miehei (Cooney & R, Emerson)) Schipper, Mucor pusillus Lindt, Mucor circinelloides Mucor mucedo), Myceliophthora (e.g., Myceliophthora thermophila), Myrothecium, Neocallimastix, Neurospora (e.g., Neurospora crassa), Paecilomyces, Penicillium (e.g., Penicillium chrysogenum, Penicillium iilacinum, Penicillium roquefortii), Phenerochaete, Phlebia, Piromyces, Pythium, Rhizopus (e.g., Rhizopus niveus), Schizophyllum, Scytalidium, Sporotrichum (e.g., Sporotrichum cellulophilum), Stereum, Talaromyces, Thermoascus, Thermomyces, Thielavia (e.g., Thielavia terrestris), Tolypocladium, and Trichoderma (e.g., Trichoderma harzianum, Trichoderma koningii, Trichoderma longibrachiatum, Trichoderma reesei, Trichoderma atroviride, Trichoderma virens, Trichoderma citrinoviride, Trichoderma viride).

Non-limiting examples of suitable bacteria include firmicutes, cyanobacteria (blue-green algae), oscillatoriophcideae, bacillales, lactobacillales, oscillatoriales, bacillaceae, lactobacillaceae, and members of any of the following genera, and derivatives and crosses thereof: Acinetobacter, Acetobacter (e.g., Acetobacter suboxydans, Acetobacter xylinum), Actinoplane (e.g., Actinoplane missouriensis), Arthrospira (e.g., Arthrospira platensis, Arthrospira maxima), Bacillus (e.g., Bacillus cereus, Bacillus coagulans, Bacillus licheniformis, Bacillus stearothermophilus, Bacillus subtilis), Escherichia (e.g., Escherichia coli), Lactobacillus (e.g., Lactobacillus acidophilus, Lactobacillus bulgaricus), Lactococcus (e.g., Lactococcus lactis, Lactococcus lactis Lancefield Group N, Lactobacillus reuteri), Leuconostoc (e.g., Leuconostoc citrovorum, Leuconostoc dextranicum, Leuconostoc mesenteroides), Micrococcus (e.g., Micrococcus lysodeikticus), Rhodococcus (e.g., Rhodococcus opacus, Rhodococcus opacus strain PD630), Spirulina, Streptococcus (e.g., Streptococcus cremoris, Streptococcus lactis, Streptococcus lactis subspecies diacetylactis, Streptococcus thermophilus), Streptomyces (e.g., Streptomyces chattanoogensis, Streptomyces griseus, Streptomyces natalensis, Streptomyces olivaceus, Streptomyces olivochromogenes, Streptomyces rubiginosus), Tetrahymena (e.g., Tetrahymena thermophile, Tetrahymena hegewischi, Tetrahymena hyperangularis, Tetrahymena malaccensis, Tetrahymena pigmentosa, Tetrahymena pyriformis, Tetrahymena vorax), and Xanthomonas (e.g., Xanthomonas campestris).

Non-limiting examples of suitable algae include members of any of the following genera, and derivatives and crosses thereof: red algae, brown algae, green algae, microalgae, Achnanthes (e.g., Achnanthes orientalis), Agmenellum, Alaria (e.g., Alaria marginata), Amphiprora (e.g., Amphiprora hyaline), Amphora (e.g., Amphora coffeiformis, Amphora coffeiformis tenuis, Amphora coffeiformis punctata, Amphora coffeiformis taylori, Amphora coffeiformis tenuis, Amphora delicatissima, Amphora delicatissima capitata, Amphora sp.), Anabaena, Analipus (e.g., Analipus japonicus), Ankistrodesmus (e.g., Ankistrodesmus falcatus), Ascophyllum (e.g., Ascophyllum nodosum), Boekelovia (e.g., Boekelovia hooglandii), Borodinella (e.g., Borodinella sp.), Botryococcus (e.g., Botryococcus braunii, Botryococcus sudeticus), Carteria, Chaetoceros (e.g., Chaetoceros gracilis, Chaetoceros muelleri, Chaetoceros muelleri subsalsum, Chaetoceros sp.), Chlorella (e.g., Chlorella anitrata, Chlorella antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolata, Chlorella glucotropha, Chlorella infusionum, Chlorella infusionism var. actophila, Chlorella infusionism var. auxenophila, Chlorella kessleri, Chlorella lobophora (strain SAG 37.88), Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella parva, Chlorella photophila, Chlorella pringsheimii, Chlorella protothecoides, Chlorella protothecoides var. acidicola, Chlorealla, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris, Chlorella vulgaris f. tertia, Chlorella vulgaris var. autotrophica, Chlorella vulgaris var. viridis, Chlorella vulgaris var. vulgaris, Chlorella vulgaris var. vulgaris f. tertia, Chlorella vulgaris var. vulgaris f. viridis, Chlorella xanthella, Chlorella zofingiensis, Chlorella trebouxioides, Chlorella vulgaris), Chlorococcum (e.g., Chlorococcum infusionum, Chlorococcum sp.), Chlorogonium, Chondrus (e.g., Chondrus crispus, Chondrus ocellatus), Chroomonas (e.g., Chroomonas sp.), Chrysosphaera (e.g., Chrysosphaera sp.), Cricosphaera (e.g., Cricosphaera sp.), Cryptomonas (e.g., Cryptomonas sp.), Cyclotella (e.g., Cyclotella cryptica, Cyclotella meneghiniana, Cyclotella sp.), Dunaliella (e.g., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta), Ecklonia (e.g., Ecklonia sp), Eisenia (e.g., Eisenia bicyclis), Ellipsoidon (e.g., Ellipsoidon sp.), Eremosphaera (e.g., Eremosphaera viridis, Eremosphaera sp.), Eucheuma (e.g., Eucheuma cottonii, Eucheuma spinosum), Euglena, Fragilaria (e.g., Fragilaria crotonensis, Fragilaria sp.), Franceia (e.g., Franceia sp.), Furcellaria (e.g., Furcellaria fastigiate), Gigartina (e.g., Gigartina acicularis, Gigartina bursa-pastoris, Gigartina pistillata, Gigartina radula, Gigartina skottsbergii, Gigartina stellate), Gleocapsa (e.g., Gleocapsa sp.), Gloeothammion (e.g., Gloeothammion sp.), Gloiopeltis (e.g., Gloiopeltis furcate), Gracilaria (e.g., Gracilaria bursa-pastoris, Gracilaria lichenoides), Hizikia (e.g., Hizikia fusiforme), Hymenomonas (e.g., Hynenomonas sp.), Isochrysis (e.g., Isochrysis aff. galbana, Isochrysis galbana), Kjellmaniella (e.g., Kjellmanaella gyrate), Laminaria (e.g., Laminaria angustata, Laminaria longirruris, Laminaria longissima, Laminaria ochotensis, Laminaria claustonia, Laminaria saccharina, Laminaria digitata, Laminaria japonica), Lepocinclis, Macrocystis (e.g., Macrocystis pyrifera), Micractinium, Monoraphidium (e.g., Monoraphidium minutum, Monoraphidium sp.), Nannochloris (e.g., Nannochloris sp.), Nannochloropsis (e.g., Nannochloropsis salina, Nannochloropsis sp.), Navicula (e.g., Navicula acceptata, Navicula biskanterae, Navicula pseudotenelloides, Navicula pelliculosa, Navicula saprophila, Navicula sp.), Nephrochloris (e.g., Nephrochloris sp.), Nephroselmis (e.g., Nephroselmis sp.), Nitzschia (e.g., Nitzschia communis, Nitzschia alexandrina, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp.), Ochromonas (e.g., Ochromonas sp.), Oocystis (e.g., Oocystis parva, Oocystis pusilla, Oocystis sp.), Oscillatoria (e.g., Oscillatoria limnetica, Oscillatoria sp., Oscillatoria subbrevis), Palmaria (e.g., Palmaria palmata), Pascheria (e.g., Pascheria acidophila), Pavlova (e.g., Pavlova sp.), Petalonia (e.g., Petalonia fascia), Phagus, Phormidium, Platymonas (e.g., Platymonas sp.), Pleurochrysis (e.g., Pleurochrysis carterae, Pleurochrysis dentate, Pleurochrysis sp.), Porphyra (e.g., Porphyra columbina, Porphyra crispata, Porphyra deutata, Porphyra perforata, Porphyra suborbiculata, Porphyra tenera), Porphyridium (e.g., Porphyridium cruentum, Porphyridium purpureum, Porphyridium aerugineum), Prototheca (e.g., Prototheca wickerhamii, Prototheca stagnora, Prototheca portoricensis, Prototheca moriformis, Prototheca zopfii), Pyramimonas (e.g., Pyramimonas sp.), Pyrobonrys, Rhodella (e.g., Rhodella maculate, Rhodella reliculata, Rhodella violacea), Rhodymnenia (e.g., Rhodymenia palmata), Sarcinoid (e.g., Sarcinoid chrysophyte), Scenedesmus (e.g., Scenedesmus armatus), Scytosiphon (e.g., Scytosiphon lome), Spirogyra, Spirulina (e.g., Spirulina platensis), Stichococcus (e.g., Stichococcus sp.), Synechococcus (e.g., Synechococcus sp.), Tetraedron, Tetraselmis (e.g., Tetraselmis sp., Tetraselmis suecica), Thalassiosira (e.g., Thalassiosira weissflogii), and Viridiella (e.g., Viridiella fridericiana).

The recombinant host cell according to any of the above can comprise genetic modifications that improve production of the recombinant milk protein. Non-limiting examples of suitable genetic modifications include altered kinase activities, altered phosphatase activities, altered protease activities, altered gene expression induction pathways, altered production and/or activity of a protein involved in protein folding, and altered production and/or activity of a protein involved in protein secretion (e.g., vesicular transport).

Methods for introducing a polynucleotide, recombinant expression construct, or recombinant vector into a host cell are well-known in the art. Non-limiting examples of such methods include calcium phosphate transfection, dendrimer transfection, liposome transfection (e.g., cationic liposome transfection), cationic polymer transfection, DEAE-dextran transfection, cell squeezing, sonoporation, optical transfection, protoplast fusion, protoplast transformation, impalefection, hydrodynamic delivery, gene gun, magnetofection, viral transduction, electroporation, and chemical transformation (e.g., using PEG).

Methods for identifying a recombinant host cell are well-known in the art, and include screening for expression of a drug resistance or auxotrophic marker encoded by the polynucleotide, recombinant expression construct, or recombinant vector that permits selection for or against growth of cells, or by other means (e.g., detection of light emitting peptide comprised in the polynucleotide, recombinant expression construct, or recombinant vector, molecular analysis of individual recombinant host cell colonies [e.g., by restriction enzyme mapping. PCR amplification. Southern analysis, or sequence analysis of isolated extrachromosomal vectors or chromosomal integration sites]).

Production of the recombinant milk protein by the recombinant host cell according to any of the above can be evaluated using any suitable method known in the art, such as assays that are carried out at the RNA level and, most suitable, at the protein level, or by use of functional bioassays that measure the production or activity of the recombinant protein. Non-limiting examples of such assays include Northern blotting, dot blotting (DNA or RNA), RT-PCR (reverse transcriptase polymerase chain reaction), RNA-Seq, in situ hybridization, Southern blotting, enzyme activity assays, immunological assays (e.g., immunohistochemical staining, immunoassays, Western blotting, ELISA), and free thiol assays (e.g., for measuring production of protein comprising free cysteine residues).

### Method for Producing Recombinant Milk Protein

In another aspect, provided herein is a method for producing the recombinant milk protein according to any of the above, wherein the method comprises the step of fermenting the recombinant host cell according to any of the above in a culture medium under conditions suitable for production of the recombinant milk protein.

The method can further comprise the steps of: purifying the recombinant milk protein from the fermentation broth to obtain a preparation comprising the recombinant milk protein; and/or post-processing the recombinant milk protein.

Alternatively, the recombinant milk protein can be obtained using in vitro methods (e.g., using cell-free transcription and/or translation systems).

### Fermenting

Suitable conditions for producing the recombinant milk protein are typically those under which the recombinant host cell according to any of the above can grow and/or remain viable, and produce the recombinant milk protein.

Non-limiting examples of suitable conditions include a suitable culture medium (e.g., a culture medium having a suitable nutrient content [e.g., a suitable carbon content, a suitable nitrogen content, a suitable phosphorus content], a suitable supplement content, a suitable trace metal content, a suitable pH), a suitable temperature, a suitable feed rate, a suitable pressure, a suitable level of oxygenation, a suitable fermentation duration (i.e., volume of culture media comprising the recombinant host cells), a suitable fermentation volume (i.e., volume of culture media comprising the recombinant host cells), and a suitable fermentation vessel.

Suitable culture media include all culture media in which the recombinant host cell can grow and/or remain viable, and produce the recombinant milk protein. Typically, the culture medium is an aqueous medium that comprises a carbon source, an assimilable nitrogen source (i.e., a nitrogen-containing compound capable of releasing nitrogen in a form suitable for metabolic utilization by the recombinant host cell), and a phosphate source.

Non-limiting examples of carbon sources include monosaccharides, disaccharides, polysaccharides, acetate, ethanol, methanol, glycerol, methane, and combinations thereof. Non-limiting examples of monosaccharides include dextrose (glucose), fructose, galactose, xylose, arabinose, and combinations thereof. Non-limiting examples of disaccharides include sucrose, lactose, maltose, trehalose, cellobiose, and combinations thereof. Non-limiting examples of polysaccharides include starch, glycogen, cellulose, amylose, hemicellulose, maltodextrin, and combinations thereof.

Non-limiting examples of assimilable nitrogen sources include anhydrous ammonia, ammonium sulfate, ammonium hydroxide, ammonium nitrate, diammonium phosphate, monoammonium phosphate, ammonium pyrophosphate, ammonium chloride, sodium nitrate, urea, peptone, protein hydrolysates, corn steep liquor, corn steep solids, spent grain, spent grain extract, and yeast extract. Use of ammonia gas is convenient for large scale operations, and can be employed by bubbling through the aqueous ferment (fermentation medium) in suitable amounts. At the same time, such ammonia can also be employed to assist in pH control.

The culture medium can further comprise an inorganic salt, a mineral (e.g., magnesium, calcium, potassium, sodium; e.g., in suitable soluble assimilable ionic and combined forms), a metal or transition metal (e.g., copper, manganese, molybdenum, zinc, iron, boron, iodine; e.g., in suitable soluble assimilable form), a vitamin, and any other nutrient or functional ingredient (e.g., a protease [e.g., a plant-based protease] that can prevent degradation of the recombinant milk protein, a protease inhibitor that can reduce the activity of a protease that can degrade the recombinant milk protein, and/or a sacrificial protein that can siphon away protease activity, an anti-foaming agent, an anti-microbial agent, a surfactant, an emulsifying oil).

Suitable culture media are available from commercial suppliers or may be prepared according to published compositions (e.g., in catalogues of the American Type Culture Collection).

A suitable pH can be a pH of between 2 and 8, 7.5, 7, 6.5, 6, 5.5, 5.4, 5.3, 5.2, 5.1, 5, 4.9, 4.8, 4.7, 4.6, 4.5, 4, 3.5, 3, or 2.5; between 2.5 and 8, 7.5, 7, 6.5, 6, 5.5, 5.4, 5.3, 5.2, 5.1, 5, 4.9, 4.8, 4.7, 4.6, 4.5, 4, 3.5, or 3; between 3 and 8, 7.5, 7, 6.5, 6, 5.5, 5.4, 5.3, 5.2, 5.1, 5, 4.9, 4.8, 4.7, 4.6, 4.5, 4, or 3.5; between 3.5 and 8, 7.5, 7, 6.5, 6, 5.5, 5.4, 5.3, 5.2, 5.1, 5, 4.9, 4.8, 4.7, 4.6, 4.5, or 4; between 4 and 8, 7.5, 7, 6.5, 6, 5.5, 5.4, 5.3, 5.2, 5.1, 5, 4.9, 4.8, 4.7, 4.6, or 4.5; between 4.5 and 8, 7.5, 7, 6.5, 6, 5.5, 5.4, 5.3, 5.2, 5.1, 5, 4.9, 4.8, 4.7, or 4.6; between 4.6 and 8, 7.5, 7, 6.5, 6, 5.5, 5.4, 5.3, 5.2, 5.1, 5, 4.9, 4.8, or 4.7; between 4.7 and 8, 7.5, 7, 6.5, 6, 5.5, 5.4, 5.3, 5.2, 5.1, 5, 4.9, or 4.8; between 4.8 and 8, 7.5, 7, 6.5, 6, 5.5, 5.4, 5.3, 5.2, 5.1, 5, or 4.9; between 4.9 and 8, 7.5, 7, 6.5, 6, 5.5, 5.4, 5.3, 5.2, 5.1, or 5; between 5 and 8, 7.5, 7, 6.5, 6, 5.5, 5.4, 5.3, 5.2, or 5.1; between 5.1 and 8, 7.5, 7, 6.5, 6, 5.5, 5.4, 5.3, or 5.2; between 5.2 and 8, 7.5, 7, 6.5, 6, 5.5, 5.4, or 5.3; between 5.3 and 8, 7.5, 7, 6.5, 6, 5.5, or 5.4; between 5.4 and 8, 7.5, 7, 6.5, 6, or 5.5; between 5.5 and 8, 7.5, 7, 6.5, or 6; between 6 and 8, 7.5, 7, or 6.5; between 6.5 and 8, 7.5, or 7; between 7 and 8, or 7.5; or between 7.5 and 8.

A suitable temperature can be a temperature of between 20° C., and 46° C., 44° C., 42° C., 40° C. 38° C., 36° C., 34° C., 32° C. 30° C., 28° C., 26° C., 24° C. or 22° C.; between 22° C., and 46° C., 44° C., 42° C., 40° C., 38° C., 36° C., 34° C., 32° C., 30° C., 28° C. 26° C., or 24° C.; between 24° C., and 46° C., 44° C., 42° C., 40° C., 38° C., 36° C., 34° C., 32° C., 30° C., 28° C., or 26° C.; between 26° C., and 46° C., 44° C., 42° C., 40° C., 38° C., 36° C., 34° C., 32° C., 30° C., or 28° C.; between 28° C., and 46° C., 44° C., 42° C., 40° C., 38° C., 36° C., 34° C., 32° C., or 30° C.; between 30° C., and 46° C., 44° C., 42° C., 40° C., 38° C., 36° C., 34° C., or 32° C.; between 32° C., and 46° C., 44° C., 42° C., 40° C., 38° C., 36° C., or 34° C.; between 36° C., and 46° C., 44° C., 42° C., 40° C., or 38° C.; between 38° C., and 46° C., 44° C., 42° C., or 40° C.; between 40° C., and 46° C., 44° C., or 42° C. between 42° C., and 460C or 44° C.; or between 44° C., and 46° C.

A suitable feed rate can be a feed rate of between 0.01 g and 0.2 g glucose equivalent per g dry cell weight (DCW) per hour.

A suitable pressure can be a pressure of between 0 psig and 50 psig, 40 psig, 30 psig, 20 psig, or 10 psig; between 10 psig and 50 psig, 40 psig, 30 psig, or 20 psig: between 20 psig and 50 psig, 40 psig, or 30 psig; between 30 psig and 50 psig, or 40 psig; or between 40 psig and 50 psig.

A suitable oxygenation can be an aeration rate of between 0.1 volumes of oxygen per liquid volume in the fermentor per minute (vvm) and 2.1 vvm, 1.9 vvm, 1.7 vvm, 1.5 vvm, 1.3 vvm, 1.1 vvm, 0.9 vvm, 0.7 vvm, 0.5 vvm, or 0.3 vvm; between 0.3 vvm and 2.1 vvm, 1.9 vvm, 1.7 vvm, 1.5 vvm, 1.3 vvm, 1.1 vvm, 0.9 vvm, 0.7 vvm, or 0.5 vvm; between 0.5 vvm and 2.1 vvm, 1.9 vvm, 1.7 vvm, 1.5 vvm, 1.3 vvm, 1.1 vvm, 0.9 vvm, or 0.7 vvm; between 0.7 vvm and 2.1 vvm, 1.9 vvm, 1.7 vvm, 1.5 vvm, 1.3 vvm, 1.1 vvm, or 0.9 vvm; between 0.9 vvm and 2.1 vvm, 1.9 vvm, 1.7 vvm, 1.5 vvm, 1.3 vvm, or 1.1 vvm; between 1.1 vvm and 2.1 vvm, 1.9 vvm, 1.7 vvm, 1.5 vvm, or 1.3 vvm; between 1.3 vvm and 2.1 vvm, 1.9 vvm, 1.7 vvm, or 1.5 vvm; between 1.5 vvm and 2.1 vvm, 1.9 vvm, or 1.7 vvm; between 1.7 vvm and 2.1 vvm or 1.9 vvm; or between 1.9 vvm and 2.1 vvm.

A suitable fermentation duration can be a fermentation duration of between 10 hours and 500 hours, 400 hours, 300 hours, 200 hours, 100 hours, 50 hours, 40 hours, 30 hours, or 20 hours; between 20 hours and 500 hours, 400 hours, 300 hours, 200 hours, 100 hours, 50 hours, 40 hours, or 30 hours; between 30 hours and 500 hours, 400 hours, 300 hours, 200 hours, 100 hours, 50 hours, or 40 hours; between 40 hours and 500 hours, 400 hours, 300 hours, 200 hours, 100 hours, or 50 hours, between 50 hours and 500 hours, 400 hours, 300 hours, 200 hours, or 100 hours; between 100 hours and 500 hours, 400 hours, 300 hours, or 200 hours; between 200 hours and 500 hours, 400 hours, or 300 hours; between 300 hours and 500 hours, or 400 hours; or between 400 hours and 500 hours.

A suitable fermentation volume can be between 1 L and 10,000,000 L, 5,000,000 L, 1,000,000 L, 500,000 L, 100,000 L, 50,000 L, 10,000 L, 5,000 L, 1,000 L, 500 L, 100 L, 50 L, or 10 L; between 10 L and 10,000,000 L, 5,000,000 L, 1,000,000 L, 500,000 L, 100,000 L, 50,000 L, 10, 000 L, 5,000 L, 1,000 L, 500 L, 100 L, or 50 L; between 50 L and 10,000,000 L, 5,000,000 L, 1,000,000 L, 500,000 L, 100,000 L, 50,000 L, 10, 000 L, 5,000 L, 1,000 L, 500 L, or 100 L; between 100 L and 10,000,000 L, 5,000,000 L, 1,000,000 L, 500,000 L, 100,000 L, 50,000 L, 10, 000 L, 5,000 L, 1,000 L, or 500 L; between 500 L and 10,000,000 L, 5,000,000 L, 1,000,000 L, 500,000 L, 100,000 L, 50,000 L, 10, 000 L, 5,000 L, or 1,000 L; between 1,000 L and 10,000,000 L, 5,000,000 L, 1,000,000 L, 500,000 L, 100,000 L, 50,000 L, 10, 000 L, or 5,000 L; between 5,000 L and 10,000,000 L, 5,000,000 L, 1,000,000 L, 500,000 L, 100,000 L, 50,000 L, or 10, 000 L; between 10,000 L and 10,000,000 L, 5,000,000 L, 1,000,000 L, 500,000 L, 100,000 L, or 50,000 L; between 50,000 L and 10,000,000 L, 5,000,000 L, 1,000,000 L, 500,000 L, or 100,000 L; between 100,000 L and 10,000,000 L, 5,000,000 L, 1,000,000 L, or 500,000 L; between 500,000 L and 10,000,000 L, 5,000,000 L, or 1,000,000 L; between 1,000,000 L and 10,000,000 L, or 5,000,000 L, or between 5,000,000 L and 10,000,000 L.

A suitable fermentation vessel can be any fermentation vessel known in the art. Non-limiting examples of suitable fermentation vessels include culture plates, shake flasks, fermentors (e.g., stirred tank fermentors, airlift fermentors, bubble column fermentors, fixed bed bioreactors, laboratory fermentors, industrial fermentors, or any combination thereof), used at any suitable scale (e.g., small-scale, large-scale) and in any process (e.g., solid culture, submerged culture, batch, fed-batch, or continuous-flow).

### Purifying

Methods for purifying a recombinant protein (e.g., from a fermentation broth) to obtain a preparation comprising the recombinant protein are well-known in the art (see, for example, Protein Purification, J C Janson and L Ryden, Eds., VCH Publishers, New York, 1989; Protein Purification Methods: A Practical Approach, ELV Harris and S Angel, Eds., IRL Press, Oxford, England, 1989, respectively), and can be adapted to purify the recombinant milk protein according to any of the above.

The recombinant milk protein according to any of the above can be purified on the basis of its molecular weight, for example, by size exclusion/exchange chromatography, ultrafiltration through membranes, gel permeation chromatography (e.g., preparative disc-gel electrophoresis), or density centrifugation.

The recombinant milk protein according to any of the above also can be purified on the basis of its surface charge or hydrophobicity/hydrophilicity, for example, by isoelectric precipitation, anion/cation exchange chromatography, isoelectric focusing (IEF), or reverse phase chromatography.

The recombinant milk protein according to any of the above also can be purified on the basis of its solubility, for example, by ammonium sulfate precipitation, isoelectric precipitation, surfactants, detergents, or solvent extraction.

The recombinant milk protein according to any of the above also can be purified on the basis of its affinity to another molecule, for example, by affinity chromatography, reactive dyes, or hydroxyapatite. Affinity chromatography can include the use of an antibody having a specific binding affinity for the recombinant milk protein, or a lectin to bind to a sugar moiety on the recombinant milk protein, or any other molecule that specifically binds the recombinant milk protein. The recombinant milk protein can comprise a tag peptide or polypeptide operably linked to its C- or N-terminus to facilitate affinity-based purification of the recombinant milk protein. Non-limiting examples of suitable tag peptides or polypeptides include affinity tags (i.e., peptides or polypeptides that bind to certain agents or matrices), solubilization tags (i.e., peptides or polypeptides that assist in proper folding of proteins and prevent precipitation), chromatography tags (i.e., peptides or polypeptides that alter the chromatographic properties of a protein to afford different resolution across a particular separation techniques), epitope tags (i.e., peptides or polypeptides that are bound by antibodies), fluorescence tags, chromogenic tags, enzyme substrate tags (i.e., peptides or polypeptides that are the substrates for specific enzymatic reactions), chemical substrate tags (i.e., peptides or polypeptides that are the substrates for specific chemical modifications), self-cleaving tags (peptides or polypeptides that possess inducible proteolytic activity; e.g., Sortase tag, Npro tag, FrpC module, CPD), hydrophobic tags (proteins or polypeptides that are highly hydrophobic and direct the protein for inclusion body formation; e.g., KSI tag, TrpE tag), or combinations thereof. Non-limiting examples of suitable affinity tags include maltose binding protein (MBP) tag, glutathione-S-transferase (GST) tag, poly(His) tag, SBP-tag. Strep-tag, and calmodulin-tag. Non-limiting examples of suitable solubility tags include thioredoxin (TRX) tag, poly(NANP) tag, MBP tag, SUMO tag, GB1 tag, NUSA CBD tag, and GST tag. Non-limiting examples of chromatography tags include polyanionic amino acid tags (e.g., FLAG-tag) and polyglutamate tag. Non-limiting examples of epitope tags include V5-tag, VSV-tag, E-tag, NE-tag, hemagglutinin (Ha)-tag, Myc-tag, and FLAG-tag. Non-limiting examples of fluorescence tags include green fluorescent protein (GFP) tag, blue fluorescent protein (BFP) tag, cyan fluorescent protein (CFP) tag, yellow fluorescent protein (YFP) tag, orange fluorescent protein (OFP) tag, red fluorescent protein (RFP) tag, and derivatives thereof. Non-limiting examples of enzyme substrate tags include peptides or polypeptides comprising a lysine within a sequence suitable for biotinilation (e.g., AviTag, Biotin Carboxyl Carrier Protein [BCCP]). Non-limiting examples of chemical substrate tags include substrates suitable for reaction with FIAsH-EDT2. The tag peptide or polypeptide can be removed following isolation of the recombinant milk protein (e.g., via protease cleavage).

In embodiments in which the recombinant milk protein according to any of the above is secreted by the recombinant host cell according to any of the above, the recombinant milk protein can be purified directly from the culture medium. In other embodiments, the recombinant milk protein can be purified from a cell lysate.

The recombinant milk protein can be purified to a purity of greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, greater than 97%, or greater than 99% relative to other components comprised in the fermentation broth or preparation, or to at least 2-fold, at least 3-fold, at least 4-fold, at least 5-fold, at least 6-fold, at least 7-fold, at least 8-fold, at least 9-fold, or at least 10-fold greater abundancy relative to other components comprised in the fermentation broth, or to a purity of greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, greater than 97%, or greater than 99% by weight.

The identity of the recombinant milk protein can be confirmed and/or quantified by high performance liquid chromatography (HPLC), Western blot analysis, Eastern blot analysis, polyacrylamide gel electrophoresis, capillary electrophoresis, formation of an enzyme product, disappearance of an enzyme substrate, and 2-dimensional mass spectroscopy (2D-MS/MS) sequence identification.

### Post-Processing

Post-processing may alter certain chemical and/or physical properties of the recombinant milk protein, including but not limited to size, charge, hydrophobicity, hydrophilicity, solvation, protein folding, and chemical reactivity.

Post-processing can comprise refolding of the recombinant protein (e.g., by removing a denaturant), fragmenting (e.g., by chemical means or by exposure to proteases [e.g., trypsin, pepsin]), heating (e.g., to remove protein aggregates), removing reactive sites (e.g., removing reactive sites of methionine and/or tryptophan residues by oxidation), modulating (e.g., via chemical, photochemical, and/or enzymatic strategies), demineralizing (by, e.g., dialysis, ultrafiltration, reverse osmosis, ion exchange chromatography), cyclizing, removing tags and/or fusion polypeptides (e.g., by exposure to site-specific proteases), biotinylating (i.e., attaching biotin), and conjugation to other elements (e.g., poly-ethylene-glycol, antibodies, liposomes, phospholipids, DNA. RNA, polynucleotides, sugars, disaccharides, polysaccharides, starches, cellulose, detergents, cell walls).

Post-processing can occur in a random manner or in a site-specific manner (e.g., at sulfhydryl groups of cysteine residues [e.g., for aminoethylation, formation of iodoacetamides, formation of maleimides, formation of Dha, covalent attachment via disulfide bonds, and desulfurization], at primary amine groups of lysine residues [e.g., for attachment of activated esters, sulfonyl chlorides, isothiocyanates, unsaturated aldehyde esters, and aldehydes], at phenolic hydroxyl groups of tyrosine residues, at specific allergenic epitopes [e.g., glycan groups]).

The recombinant milk protein can be spray dried or concentrated via evaporation (e.g., to obtain a powder).

### Composition Comprising Recombinant Milk Protein

In another aspect, provided herein is a composition that comprises a milk protein component, wherein the milk protein component comprises or consist of the recombinant milk protein according to any of the above, and wherein the composition has an attenuated or essentially eliminated allergenicity compared to a corresponding composition.

The composition can comprise between 0.1% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, or 0.2%; between 0.2% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, or 0.3%; between 0.3% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, or 0.4%; between 0.4% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, or 0.5%; between 0.5% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, or 0.6%; between 0.6% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, or 0.7%; between 0.7% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, or 0.8%; between 0.8% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, or 0.9%; between 0.9% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, or 1%; between 1% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, or 2%; between 2% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, or 3%; between 3% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, or 4%; between 4% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, or 5%; between 5% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, or 6%; between 6% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, or 7%; between 7% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, or 8%; between 8% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, or 9%; between 9% and 100%, 95% 90%, 85%, 80%, 75%, 70%, 65%, 60%, 55%, 50%, 45%, 40%, 35%, 30%, 25%, 20%, 15%, 14%, 13%, 12%, 11%, or 10%; between 10% and 100%, 95%, 90%, 85%, 80%, 75%, 70%, 65%, 60%, 55%, 50%, 45%, 40%, 35%, 30%, 25%, 20%, 15%, 14%, 13%, 12%, or 11%; between 11% and 100%, 95%, 90%, 85%, 80%, 75%, 70%, 65%, 60%, 55%, 50%, 45%, 40%, 35%, 30%, 25%, 20%, 15%, 14%, 13%, or 12%; between 12% and 100%, 95%, 90%, 85%, 80%, 75%, 70%, 65%, 60%, 55%, 50%, 45%, 40%, 35%, 30%, 25%, 20%, 15%, 14%, or 13%; between 13% and 100%, 95%, 90%, 85%, 80%, 75%, 70%, 65%, 60%, 55%, 50%, 45%, 40%, 35%, 30%, 25%, 20%, 15%, or 14%; between 14% and 100%, 95%, 90%, 85%, 80%, 75%, 70%, 65%, 60%, 55%, 50%, 45%, 40%, 35%, 30%, 25%, 20%, or 15%; between 15% and 100%, 95%, 90%, 85%, 80%, 75%, 70%, 65%, 60%, 55%, 50%, 45%, 40%, 35%, 30%, 25%, or 20%; between 20% and 100%, 95%, 90%, 85%, 80%, 75%, 70%, 65%, 60%, 55%, 50%, 45%, 40%, 35%, 30%, or 25%; between 25% and 100%, 95%, 90%, 85%, 80%, 75%, 70%, 65%, 60%, 55%, 50%, 45%, 40%, 35%, or 30%; between 30% and 100%, 95%, 90%, 85%, 80%, 75%, 70%, 65%, 60%, 55%, 50%, 45%, 40%, or 35%; between 35% and 100%, 95%, 90%, 85%, 80%, 75%, 70%, 65%, 60%, 55%, 50%, 45%, or 40%; between 40% and 100%, 95%, 90%, 85%, 80%, 75%, 70%, 65%, 60%, 55%, 50%, or 45%; between 45% and 100%, 95%, 90%, 85%, 80%, 75%, 70%, 65%, 60%, 55%, or 50%; between 50% and 100%, 95%, 90%, 85%, 80%, 75%, 70%, 65%, 60%, or 55%; between 55% and 100%, 95%, 90%, 85%, 80%, 75%, 70%, 65%, or 60%; between 60% and 100%, 95%, 90%, 85%, 80%, 75%, 70%, or 65%; between 65% and 100%, 95%, 90%, 85%, 80%, 75%, or 70%; between 70% and 100%, 95%, 90%, 85%, 80%, or 75%; between 75% and 100%, 95%, 90%, 85%, or 80%; between 80% and 100%, 95%, 90%, or 85%; or between 85% and 100%, 95%, 90%; between 90% and 100% or 95%, or between 95% and 100% by mass of the milk protein component.

At standard ambient temperature and conditions (i.e., 20-30° C., and 0.95-1.05 atm), the composition according to any of the above can be a fluid, semi-solid (e.g., gelatinous), solid, or powder. The powder can comprise a moisture content of less than 20%, less than 15%, less than 10%, less than 7%, less than 5%, less than 3%, or less than 1%; or between 0.1% and 20%, 15%, 10%, 5%, or 1%; between 1% and 20%, 15%, 10%, or 5%; between 5% and 20%, 15%, or 10%; between 10% and 20%, or 15%; or between 15% and 20%. The powder can be used in powder form, or the powder can be reconstituted with a hydrating agent prior to use, or the powder can be mixed with other dry components (e.g., flour, sugar, minerals, pH or ionic strength adjusting agents) before a hydrating agent is added to the mixture.

The composition according to any of the above can further comprise an other ingredient (e.g., any of the other ingredients disclosed herein). For example, the composition can comprise between 0.001% and 50%, 25%, 20%, 15%, 10%, 9%, 8%, 7%, 6%, 5%, 4%, 3%, 2%, 1%, 0.5%, 0.1%, or 0.01%; between 0.01% and 50%, 25%, 20%, 15%, 10%, 9%, 8%, 7%, 6%, 5%, 4%, 3%, 2%, 1%, 0.5%, or 0.1%; between 0.1% and 50%, 25%, 20%, 15%, 10%, 9%, 8%, 7%, 6%, 5%, 4%, 3%, 2%, 1%, or 0.5%; between 0.5% and 50%, 25%, 20%, 15%, 10%, 9%, 8%, 7%, 6%, 5%, 4%, 3%, 2%, or 1%; between 1% and 50%, 25%, 20%, 15%, 10%, 9%, 8%, 7%, 6%, 5%, 4%, 3%, or 2%; between 2% and 50%, 25%, 20%, 15%, 10%, 9%, 8%, 7%, 6%, 5%, 4%, or 3%; between 3% and 50%, 25%, 20%, 15%, 10%, 9%, 8%, 7%, 6%, 5%, or 4%; between 4% and 50%, 25%, 20%, 15%, 10%, 9%, 8%, 7%, 6%, or 5%; between 5% and 50%, 25%, 20%, 15%, 10%, 9%, 8%, 7%, or 6%; between 6% and 50%, 25%, 20%, 15%, 10%, 9%, 8%, or 7%; between 7% and 50%, 25%, 20%, 15%, 10%, 9%, or 8%; between 8% and 50%, 25%, 20%, 15%, 10%, or 9%; between 9% and 50%, 25%, 20%, 15%, or 10%; between 10% and 50%, 25%, 20%, or 15%; between 15% and 50%, 25%, or 20%; between 20% and 50% or 25%; or between 25% and 50% by mass of any one such other ingredient, any combination of two or more such other ingredients, or all such other ingredients.

The composition according to any of the above can be essentially free of a component found in a mammal-produced milk (e.g., Bos taurus milk, goat milk, sheep milk, human milk), or can comprise a lower concentration of at least one component found in a mammal-produced milk. Non-limiting examples of components found in mammal-derived milk include lactose, saturated fat, cholesterol, native milk proteins, and native milk lipids.

The composition according to any of the above can be essentially free of a component obtained from an animal (i.e., a component that is native to an animal, including animal products [i.e., parts of an animal that are consumables or typically prepared for consumption by humans; e.g., animal meat, animal fat, animal blood], animal byproducts [i.e., products that are typically not consumable by themselves but are the byproducts of slaughtering animals for consumption; e.g., animal bones, animal carcasses, and constituents isolated therefrom], products produced by an animal [e.g., mammal-derived milk, chicken eggs, bee honey], and consumables produced therefrom [e.g., gelatin, rennet, whey proteins extracted from mammal-derived milk, casein extracted from mammal-derived milk, milk lipid extracted from mammal-derived milk, animal lipids, animal proteins]), or comprise 2% or less by mass of such component.

The composition according to any of the above can have an allergenicity that is lower than that of a comparable composition, such as, for example, an allergenicity of no more than 0%, 1%, 5%, 10%, 15%, 20%, 25%, 30%, 35%, 40%, 45%, 50%, 55%, 60%, 65%, 70%, 75%, 80%, 85%, 90%, or 95% relative to that of a comparable composition. Allergenicity may be measured using a skin prick test, blood test, or oral challenge.

The composition according to any of the above can be a variety of products, including, for example, food products (i.e., products that are ingested for dietary purposes), cosmetic and personal care products (e.g., ointments, lotions, creams [e.g., moisturizing creams], cleansers, massage creams, soaps, hair shampoos, hair conditioners, skin masks, finishing products, hair tonics, toothpastes, chewing gums, gum-cleaning agents, skin lotions/creams), pharmaceutical products (e.g., products used for delivery of medicinal agents [e.g., micro- or nano-particles (e.g., beads, micelles) that encapsulate a therapeutic or nutraceutical for delivery (e.g., controlled delivery)], coatings of tablets, capsules, compacts, hydrogels), polymers (i.e., molecules composed of repeated molecular units that are covalently linked, either directly with each other or via intermediary molecules), and compositions with industrial utility (e.g., dielectrics).

### Milk Protein Component

The milk protein component comprised in the composition according to any of the above consists of the recombinant milk protein according to any of the above and optionally an other milk protein.

The recombinant milk protein can be a single recombinant milk protein according to any of the above, or can be a mixture of two or more recombinant milk proteins according to any of the above.

The optional other milk protein can be a native milk protein or a recombinant milk protein not provided herein. The native or recombinant milk protein can be obtained from any mammalian species (e.g., any of the mammalian species disclosed herein). Methods for extracting native milk proteins and/or producing recombinant milk proteins are disclosed in U.S. Pat. No. 9,924,728, issued Mar. 27, 2018; U.S. publication US20190216106, published Jul. 18, 2019; and PCT publication WO2019213155, published Nov. 7, 2019; which are hereby incorporated herein in their entireties.

The optional other milk protein can be a single other milk protein, or can be two or more other milk proteins. Non-limiting examples of other milk proteins include α-lactalbumin, β-lactoglobulin, lactoferrin, transferrin, serum albumin, lactoperoxidase, glycomacropeptide (GMP), κ-casein, β-casein, γ-casein, α-S1-casein, and α-S2-casein, Without wishing to be limited by theory, it is believed that complexation of the recombinant milk protein according to any of the above with GMP can further attenuated allergenicity of the composition compared to an identical composition lacking GMP.

The milk protein component can, for example, comprise or consist of; the recombinant β-lactoglobulin according to any of the above and a native β-lactoglobulin or recombinant β-lactoglobulin not provided herein; the recombinant β-lactoglobulin according to any of the above and a native or recombinant α-lactalbumin (e.g., the recombinant α-lactalbumin according to any of the above); the recombinant β-lactoglobulin according to any of the above and a native or recombinant GMP; the recombinant β-lactoglobulin according to any of the above and a native or recombinant κ-casein; the recombinant β-lactoglobulin according to any of the above and a native or recombinant D-casein; the recombinant β-lactoglobulin according to any of the above and a native or recombinant γ-casein; the recombinant β-lactoglobulin according to any of the above and a native or recombinant α-S1-casein; the recombinant β-lactoglobulin according to any of the above and a native or recombinant α-S2-casein; the recombinant β-lactoglobulin according to any of the above, a native or recombinant α-lactalbumin (e.g., the recombinant α-lactalbumin according to any of the above), and a native or recombinant GMP; the recombinant β-lactoglobulin according to any of the above, a native or recombinant α-lactalbumin (e.g., the recombinant α-lactalbumin according to any of the above), and a native or recombinant κ-casein; the recombinant β-lactoglobulin according to any of the above, a native or recombinant α-lactalbumin (e.g., the recombinant α-lactalbumin according to any of the above), and a native or recombinant β-casein; the recombinant β-lactoglobulin according to any of the above, a native or recombinant α-lactalbumin (e.g., the recombinant α-lactalbumin according to any of the above), and a native or recombinant γ-casein; the recombinant β-lactoglobulin according to any of the above, a native or recombinant α-lactalbumin (e.g., the recombinant α-lactalbumin according to any of the above), and a native or recombinant α-S1-casein; the recombinant β-lactoglobulin according to any of the above, a native or recombinant α-lactalbumin (e.g., the recombinant α-lactalbumin according to any of the above), and a native or recombinant α-S2-casein; the recombinant β-lactoglobulin according to any of the above, a native or recombinant GMP, and a native or recombinant κ-casein; the recombinant β-lactoglobulin according to any of the above, a native or recombinant GMP, and a native or recombinant β-casein; the recombinant β-lactoglobulin according to any of the above, a native or recombinant GMP, and a native or recombinant γ-casein; the recombinant β-lactoglobulin according to any of the above, a native or recombinant GMP, and a native or recombinant α-S1-casein; the recombinant β-lactoglobulin according to any of the above, a native or recombinant GMP, and a native or recombinant α-S2-casein; the recombinant β-lactoglobulin according to any of the above, a native or recombinant κ-casein, and a native or recombinant β-casein; the recombinant β-lactoglobulin according to any of the above, a native or recombinant κ-casein, and a native or recombinant γ-casein; the recombinant β-lactoglobulin according to any of the above, a native or recombinant κ-casein, and a native or recombinant α-S1-casein; the recombinant β-lactoglobulin according to any of the above, a native or recombinant κ-casein, and a native or recombinant α-S2-casein; the recombinant β-lactoglobulin according to any of the above, a native or recombinant β-casein, and a native or recombinant γ-casein; the recombinant β-lactoglobulin according to any of the above, a native or recombinant β-casein, and a native or recombinant α-S1-casein; the recombinant β-lactoglobulin according to any of the above, a native or recombinant β-casein, and a native or recombinant α-S2-casein; the recombinant β-lactoglobulin according to any of the above, a native or recombinant GMP, a native or recombinant κ-casein, and a native or recombinant β-casein; the recombinant β-lactoglobulin according to any of the above, a native or recombinant GMP, a native or recombinant κ-casein, and a native or recombinant γ-casein; or the recombinant β-lactoglobulin according to any of the above, a native or recombinant GMP, a native or recombinant β-casein, and a native or recombinant γ-casein; the recombinant β-lactoglobulin according to any of the above, a native or recombinant GMP, a native or recombinant κ-casein, and a native or recombinant γ-casein; the recombinant β-lactoglobulin according to any of the above, a native or recombinant GMP, a native or recombinant β-casein, and a native or recombinant α-S1-casein; or the recombinant β-lactoglobulin according to any of the above, a native or recombinant GMP, a native or recombinant β-casein, and a native or recombinant α-S2-casein.

The milk protein component can consist of whey protein and casein at a mass ratio of between about 10 to 1 and about 1 to 10 (e.g., about 10 to 1, about 9 to 1, about 8 to 1, about 7 to 1, about 6 to 1, about 5 to 1, about 4 to 1, about 3 to 1, about 2 to 1, about 1 to 1, about 1 to 2, about 1 to 3, about 1 to 4, about 1 to 5, about 1 to 6, about 1 to 7, about 1 to 8, about 1 to 9, about 1 to 10).

The milk protein component can consist of recombinant milk protein and native milk protein at a mass ratio of between about 100 to 1 and about 1 to 100 (e.g., about 100 to 1, about 90 to 1, about 80 to 1, about 70 to 1, about 60 to 1, about 50 to 1, about 40 to 1, about 30 to 1, about 20 to 1, about 10 to 1, about 9 to 1, about 8 to 1, about 7 to 1, about 6 to 1, about 5 to 1, about 4 to 1, about 3 to 1, about 2 to 1, about 1 to 1, about 1 to 2, about 1 to 3, about 1 to 4, about 1 to 5, about 1 to 6, about 1 to 7, about 1 to 8, about 1 to 9, about 1 to 10, about 1 to 20, about 1 to 30, about 1 to 40, about 1 to 50, about 1 to 60, about 1 to 70, about 1 to 80, about 1 to 90, or about 1 to 100).

The milk protein component can consist of recombinant whey protein and native whey protein at a mass ratio of between about 100 to 1 and about 1 to 100 (e.g., about 100 to 1, about 90 to 1, about 80 to 1, about 70 to 1, about 60 to 1, about 50 to 1, about 40 to 1, about 30 to 1, about 20 to 1, about 10 to 1, about 9 to 1, about 8 to 1, about 7 to 1, about 6 to 1, about 5 to 1, about 4 to 1, about 3 to 1, about 2 to 1, about 1 to 1, about 1 to 2, about 1 to 3, about 1 to 4, about 1 to 5, about 1 to 6, about 1 to 7, about 1 to 8, about 1 to 9, about 1 to 10, about 1 to 20, about 1 to 30, about 1 to 40, about 1 to 50, about 1 to 60, about 1 to 70, about 1 to 80, about 1 to 90, or about 1 to 100).

### Other Ingredients

The other ingredient optionally comprised in the composition according to any of the above can be any other ingredient.

Non-limiting examples of suitable other ingredients include non-milk proteins, bioactive agents, nutritional agents, and functional agents.

The optional non-milk proteins can consist of one or more native and/or recombinant non-milk proteins derived from any source, as well as mixtures of native and/or recombinant non-milk proteins derived from various sources. Non-limiting examples of suitable sources include animals, plants, algae, fungi, or bacteria. Non-limiting examples of animal proteins include structural animal proteins (e.g., collagen, tropoelastin, elastin), egg proteins (e.g., hereinovomucoid, ovalbumin, ovotransferrin, G162M F167A ovomucoid, ovoglobulin G2, ovoglobulin G3, α-ovomucin, O-ovomucin, lysozyme, ovoinhibitor, ovoglycoprotein, flavoprotein, ovomacroglobulin, ovostatin, cystatin, avidin, ovalbumin related protein X, ovalbumin related protein Y), and globular proteins (e.g., albumin). Non-limiting examples of plant proteins include pea proteins (e.g., legumin, vicillin, covicillin) and potato proteins (e.g., tuberin, protease inhibitor notate II). The optional non-milk proteins can comprise a recombinant non-milk protein (e.g., a recombinant non-milk protein having a mammalian PTM, a non-mammalian PTM, or a mixture thereof, and/or lacking a mammalian PTM, and/or lacking an epitope that can elicit an immune response in a human or animal).

Non-limiting examples of bioactive agents include neutraceuticals (i.e., compounds that have physiological benefit or provide protection against disease), and therapeutics (i.e., compounds that treat disease).

Non-limiting examples of nutritional agents include nutritional supplements, prebiotics, probiotics, pro-vitamins, vitamins, minerals, antioxidants, carbohydrates, lipids, and essential and semi-essential amino acids.

Non-limiting examples of vitamins include lipid soluble vitamins, water soluble vitamins, thiamin (vitamin B1), riboflavin (vitamin B2), niacin (vitamin B3), pantothenic acid (vitamin B5, respectively), vitamin B6 (pyridoxine), vitamin B12 (cobalamin), vitamin C, folate, vitamins A, vitamin D, vitamin E, vitamin K, and derivatives and mixtures thereof.

Non-limiting examples of minerals include calcium, phosphorous, potassium, sodium, citrate, chloride, phosphate, sulfate, magnesium, potassium, zinc, iron, molybdenum, manganese, copper, and mixtures thereof.

Non-limiting examples of antioxidants include α-tocopherol (e.g., tocopherol comprised in Bos taurus milk), low molecular weight thiols (e.g., low molecular weight thiols comprised in Bos taurus milk), retinol (e.g., retinol comprised in Bos taurus milk), carotenoids (e.g., carotenoids comprised in cow milk, α-carotene, 0-carotene, γ-carotene, lutein, zeaxanthin, astaxanthin), vitamin E, Azadirachta indica extract, riboflavin, rosemary extract, phenolic diterpenes (e.g., carnosol, carnosic acid) comprised in rosemary extract, sage extract, ascorbic acid (vitamin C) and its salts, lactic acid and its salts, grape residue silage, phenolic compounds (e.g., ferulic acid) comprised in grape residue silage, soybean (Glycine max) extract, isoflavones or polyphenolic compounds comprised in soybean extract, garlic (Allium sativum) extract, phenolic or flavonoid, or terpenoid compounds comprised in garlic extract, fennel (Foeniculum vulgare Mill.) extract, chamomile (Matricaria recutita L.) extract, fatty acids (e.g., alpha-lipoic acid), brown algae (e.g., Ascophyllum nodosum, Fucus vesiculosus), essential oils of green pink pepper (GEO), essential oils of mature pink pepper (MEO), green tea extract, butylated hydroxyanisole (E320), butylated hydroxytoluene (E321), polyphenols (e.g., curcumins. curcuminoids, desmethoxycurcumin (hydroxycirmamoyl feruloylmethane), bis-desmethoxycurcumin), catechins (e.g., epigallocatechin gallate, epicatechin gallate, epigallocatechin, epicatechin, C catechin, catechins comprised in green tea extract), and derivatives and mixtures thereof.

Nonlimiting examples of carbohydrates include: monosaccharides, such as, for example, glucose, fructose, galactose, and mixtures thereof; disaccharides, such as, for example, maltose, lactose, sucrose, and mixtures thereof; polysaccharides, such as for example, starches (e.g., pectin, corn (maize) starch, oat starch, potato starch, sweet potato starch, rice starch, pea starch, w % beat starch, azuki starch, green bean starch, kudzu starch, Katakuri starch, arrowroot starch, mung bean starch, sago starch, tapioca starch, plant starch (e.g., starch obtained from any of the plants disclosed herein), and derivatives thereof, and mixtures of two or more thereof. In some embodiments, the starch is a modified starch (e.g., pregelatinized starch (e.g., corn, wheat, tapioca), pregelatinized high amylose content starch, pregelatinized hydrolyzed starches (e.g., maltodextrins, corn syrup solids, rice syrup solids, tapioca syrup solids), chemically modified starches such as pregelatinized substituted starches (e.g., octenyl succinate modified starches), alkaline modified starch, bleached starch, oxidized starch, monostarch phosphate, distarch phosphate, phosphated distarch phosphate, acetylated distarch phosphate, acetylated starch, mono starch acetate, acetylated starch, mono starch acetate, acetylated distarch adipate, distarch glycerine, hydroxy propyl starch, hydroxy propyl distarch glycerine, hydroxy propyl distarch phosphate, starch sodium octenyl succinate, acetylated oxidized starch, dextrin, sodium octenylsuccinate starch, and derivatives thereof, and mixtures of two or more thereof), flours (e.g., acorn flour, almond flour, amaranth flour, atta flour, barley flour, bean flour, buckwheat flour, cassava flour, chestnut flour, chuño flour, coconut flour, corn (maize) flour, durum flour, einkorn flour, emmer flour, fava bean flour, garbanzo flour, ground chia seeds, ground flaxseeds, hemp flour, khorasan flour, lentil flour, maida flour, malted barley flour, masa harina, mesquite flour, millet flour, nut flour, oat flour, pea flour, peanut flour, potato flour, quinoa flour, rice flour, rye flour, sorghum flour, soy flour, spelt flour, sweet rice flour, taro flour, teff flour, wheat flour, vital wheat gluten, ground chia seeds, ground flaxseed, and derivatives thereof, and mixtures of two or more thereof), gums (e.g., arrowroot flour, xanthan gum, acacia gum (gum arabic), gellan gum, guar gum, locust bean gum (carob gum), tragacanth gum, carrageenan, tara gum, wheat gum, konjac gum, agar gum, karaya gum, salep, modified cellulose (e.g., methylcellulose, methoxymethylcellulose, hydroxypropyl methylcellulose, carboxymethylcellulose, microcrystalline cellulose), and derivatives and mixtures thereof. In some embodiments, the gum is a modified gum (e.g., deacetylated, deacetylated clarified, partially deacetylated, partially deacetylated clarified, and derivatives thereof, and mixtures of two or more thereof)), edible fibers (e.g., acacia fiber, bamboo fiber, barley bran, carrot fiber, cellulose (e.g., wood pulp cellulose), citrus fiber, corn bran, soluble dietary fiber, insoluble dietary fiber, oat bran, pea fiber, rice bran, head husks, psyllium husk, konjac, soy fiber, soy polysaccharide, wheat bran, inulin, and derivatives thereof, and mixtures of two or more thereof), and mixtures of two or more thereof; and mixtures of two or more thereof.

Non-limiting examples of lipids include fats, oils, monoglycerides, diglycerides, triglycerides, phospholipids, and free fatty acids.

Non-limiting examples of oils include plant oils (e.g., sunflower oil, coconut oil, mustard seed oil, peanut oil, camelina sativa oil, canola oil, corn oil, cottonseed oil, cuphea oil, flax seed oil, olive oil, palm oil, rapeseed oil, safflower oil, sesame oil, soybean oil, almond oil, beech nut oil, brazil nut oil, cashew nut oil, hazelnut oil, macadamia nut oil, mongongo nut oil, pecan oil, pine nut oil, pistachio nut oil, walnut oil, avocado oil, grape oil), microbe-derived oils, algae-derived oils, fungus-derived oils, marine animal oils (e.g., Atlantic fish oil, Pacific fish oil, Mediterranean fish oil, light pressed fish oil, alkaline treated fish oil, heat treated fish oil, light and heavy brown fish oil, bonito oil, pilchard oil, tuna oil, sea bass oil, halibut oil, spearfish oil, barracuda oil, cod oil, menhaden oil, sardine oil, anchovy oil, capelin oil, Atlantic cod oil. Atlantic herring oil, Atlantic mackerel oil. Atlantic menhaden oil, salmonid oil, and shark oil, squid oil, cuttle fish oil, octopus oil, krill oil, seal oil, whale oil), non-essential oils, essential oils, natural oils, non-hydrogenated oils, partially hydrogenated oils, hydrogenated oils (e.g., hydrogenated coconut oil), crude oils, semi-refined (also called alkaline refined) oils, interesterified oils, and refined oils. In some embodiments, longer chain oils (e.g., sunflower oil, corn oil, olive oil, soy oil, peanut oil, walnut oil, almond oil, sesame oil, cottonseed oil, canola oil, safflower oil, flax seed oil, palm oil, palm kernel oil, palm fruit oil, coconut oil, babassu oil, shea butter, mango butter, cocoa butter, wheat germ oil, rice bran oil, engineered sunflower oil that over-expresses oleic acid by 400%) are combined with short-chain triglycerides to produce transesterified fatty acid esters (e.g., to create a specific flavor profile).

Non-limiting examples of monoglycerides and diglycerides include plant-derived monoglycerides and diglycerides, (e.g., monoglycerides and diglycerides derived from sunflower, coconut, peanut, cottonseed, olive, palm, rapeseed, safflower, sesame seed, soybean, almond, beech nut, Brazil nut, cashew, hazelnut, macadamia nut, mongongo nut, pecan, pine nut, pistachio, walnut, and avocado). The monoglycerides and diglycerides can comprise acyl chains of any free fatty acid known in the art, including acyl chains of any free fatty acid disclosed herein.

Non-limiting examples of free fatty acids include butyric acid, caproic acid, caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linolelaidic acid, α-linolenic acid, arachidonic acid, eicosapentaenoic acid, erucic acid, docosahexaenoic acid, omega-fatty acids (e.g., arachidonic acid, omega-3-fatty acids, omega-6-fatty acids, omega-7-fatty acids, omega-9-fatty acids), fatty acids with even number of carbons of 4-16 carbons in length, monosaturated acids (particularly with 18 carbons), fatty acids with low interfacial tension (e.g., less than 20, less than 15, less than 11, less than 9, less than 7, less than 5, less than 3, less than 2, less than 1, or less than 0.5 dynes/cm, from 0.1 to 20, from 1 to 15, from 2 to 9, from 3 to 9, from 4 to 9, from 5 to 9, from 2 to 7, from 0.1 to 5, from 0.3 to 2, or from 0.5 to 1 dynes/cm, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, or 20.0), butyric (4:0) acid or caproic (6:0) acid that is esterified at sn-3, medium-chain fatty acids (8:0-14:0) as well as 16:0 that are esterified at positions sn-1 and sn-2, fatty acids in which stearic acid (18:0) is placed at position sn-1, fatty acids in which oleic acid (18:1) is placed at positions sn-1 and sn-3, fatty acids that have a range of carbon atoms (e.g, from 8 to 40, from 10 to 38, from 12 to 36, from 14 to 34, from 16 to 32, from 18 to 30, or from 20 to 28 carbon atoms), fatty acids that comprise at least one unsaturated bond (i.e., a carbon carbon double or triple bond; e.g., at least 2, at least 3, at least 4, at least 5, at least 6, at least 7, or at least 8 carbon-carbon double bonds and/or triple bonds), fatty acids with conjugated unsaturated bonds (i.e., at least one pair of carbon-carbon double and/or triple bonds are bonded together, without a methylene (CH2) group between them (e.g., 4CH:CHi CH:CHi)), and derivatives of the above named fatty acids (e.g., esters (e.g., methyl and ethyl esters), salts (e.g., sodium and potassium salts), triglyceride derivatives, diglycerides derivatives, monoglyceride derivatives). The free fatty acids can be saturated or unsaturated. In some embodiments, the free fatty acids are not derived from or produced by a mammal.

Non-limiting examples of phospholipids include lecithin phospholipids (e.g., soy lecithin phospholipids, sunflower lecithin phospholipids, cotton lecithin phospholipids, rapeseed lecithin phospholipids, rice bran lecithin phospholipids, corn lecithin phospholipids, flour lecithin phospholipids), cardiolipin, ceramide phosphocholines, ceramide phosphoethanolamines, glycerophospholipids, phasphatidicacid, phosphatidylcholine, phosphatidylethanolamine, phosphatidylinositol, phosphospingolipids, and phsophatidylserine. In some embodiments, the phospholipids are not derived from or produced by a mammal.

Non-limiting examples of triglycerides include tributyrin, short-chain triglycerides, short-chain triglycerides comprising three oleic acids; short-chain triglycerides comprising hexanoic acid; short-chain triglycerides comprising hexanoic acid and butyric acid; short-chain triglycerides comprising hexanoic acid and decanoic acid; and short-chain triglycerides comprising one butyric, one hexanoic, and one octanoic acid.

Non-limiting examples of essential and semi-essential amino acids include cysteine, methionine, isoleucine, leucine, phenylalanine, tryptophan, and valine.

Non-limiting examples of functional agents include acidulants, buffering agents, shelf life extending agents, pH and/or ionic strength adjusting agents, anti-microbial agents, anti-oxidants, preservatives, emulsifiers, plasticizers, texturing/mouthfeel agents, coloring agents, taste/flavor agents, aroma agents, leavening agents, and flow agents.

Non-limiting examples of shelf life extending agents include carbon monoxide, nitrites, sodium metabisulfite, Bombal, and derivatives and mixtures thereof.

Non-limiting examples of preservatives include p-hydroxybenzoate derivatives, sorbic acid, benzoic acid, nisin, natamycin, and derivatives and mixtures thereof.

Non-limiting examples of emulsifiers include anionic emulsifiers, non-ionic emulsifiers, cationic emulsifiers, amphoteric emulsifiers, bioemulsifiers, steric emulsifiers, Pickering emulsifiers, glycolipids (e.g., trehalose lipids, sophorolipids, rhamnolipids, mannosylerythriol lipids), oligopeptides (e.g., gramicidin S, polymyxin), lipopeptides (e.g., surfactin), phospholipids, fatty acids, neutral lipids, polymeric biosurfactants, amphipathic polysaccharides, lipopolysaccharides, proteins (e.g., pea protein, soy protein, chickpea protein, algae protein, yeast protein, potato protein, lentil protein), mannoprotein, sodium phosphates, calcium stearoyl lactylate, mono- and diacetyl tartaric acid esters of monoglycerides, phospholipids, sorbitan monostearate, magnesium stearate, sodium/potassium/calcium salts of fatty acids, calcium stearoyl di lactate, poly-glycerol esters, sorbitan fatty acid esters, acetic acid esters of monoglycerides, lactic acid esters of monoglycerides, citric acid esters of monoglycerides, polyglycerol esters of fatty acids, polyglycerol polyricinoleate, propane-1,2-diol esters of fatty acids, sugar esters, sucrose esters of fatty acids, monoglycerides, acetylated monoglycerides, lactylated monoglycerides, diglycerides, phosphate monoglycerides, diacetyl tartaric acid esters, sodium/calcium stearoyl-2-lactylate, ammonium phosphatide, polysorbates, polysorbate-80, carboxymethylcellulose (CMC), modulated cellulose, citric acid esters, locust bean gum, guar gum, liposan, emulsan, lecithins (e.g., plant-based lecithins, garbanzo lecithin, fava bean lecithin, soy lecithin, sunflower lecithin, canola lecithin), surfactants (e.g., sorbitan trioleate (Span 85, respectively), sorbitan tristearate (Span 65, respectively), sorbitan sesquioleate (Arlacel 83), glyceryl monostearate, sorbitan monooleate (Span 80), sorbitan monostearate (Span 60), sorbitan monopalmitate (Span 40), sorbitan monolaurate (Span 20), polyoxyethylene sorbitan tristearate (Tween 65, respectively), polyoxyethylene sorbitan trioleate (Tween 85, respectively), polyethylene glycol 400 monostearate, polysorbate 60 (Tween 60), polyoxyethylene monostearate, polysorbate 80 (Tween 80), polysorbate 40 (Tween 40), polysorbate 20 (Tween 20), PEG 20 tristearate, PEG 20 trioleate, PEG 20 monostearate, PEG 20 monooleate, PEG 20 monopalmitate, and PEG 20 monolaurate sorbitan), and derivatives and mixtures thereof.

Non-limiting examples of plasticizers include diethanolamin, triethanolamine, glycerol, sorbitol, PEG-300, PEG-600, urea, octanoic acid, palmitic acid, dibutyl tartrate and phthalate, mono-, di-, or triglycerides esters, fructose, caproic acid, hydrocaproic acid, di-, tri- or tetra-ethylene glycol, glycerol, 1,3-propane diol, 1,4-butane diol, 1,5-pentane diol, sucrose, and derivatives and mixtures thereof.

Non-limiting examples of texturing/mouthfeel agents include gums (e.g., guar gum, carob gum, wheat gum, xanthan gum), bulking agents, fillers, anti-adherent compounds, dispersing agents, moisture absorbing compounds, chemesthetic agents, film-forming agents, thickening agents, hardening agents, softening agents, stabilizers, anti-caking agents, anti-foaming agents, and derivatives and mixtures thereof.

Non-limiting examples of flavor/aroma agents include ethyl butyrate, 2-furyl methyl ketone, 2,3-pentanedione, γ-undecalactone, 8-undecalactone, propylene glycol, glycerol, ethyl alcohol, dimethylsulfide, 2-methylbutanol, 4-cis-heptenal 2-trans-nonenal, acetone, 2-undecanone, 2-butanone, amyl alcohol, S-decalactone, 2-heptanone, 8-dodecalactone, 2-nonanone, S-tetradecalactone, hydrogen sulfide, dimethyl sulfone, benzothiazole, 2-pentanone, 2-tridecanone, S-octalactone, 2-pentadecanone, natural favors, artificial flavors (e.g., chocolate flavoring, coffee flavoring, strawberry flavoring, almond flavoring, hazelnut flavoring, vanilla flavoring, green tea flavoring, Irish cream flavoring, coconut flavoring), sweetening agents (e.g., non-protein sweetening agents, protein-based sweetening agents), and derivatives and mixtures thereof.

Non-limiting examples of non-protein sweetening agents include sugars, modified sugars, natural sweeteners, sweet proteins, artificial sweeteners, sugar alcohols, sugar fibers, sugar extracts including: sucrose, cane juice, corn sugar, high fructose corn syrup, corn sweetener, agave syrup, barley malt syrup, birch syrup, blackstrap molasses, brown rice syrup, caramel, corn sugar, dextrose, douxmatok syrup, coconut palm sugar, fructose, galactose, glucose, glucose fructose syrup, golden syrup, acesulfame potassium, advantame, alitame, aspartame, aspartame-acesulfame salt, cyclamates (e.g., sodium cyclamate), erythritol, fructooligosaccharides, allulose, glucitol (sorbitol), glycerol (glycerin), glycyrrhizin, golden syrup, HFCS-42, HFCS-55, HFCS-90, high maltose corn syrup, honey, date syrup, HSH, hydrogenated starch hydrolysate (HSH), isomaltulose, isomalto-oligosaccharide (IMO), isoglucose, inulin, inverted sugar, isomalt, lactitol, lactose, levulose (fructose), luo han guo (aka monk fruit), maltitol, maltodextrin, maltose, mannitol, maple syrup, molasses, monatin, monellin, neohesperidin dihyrdochalcone, neotame, oligofructose, palm sugar, polydextrose, rapadura, refiners syrup, saccharin, saccharose, sorghum Syrup, stevia, RebM, RebA, RebD, stevioside, sucralose, sucrose, tagatose, osladin, dulcin, glucin, asulfame potassium, L-aspartyl-L-phenylalanine, P-4000, mogrosides, trehalose, xylitol, yacon syrup, and derivatives and mixtures thereof.

Non-limited examples of protein-based sweetening agents include brazzein (UniProt sequence P56552), curculin (UniProt sequence P19667 amino acids 23 to 136, Q6F495 amino acids 23 to 135, respectively), mabinlin (UniProt sequences P80351 amino acids 1 to 32, P80351 amino acids 33 to 104, P30233 amino acids 36 to 68, P30233 amino acids 83 to 154, P80352 amino acids 1 to 32, P80352 amino acids 33 to 104, P80353 amino acids 1 to 28, P80353 amino acids 29 to 100), miraculin (UniProt sequence P13087 amino acids 30 to 220), monelin (UniProt sequences P02881, P02882), pentadin, and thaumatin (UniProt sequences P02883 amino acids 23 to 229, P02884 amino acids 23 to 229, respectively), and homologs and fragments and mixtures thereof.

### Attributes

The composition according to any of the above can have an attribute (e.g., any one of the attributes disclosed herein, or combination of two or more of the attributes disclosed herein) that is between 50% and 150%, 140%, 130%, 120%, 110%, 100%, 90%, 80%, 70%, or 60%; between 60% and 150%, 140%, 130%, 120%, 110%, 100%, 90%, 80%, or 70%; between 70% and 150%, 140%, 130%, 120%, 110%, 100%, 90%, or 80%; between 80% and 150%, 140%, 130%, 120%, 110%, 100%, or 90%; between 90% and 150%, 140%, 130%, 120%, 110%, or 100%; between 100% and 150%, 140%, 130%, 120%, or 110%; between 110% and 150%, 140%, 130%, or 120%; between 120% and 150%, 140%, or 130%; between 130% and 150%, or 140%; or between 140% and 150% of such attribute of a corresponding composition, or that is at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, or at least 20-fold of such attribute of a corresponding composition. The recombinant milk protein according to any of the above comprised in the composition can impart or materially contribute to such attribute of the composition.

The attribute can be one or more physical attributes, one or more mechanical attributes, one or more chemical/biological attributes, one or more sensory attributes, or one or more functional attributes, or any combination thereof.

Non-limiting examples of physical attributes include appearance (e.g., browning, color, translucence, opaqueness), shape (e.g., length, width, uniformity), shape retention, structure (e.g., molecular structure [e.g., protein folding/conformation], air cell average size, air cell size distribution, air cell wall thickness), crystallinity (e.g., providing for a specific translucence, opaqueness, or transparency; for example, a higher crystallinity generally allows less light to pass through a composition, affecting translucence or opaqueness of the composition, as well as mechanical strength, stiffness, chemical resistance, and stability), layering, aeration, content/size/shape of solid particles, hardness/firmness, cohesion, plasticity, viscosity, density, solubility (e.g., solubility in various solvents, gelling point/profile (i.e., temperature and timeline at which gel formation sets in), and melting point/profile.

Non-limiting examples of mechanical attributes include hardness/firmness, adhesiveness, resilience/recoverable energy, structural integrity/cohesiveness, elasticity/springiness/rebound, and chewiness/breakdown.

Non-limiting examples of functional attributes include foaming/leavening behavior (e.g., foaming capacity (i.e., capacity to form a foam, wherein the term “foam” as used herein refers to air bubbles dispersed in a solid or aqueous continuous phase; as measured, for example, by overrun and/or air phase volume), foam strength (measured, for example, as yield stress under shear or the amount of stress required to initiate flow in the sample), foam stability (i.e., half-life of foam in response to a physical and/or chemical condition), foam drainage (i.e., rate at which a foam destabilizes and an aqueous phase begins to drain from the foam), foam seep), gelling/thickening/coagulating behavior (e.g., gelling/thickening capacity (i.e., capacity to form a gel, wherein the term “gel” as used herein refers to a protein network with spaces filled with solvent linked by hydrogen bonds to the protein molecules) having defined viscoelastic properties, as measured, for example, by the storage and elastic moduli and phase angle obtained in frequency sweeps on a rheometer or by resistance to a physical and/or chemical condition (e.g., agitation, temperature, pH, ionic strength, protein concentration, sugar concentration, ionic strength), gelling profile (e.g., curve of gelling capacity over time, viscoelastic parameters as a function of temperature), gel strength (i.e., mechanical force required to break a gel surface of a defined area, as measured, for example, by the storage modulus obtained in frequency sweeps on a rheometer), water holding capacity upon gelling, syneresis upon gelling (i.e., water weeping over time)), emulsifying behavior (e.g., emulsifying capacity (i.e., capacity to stabilize an emulsion or the amount of oil that a given mass of sample can emulsify without destabilization), emulsion stability (i.e., half-life of an emulsion produced under given conditions, such as, for example, a given protein concentration, lipid concentration, pH, ionic strength, or preparation method)), water binding behavior (e.g., water binding capacity (i.e., capacity to bind water), water binding strength), syneresis upon gelling (i.e., water weeping over time), aggregation behavior (e.g., aggregation capacity (i.e., capacity to form a precipitate (i.e., a tight protein network based on strong interactions between protein molecules and exclusion of solvent), as measured, for example, by resistance to a physical and/or chemical condition), aggregation capacity over time (i.e., curvy of aggregation capacity over time), aggregate stability (e.g., upon heating, at various pHs, at various ion concentrations), ability to form micelles (i.e., generally or roughly spherical supramolecular structure that exist as a dispersion within a composition and that can encapsulate one or more biomolecules [e.g., water, minerals, vitamins)), interaction with other proteins (e.g., other milk proteins), crystallization, lubricity; spreadability; and use versatility (i.e., potential for varied use and production of a diversity of compositions; e.g., ability to produce food products that resemble milk derivative products [e.g., any of the milk derivative products disclosed herein]).

Non-limiting examples of chemical/biological attributes include biodegradability (e.g., biodegradability under aerobic or unaerobic conditions), biocompatibility, nutrient content (e.g., types and/or amounts of proteins, types and/or amounts of amino acids [e.g., branched amino acids], PDCAAS, BV, types and/or amounts of lipids, types and/or amounts of minerals, types and/or amounts of vitamins). pH, digestibility (e.g., gastrointestinal digestibility), absorption (e.g., proportion of absorbed protein from a food product), oxidation stability, ability to grow (e.g., comprising non-polymerized monomers that polymerize over time or under specific conditions [e.g., temperature, oxygenation, pH, pressure, shear]), and hunger and/or satiety regulation.

Non-limiting examples of chemical/biological attributes include flavor, aroma, and eating quality (e.g., mouthfeel, fattiness, creaminess, richness, greasiness, thickness, hardness/firmness, crispiness, crumbliness, crunchiness, chewiness, chewdown, tenderness, compactness, cohesiveness, adhesiveness, graininess, smoothness, juiciness, wetness, mouthcoating, slipperiness on tongue, roughness, abrasiveness, uniformity of bite and/or chew, springiness, texture, airiness, effort required to draw sample from spoon over tongue).

Methods for measuring such attributes are known in the art, and include, for example, qualitative analysis (e.g., assessment of appearance or sensory attributes by human sensory experts; viscosity analysis by rate or ease of flow or ease of movement during handling), or quantitative analysis [e.g., determination of protein folding/conformation by melting temperature analysis [e.g., comparing recombinant milk protein to native milk protein using Therme Shift Assay (see, for example, Pantoliano et al. 2001 J Biomol Screen 6:429-40)] or by analyzing protein binding to known ligands (e.g., retinol, palmitate (see, for example, Yang et al. 2007 Proteins 71:1197-1210)]; measurement of crystallinity using differential scanning calorimetry (DSC) [B. Wunderlich, Thermal Analysis, Academic Press, 1990, pp. 417-431.; TN 48, “Polymer Heats of Fusion”, TA Instruments. New Castle, De]; color analysis by spectroscopic measurement in L*a*b* color space; structure analysis by microscopic examination; viscosity analysis by viscometric or rheometric methods [e.g., rotational viscometric methods, capillary viscometric methods, vibratory viscometric methods, ultrasonic pulse echo methods, pycnometric methods, or areometric methods (see, for example, Kazys & Rekuviene 2011 Ultragarsas (Ultrasound) 66(4):20-25)]; density analysis by densymetric methods; melting point analysis by calorimetry; analysis of mechanical attributes using a texture analyzer [see, for example, PCT publication WO2020219595, published Oct. 29, 2020]; analysis of biodegradability [see, for example, OECD 306 Biodegradability in Sea Water, OECD 311 (ASTM E2170) Anaerobic Biodegradability/Biochemical Methane Potential, ASTM D5338 Aerobic biodegradability/composting assay, ASTM D5511 Anaerobic biodegradability/“landfill simulation”, ASTM D5988 (ISO17556) Biodegradation in soil]; analysis of nutrient content by AOAC International reference methods AOAC 990.03 and AOAC 992.15, electrophoresis (e.g., SDS-PAGE), liquid column chromatography, immunochemical tests, or on-chip electrophoresis (e.g., using the Agilent Protein 80 kit and the Agilent 2100 Bioanalyzer) for determination of type and/or content of proteins and amino acids; calculation of nutrient content from nutrient contents of ingredients; analysis of foaming/leavening behavior by measurement of percentage of air incorporated in a foam formed after whipping at a specified speed and for a specified amount of time under defined conditions [e.g., temperature, pH, ionic strength, protein concentration, carbohydrate concentration], measurement of how long it takes for a given mass of foam to destabilize in the form of liquid draining or seeping, measurement of yield stress under shear or the amount of stress required to initiate flow in a sample [see, for example, PCT publication WO2020219595, published Oct. 29, 2020]; analysis of gelling/thickening/coagulating behavior by measurement of the time required to form a gel under defined conditions [e.g., temperature, pH, ionic strength, protein concentration, carbohydrate concentration], measurement of storage and elastic moduli and phase angle obtained in frequency sweeps on a rheometer, and measurement of resistance of a gel to a physical force and/or chemical condition [e.g., agitation, temperature, pH, ionic strength, protein concentration, sugar concentration, ionic strength] [see, for example, PCT publication WO2020219595, published Oct. 29, 2020]; analysis of emulsifying behavior by preparation of a lipid in water emulsion under defined conditions [e.g., mixing apparatus, mixing speed, mixing time] and subsequent measurement of stability over time of phase separation in a mixture of lipid and water, measurement of rate of creaming or sedimentation, measurement of change in opacity over time, or measurement of change of dispersed phase particle size over time; analysis of water binding behavior by measurement of amount of water exuded after centrifugation, or development of moisture sorption isotherms based on mass of water bound per mass of protein as a function of vapor pressure).

### Food Product

The composition according to any of the above can be a food product, wherein the food product comprises a milk protein component that comprises or consists of the recombinant milk protein according to any of the above, and wherein the food product has an attenuated or essentially eliminated allergenicity compared to a corresponding food product.

Food products comprising β-lactoglobulin and/or α-lactalbumin are desirable, particularly for athletes, as these milk proteins have high contents of essential and branched-chain amino acids, which are thought to aide production of muscle tissue. Moreover, pi-lactoglobulin is desirable as a food additive as it has good water binding ability, which property makes β-lactoglobulin suitable for managing water activity of food products. Moreover, β-lactoglobulin is desirable as a food additive as it has anti-microbial activity, which property makes β-lactoglobulin suitable for extending the shelf life of food products. Moreover. β-lactoglobulin is desirable as a food additive as it can readily absorb at interfaces, which property makes β-lactoglobulin suitable for producing highly stable dispersions in food products. Moreover, α-lactalbumin is rich in the amino acid cysteine, which is a building block of glutathione, a powerful antioxidant in the body that plays an important role in immunity, and the neurotransmitter serotonin and the neurosecretory hormone melatonin, which play a role in regulating neurobehavioral effects such as appetite, sleeping-waking rhythm, pain perception, mood, anxiety and stress control. Moreover, food products comprising non-allergenic milk proteins are desirable as they can be consumed by an increasing number of people who are allergic against dairy products.

The food product can be a food product, or can resemble a food product (i.e., can be a “substitute food product”), selected from any of the food product categories defined by the National Health and Nutrition Examination Survey (NHANES).

Non-limiting examples of NHANES food product categories include snack foods and gums (e.g., snack bars, crackers, salty snacks from grain products, chewing gums); breads, grains, and pastas (e.g., oat breads and rolls, cornbread, corn muffins, tortillas, flour and dry mixes, biscuits, multi-grain breads and rolls, whole wheat breads and rolls, pastas, rye breads and rolls, cracked wheat breads and rolls, white breads and rolls); beverages (e.g., beers and ales, beverage concentrates, beverages, energy drinks, sports drinks, fluid replacements, soft drinks, carbonated beverages, juices, wines, beers, cocktails, nutrition drinks, nutrition powders, protein-enriched beverages, coffee, tea); sweets and desserts (e.g., cakes, candies, chips, cookies, cobblers, pastries, ices or popsicles, muffins, pies, sugar replacements or substitutes, syrups, honey, jellies, jams, preserves, salads, crepes, Danish, breakfast pastries, doughnuts); breakfast foods (e.g., cereal grains, cereal, rice, French toast, pancakes, waffles, coffee cake); salad dressings, oils, sauces, condiments (e.g., cooking fats, vegetable oils, salad dressings, tomato sauces, gravies); potatoes (e.g., potato salad, potato soups, chips and sticks, fried potatoes, mashed potatoes, stuffed potatoes, puffs); and soups (e.g., vegetable soups, vegetable broths), meals, main dishes, proteins (e.g., meat substitutes), and seafoods.

The food product according to any of the above can be a dairy product, a supplemented dairy product (i.e., a conventional dairy product that is supplemented with the recombinant milk protein according to any of the above), or substitute dairy product (i.e., a food product that resembles a conventional dairy product). The term “dairy product” as used herein refers to milk (e.g., whole milk [at least 3.25% milk fat], partly skimmed milk [from 1% to 2% milk fat], skim milk [less than 0.2% milk fat], cooking milk, condensed milk, flavored milk, goat milk, sheep milk, dried milk, evaporated milk, milk foam), and products derived from milk, including but not limited to yogurt (e.g., whole milk yogurt [at least 6 grams of fat per 170 g], low-fat yogurt [between 2 and 5 grams of fat per 170 g], nonfat yogurt [0.5 grams or less of fat per 170 g], greek yogurt [strained yogurt with whey removed], whipped yogurt, goat milk yogurt, Labneh [labne], sheep milk yogurt, yogurt drinks [e.g., whole milk Kefir, low-fat milk Kefir], Lassi), cheese (e.g., whey cheese such as ricotta; pasta filata cheese such as mozzarella; semi-soft cheese such as Havarti and Muenster; medium-hard cheese such as Swiss and Jarlsberg and halloumi; hard cheese such as Cheddar and Parmesan; washed curd cheese such as Colby and Monterey Jack; soft ripened cheese such as Brie and Camembert; fresh cheese such as cottage cheese, feta cheese, cream cheese, paneer, and curd), processed cheese, processed cheese food, processed cheese product, processed cheese spread, enzyme-modulated cheese; cold-pack cheese), dairy-based sauces (e.g., salad dressing, bechamel sauce, fresh sauces, frozen sauces, refrigerated sauces, shelf stable sauces), dairy spreads (e.g., low-fat spread, low-fat butter), cream (e.g., dry cream, heavy cream, light cream, whipping cream, half-and-half, coffee whitener, coffee creamer, sour cream, creme fraiche), frozen confections (e.g., ice cream, smoothie, milk shake, frozen yogurt, sundae, gelato, custard), dairy desserts (e.g., fresh, refrigerated, or frozen), butter (e.g., whipped butter, cultured butter), dairy powders (e.g., whole milk powder, skim milk powder, fat-filled milk powder (i.e., milk powder comprising plant fat in place of all or some animal fat), infant formula, milk protein concentrate (i.e., protein content of at least 80% by weight; e.g., milk protein concentrate, whey protein concentrate, demineralized whey protein concentrate. β-lactoglobulin concentrate. α-lactalbumin concentrate, glycomacropeptide concentrate, casein concentrate), milk protein isolate (i.e., protein content of at least 90% by weight; e.g., milk protein isolate, whey protein isolate, demineralized whey protein isolate, β-lactoglobulin isolate, α-lactalbumin isolate, glycomacropeptide isolate, casein isolate), nutritional supplements, texturizing blends, flavoring blends, coloring blends, ready-to-drink or ready-to-mix products (e.g., fresh, refrigerated, or shelf stable dairy protein beverages, weight loss beverages, nutritional beverages, sports recovery beverages, and energy drinks), puddings, gels, chewables, crisps, bars (e.g., nutrition bars, protein bars), and fermented dairy products (e.g., yoghurt, cheese, sour cream, cultured buttermilk, cultured butter, cultured butter oil).

The food product according to any of the above can be an animal meat or animal meat product, a supplemented animal meat or animal meat product (i.e., a conventional animal meat or animal meat product that is supplemented with the recombinant milk protein according to any of the above produced by the recombinant host cell according to any of the above and/or a method according to any of the above), or substitute animal meat or animal meat product (i.e., a food product that resembles a conventional animal meat or animal meat product). Non-limiting examples of animal meats and animal meat products include flesh obtained from skeletal muscle or from other organs (e.g., kidney, heart, liver, gallbladder, intestine, stomach, bone marrow, brain, thymus, lung, tongue), or parts thereof, obtained from an animal. The animal meat can be dark or white meat. Non-limiting examples of animals from which animal meat or animal meat product can be obtained include cattle, lamb, mutton, horse, poultry (e.g., chicken, duck, goose, turkey), fowl (e.g., pigeon, dove, grouse, partridge, ostrich, emu, pheasant, quail), fresh or salt water fish (e.g., catfish, tuna, spearfish, shark, halibut, sturgeon, salmon, bass, muskie, pike, bowfin, gar, eel, paddlefish, bream, carp, trout, walleye, snakehead, crappie, sister, mussel, scallop, abalone, squid, octopus, sea urchin, cuttlefish, tunicate), crustacean (e.g., crab, lobster, shrimp, barnacle), game animal (e.g., deer, fox, wild pig, elk, moose, reindeer, caribou, antelope, zebra, squirrel, marmot, rabbit, bear, beaver, muskrat, opossum, raccoon, armadillo, porcupine, bison, buffalo, boar, lynx, bobcat, bat), reptile (e.g., snakes, turtles, lizards, alligators, crocodiles), any insect or other arthropod, rodent (nutria, guinea pig, rat, mice, vole, groundhog, capybara), kangaroo, whale, and seal. The animal meat or animal meat product can be ground, chopped, shredded, or otherwise processed, and uncooked, cooking, or cooked.

The food product according to any of the above can be an egg or egg product, a supplemented egg product (i.e., a conventional egg or egg product that is supplemented with the recombinant milk protein according to any of the above), or substitute egg or egg product (i.e., a food product that resembles a conventional egg or egg product). Non-limiting examples of eggs or egg products include whole egg (e.g., liquid whole egg, spray-dried whole egg, frozen whole egg), egg white (e.g., liquid egg white, spray-dried egg white, frozen egg white), egg yolk, egg dishes, egg soups, mixtures made with egg whites, mixtures made with egg substitutes, mayonnaise, custard, and salad dressings.

Resemblance of the substitute food product provided herein to a conventional food product can be due to any physical attribute (e.g., any physical attribute disclosed herein), mechanical attribute (e.g., any mechanical attribute disclosed herein), chemical/biological attribute (e.g., any chemical/biological attribute disclosed herein), sensory attribute (e.g., any sensory attribute disclosed herein), and functional attribute (e.g., any functional attribute disclosed herein), and any combination thereof.

### Polymer

The composition according to any of the above can comprise a polymer or polymer network (i.e., a network of polymers that are linked with each other) comprising linked repeated protein monomers, wherein the repeated protein monomers comprise or consist of the recombinant milk protein according to any of the above.

The repeated protein monomers comprised in the polymer or polymer network, and/or the polymers comprised in the polymer network, can be linked directly with each other or via intermediary molecules. The repeated protein monomers comprised in the polymer or polymer network, and/or the polymers comprised in the polymer network, can be linked via covalent bonds (amide bonds [e.g., lactam bridges, native chemical ligation bonds, Staudinger ligation bonds], disulfide bonds) or non-covalent bonds (e.g., electrostatic interactions, hydrogen bonds).

The composition can comprise between 0.001% and 100%, 99%, 95%, 90%, 80%, 70%, 60%, 50%, 40%, 30%, 20%, 10%, 1%, 0.1%, or 0.01%; between 0.01% and 100%, 99%, 95%, 90%, 80%, 70%, 60%, 50%, 40%, 30%, 20%, 10%, 1%, or 0.1%; between 0.1% and 100%, 99%, 95%, 90%, 80%, 70%, 60%, 50%, 40%, 30%, 20%, 10%, or 1%; between 1% and 100%, 99%, 95%, 90%, 80%, 70%, 60%, 50%, 40%, 30%, 20%, or 10%; between 10% and 100%, 99%, 95%, 90%, 80%, 70%, 60%, 50%, 40%, 30%, or 20%; between 20% and 100%, 99%, 95%, 90%, 80%, 70%, 60%, 50%, 40%, or 30%; between 30% and 100%, 99%, 95%, 90%, 80%, 70%, 60%, 50%, or 40%; between 40% and 100%, 99%, 95%, 90%, 80%, 70%, 60%, or 50%; between 50% and 100%, 99%, 95%, 90%, 80%, 70%, or 60%; between 60% and 100%, 99%, 95%, 90%, 80%, or 70%; between 70% and 100%, 99%, 95%, 90%, or 80%; between 80% and 100%, 99%, 95%, or 90%; between 90% and 100%, 99%, or 95%; between 95% and 100% or 99%, or between 99% and 100% by mass of the polymer or polymer network.

The polymer or polymer network comprised in the composition can comprise between 0.001% and 100%, 99%, 95%, 90%, 80%, 70%, 60%, 50%, 40%, 30%, 20%, 10%, 1%, 0.1%, or 0.01%; between 0.01% and 100%, 99%, 95%, 90%, 80%, 70%, 60%, 50%, 40%, 30%, 20%, 10%, 1%, or 0.1%; between 0.1% and 100%, 99%, 95%, 90%, 80%, 70%, 60%, 50%, 40%, 30%, 20%, 10%, or 1%; between 1% and 100%, 99%, 95%, 90%, 80%, 70%, 60%, 50%, 40%, 30%, 20%, or 10%; between 10% and 100%, 99%, 95%, 90%, 80%, 70%, 60%, 50%, 40%, 30%, or 20%; between 20% and 100%, 99%, 95%, 90%, 80%, 70%, 60%, 50%, 40%, or 30%; between 30% and 100%, 99%, 95%, 90%, 80%, 70%, 60%, 50%, or 40%; between 40% and 100%, 99%, 95%, 90%, 80%, 70%, 60%, or 50%, between 50% and 100%, 99%, 95%, 90%, 80%, 70%, or 60%; between 60% and 100%, 99%, 95%, 90%, 80%, or 70%; between 70% and 100%, 99%, 95%, 90%, or 80%; between 80% and 100%, 99%, 95%, or 90%; between 90% and 100%, 99%, or 95%; between 95% and 100% or 99%; or between 99% and 100% by mass of the recombinant milk protein.

The composition can be a supplemented polymer that is produced by mixing one or more petroleum-derived monomers with protein monomers that comprise or consist of the recombinant milk protein according to any of the above. The mass ratio of the monomers that comprise or consist of the recombinant milk protein to the petroleum-derived monomers can be between about 1 to 100 and about 100 to 1 (e.g., about 100 to 1, about 90 to 1, about 80 to 1, about 70 to 1, about 60 to 1, about 50 to 1, about 40 to 1, about 30 to 1, about 20 to 1, about 10 to 1, about 9 to 1, about 8 to 1, about 7 to 1, about 6 to 1, about 5 to 1, about 4 to 1, about 3 to 1, about 2 to 1, about 1 to 1, about 1 to 2, about 1 to 3, about 1 to 4, about 1 to 5, about 1 to 6, about 1 to 7, about 1 to 8, about 1 to 9, about 1 to 10, about 1 to 20, about 1 to 30, about 1 to 40, about 1 to 50, about 1 to 60, about 1 to 70, about 1 to 80, about 1 to 90, or about 1 to 100).

Non-limiting examples of suitable polymers include adhesives (i.e., a material that forms an adhesive bond; e.g., glue, wallpaper adhesive, wood adhesive, paper adhesive, cork adhesive, chipboard adhesive, surgical/medical glue, cement, mucilage, paste), coatings or facings (e.g., glossy coating, protective coating, varnish, coating for medical tablet, paper coating, painting, leather finishing, textile coating), paints or inks or pigment binders for ink, hard plastics (e.g., bottle, button, window, pen), medium hard plastics (e.g., bottle, fiber [e.g., yam], textile, carpet, curtain, clothing), soft plastics (e.g., bag, wrap, edible film, waterproof film, contact lens, packaging material), fabrics (e.g., textile, carpet, curtain), industrial polymers (i.e., compounds used in the manufacture of synthetic industrial materials), pharmaceutical formulations (e.g., product used for delivery of a medicinal agent (e.g., micro- or nano-particle (e.g., bead, micelle) that encapsulates a therapeutic or nutraceutical for delivery (e.g., controlled delivery), coating of tablet, capsule, compact, hydrogel), medical diagnostics (see, for example, J. Berger et al. 2004. Europ J of Pharm and Biopharm 57:19, respectively), gels (e.g., hydrogel for controlled release of a therapeutic, hydrogel for immobilizing a protein (e.g., enzyme)), implants (e.g., bone-replacing composite, material supporting nerve repair, scaffold for growing cells, prosthetic implant), articles of clothing (e.g., shoe), lubricants, pieces of furniture, cosmetics or personal care products (e.g., ointment, lotion, cream (e.g., moisturizing cream), cleanser, massage cream, soap, hair shampoo, hair conditioner, skin mask, finishing product, hair tonic), papers (e.g., paper sheet, paper label, packaging paper, photographic support), household items (e.g., pot, bowl, plate, cup), and biological scaffolds (i.e., a structure that mimics a biological matrix, sutures, bone-replacing material, material supporting nerve repair, scaffold for growing cells, prosthetic implant, membrane for promoting wound healing, tissue-engineering scaffolding).

The polymer according to any of the above can resemble a conventional petroleum-derived polymer. Such resemblance can be due to a similar color, shape (e.g., length, width, uniformity), shape retention, strength of adhesiveness, reaction to moisture, allergenicity, charge, hydrophobicity, hydrophilicity, texture, thickness, smoothness, hardness, tensile strength, digestibility, solvation, chemical reactivity, permeability, melting temperature, brittleness, toughness, creep or cold flow, porosity, swelling, barrier resistance, impact resistance, gas permeability, electrical conductivity, thermal conductivity, elastic modulus, flexibility, release of an associated/bound compound, gas content, strength-at-break, glass transition temperature, shaping temperature, crystallinity (e.g., translucence, opaqueness, transparency), and viscosity and/or density.

### Method for Producing Composition

In another aspect, provided herein is a method for producing a composition according to any of the above (e.g., food product according to any of the above, composition comprising a polymer or polymer network according to any of the above), wherein the method comprises the step of obtaining the recombinant milk protein according to any of the above.

When the composition is a food product (e.g., the food product according to any of the above), a variety of recipes known in the art can be used to prepare the food product. The recombinant milk according to any of the above can be used in such recipes in purified/isolated form or comprised in a fermentation broth or preparation obtained in a method according to any of the above.

When the composition comprises a polymer or polymer network according to any of the above, a variety of methods for polymerizing protein monomers are known in the art, and can be used to polymerize the recombinant milk protein according to any of the above. Non-limiting examples of such methods include methods that employ crosslinking agents (i.e., chemicals that activate functional groups on proteins and thus connect proteins without incorporating a spacer), crosslinking enzymes (e.g., transferases [enzyme commission number (EC) 2; e.g., transglutaminases], hydrolases [EC 3], oxidation (e.g., using oxidizing agents), reduction (e.g., using reducing agents), radiation (e.g., using UV, gamma, electron beam), heating, mechanical agitation, pressure (e.g., extrusion), turbulence, friction, pH changes, photo-oxidative treatment (e.g., using photo-reactive amino acid analogs), 3D-printing, and combinations thereof (see, for example, PCT publication WO2019213155).

The methods can further comprise the step of adding, at any step of the method, one or more other milk proteins (e.g., any of the other milk proteins disclosed herein), and/or one or more other ingredients (e.g., any of the other ingredients disclosed herein).

The method can further comprise the step of pre-digesting (i.e., digesting prior to use) the recombinant milk using a protease (e.g., any of the proteases disclosed herein that can cleave the recombinant milk at a native protease recognition or cleavage sequence or at a non-native protease recognition or cleavage sequence [e.g., any of the non-native protease recognition or cleavage sequences disclosed herein]). Efficacy of such pre-digestion can be increased by prior heat treatment of the recombinant milk, and/or by exposure of the recombinant milk to an acidic pH, to denature the recombinant milk and thereby make the native or non-native protease recognition or cleavage sequence more accessible to the protease. Pre-digestion can be followed by treatment with a transglutaminase such that the recombinant milk fragments are polymerized. (See, for example, Damodaran & Li 2017 Food Chemistry 237:724-732.)

### EXAMPLES

The following examples are included to illustrate specific embodiments of the invention. The techniques disclosed in the examples represent techniques discovered by the inventors to function well in the practice of the invention, however, those of skill in the art should, in light of the present disclosure, appreciate that many changes can be made in the specific embodiments that are disclosed and still obtain a like or similar result without departing from the spirit and scope of the invention. Therefore, all matter set forth or shown in the examples is to be interpreted as illustrative and not in a limiting sense.

### Example 1: Production of Recombinant i-Lactoglobulin Proteins Having Attenuated or Essentially Eliminated Allergenicity

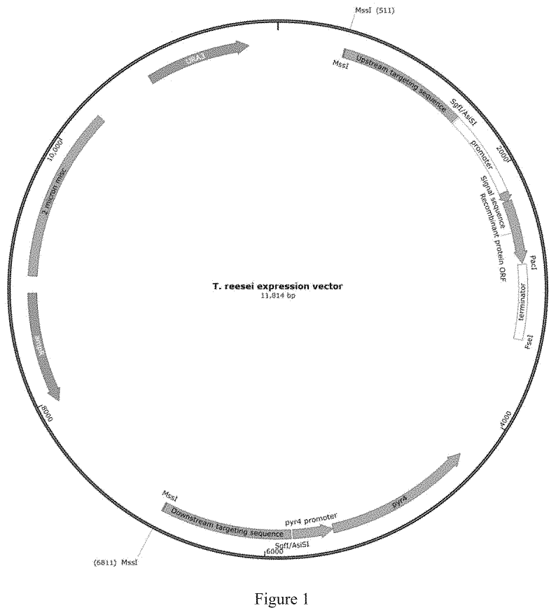

For recombinant protein expression in Trichoderma reesei, the recombinant vector shown in FIG. 1 is constructed using genetic engineering methods known in the art. The recombinant vector comprises an expression construct comprising a protein coding sequence encoding β-lactoglobulin protein of Bos taurus (amino acids 17 to 178 of UniProt sequence P02754; SEQ ID NO: 1 or 2), Ovis aries musimon (UniProt sequence P67975; SEQ ID NO: 4), Ovis aries (UniProt sequence P67976; SEQ ID NO: 5), Equus caballus (amino acids 19-180 of UniProt sequence P02758; SEQ ID NO: 6), Equus asinus (UniProt sequence P13613; SEQ ID NO: 7), Equus caballus (amino acids 19 to 181 of UniProt sequence P07380; SEQ ID NO: 8), Equus asinus (UniProt sequence P19647; SEQ ID NO: 9), or Capra hircus (Uni Prot sequence P02756; SEQ ID NO: 10); codon-optimized for expression in Trichoderma reesei; operably linked to an N-terminal secretion signal (e.g., cbh1, xyn1); and under control of a suitable promoter (e.g., cbh1, xyn1, tef1) and a suitable terminator (e.g., pdc1). The recombinant vector further comprises a polynucleotide that can direct integration of the expression construct at the egl1 locus in the genome of a Trichoderma reesei host cell, selection markers for selection of bacterial and/or fungal transformants, and a bacterial origin of replication. The bacterial selection markers and origin of replication are removed from the recombinant vector via restriction enzyme digestion prior to transformation of the recombinant vector into the host cell.

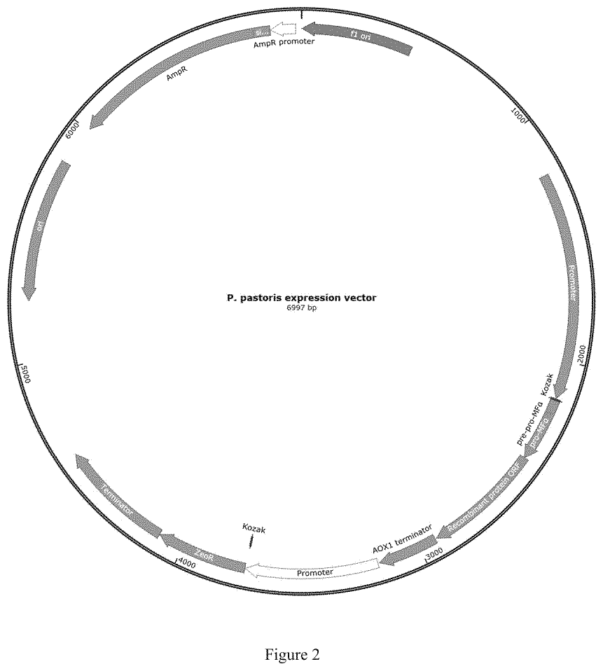

For recombinant protein expression in Pichia pastoris (Komagataella phaffii), the recombinant vector shown in FIG. 2 is constructed using genetic engineering methods known in the art. The recombinant vector comprises an expression construct comprising a protein coding sequence encoding Bos taurus β-lactoglobulin protein (UniProt # P02754 amino acids 17 to 178; SEQ ID NO: 1 or 2), Ovis aries musimon (UniProt sequence P67975; SEQ ID NO: 4), Ovis aries (UniProt sequence P67976; SEQ ID NO: 5), Equus caballus (amino acids 19-180 of UniProt sequence P02758; SEQ ID NO: 6), Equus asinus (UniProt sequence P13613; SEQ ID NO: 7), Equus caballus (amino acids 19 to 181 of UniProt sequence P07380; SEQ ID NO: 8), Equus asinus (UniProt sequence P19647; SEQ ID NO: 9), or Capra hircus (UniProt sequence P02756; SEQ ID NO: 10); codon-optimized for expression in Pichia pastoris (Komagataella phaffii); operably linked to an N-terminal secretion signal (e.g., pre or pre-pro signal peptide of the alpha mating factor of Saccharomyces cerevisiae), and under control of a promoter (e.g., pGAP, pAOX1) and a terminator (e.g., tAOX1 pA signal). The recombinant vector further comprises selection markers for selection of bacterial and/or fungal transformants, and a bacterial origin of replication.

Using QuikChange® Site-Directed Mutagenesis Kit (Agilent, Santa Clara, Calif., USA) and QuikChange Multi Site-Directed Mutagenesis Kit (Agilent, Santa Clara, Calif., USA), site-directed mutagenesis is performed according to the manufacturer's direction on the recombinant vectors above to introduce the amino acid substitutions listed in Table 1 into the encoded β-lactoglobulin protein, as well as any combination of two or more of such amino acid substitutions.

TABLE 1
Amino acid substitution introduced β-lactoglobulin protein encoded by protein coding sequence of recombinant vector.

| | | Introduced | |
|---|---|---|---|
| | | Protease Recognition | |
| Amino Acid Substitution | SEQ ID NO: | or Cleavage Site | Eliminated PTM Site |
| T6S | 1, 2, 4, 5, 6, 7, 8, 9, 10 | | phosphorylation |
| I12L | 1, 2, 4, 5, 10 | Pepsin | |
| T18K or T18R | 1, 2, 4, 5, 10 | Trypsin | O-glycosylation, phosphorylation |
| Y20A, Y20C, Y20D, Y20H, Y20N, | 1, 2, 10 | | phosphorylation |
| Y20S, or Y20T | | | |
| S21D | 1, 2, 4, 5, 6, 7, 8, 9, 10 | | phosphorylation |
| I29L | 1, 2, 4, 5, 6, 7, 8, 9, 10 | Pepsin | phosphorylation |
| S30D | 1, 2, 4, 5, 6, 7, 8, 9, 10 | | phosphorylation |
| S30K | 1, 2, 4, 5, 6, 7, 8, 9, 10 | Trypsin | O-glycosylation, phosphorylation |
| S36Y | 1, 2, 4, 5, 6, 8, 9, 10 | Pepsin and Chymotrypsin | O-glycosylation, phosphorylation |
| S36L | 1, 2, 4, 5, 6, 8, 9, 10 | Pepsin | O-glycosylation, phosphorylation |
| S36K | 1, 2, 4, 5, 6, 8, 9, 10 | Trypsin | O-glycosylation, phosphorylation |
| S36M or S36T | 1, 2, 4, 5, 6, 8, 9, 10 | | phosphorylation |
| K47L | 1, 2, 4, 5, 10 | Pepsin | methylation |
| R47L | 6, 7, 8, 9 | Pepsin | |
| K47P | 1, 2, 4, 5, 10 | Pepsin | methylation |
| R47P | 6, 7, 8, 9 | Pepsin | |
| K47L, G52D, and D53N (converting | 1, 2 | Pepsin | methylation |
| amino acid sequence KPTPEGD at | | | |
| amino acid position 47 through 53 | | | |
| to amino acid sequence LPTPEDN) | | | |
| K47L and G52D (converting amino | 4, 5, 10 | Pepsin | methylation |
| acid sequence KPTPEG at amino | | | |
| acid position 47 through 52 to amino | | | |
| acid sequence LPTPED) | | | |
| R47L and G52D (converting amino | 8, 9 | Pepsin | |
| acid sequence RPTPEG at amino | | | |
| acid position 47 through 52 to amino | | | |
| acid sequence LPTPED) | | | |
| T49K | 1, 2, 4, 5, 6, 7, 8, 9, 10 | Trypsin | O-glycosylation, phosphorylation |
| Q59R | 1, 2, 4, 5, 10 | Trypsin | |
| I72L | 1, 2, 4, 5, 10 | Pepsin | |
| I72F or I72W or I72Y | 1, 2, 4, 5, 10 | Pepsin and chymotrypsin | |
| I72L, A73G, K77E, I78N, A80K, | 1, 2, 4, 5, 10 | Pepsin and trypsin | methylation |
| and V81K (converting amino acid | | | |
| sequence IAEKTKIPAV at amino | | | |
| acid position 72 through 81 to amino | | | |
| acid sequence LGEKIENPKK) | | | |
| F72L | 6, 7 | Pepsin | |
| F72W or F72Y | 6, 7 | Pepsin and chymotrypsin | |
| V72L | 8, 9 | Pepsin | |
| V72F or V72W or V72Y | 8, 9 | Pepsin and chymotrypsin | |
| F72L, A73G, S78N, A80K, and | 6, 7 | Pepsin and trypsin | |
| E81K (converting amino acid | | | |
| sequence FAEKTESPAE at amino | | | |
| acid position 72 through 81 to amino | | | |
| acid sequence LGEKTENPKK) | | | |
| V72L, A73G, Q74E, D78N, A80K, | 8, 9 | Pepsin and trypsin | |
| and V81K (converting amino acid | | | |
| sequence VAQKTEDPAV at amino | | | |
| acid position 72 through 81 to amino | | | |
| acid sequence LGEKTENPKK) | | | |
| K77H | 1, 2, 4, 5, 10 | | methylation |
| I78L | 1, 2, 4, 5, 10 | Pepsin | |
| I78Y or I78W or I78F | 1, 2, 4, 5, 10 | Pepsin and chymotrypsin | |
| S78L | 6, 7 | Pepsin | |
| S78Y or S78W or S78F | 6. 7 | Pepsin and chymotrypsin | |
| D78L | 8, 9 | Pepsin | |
| D78Y or D78W or D78F | 8, 9 | Pepsin and chymotrypsin | |
| A86Y | 1, 2, 4, 5, 10 | Pepsin and chymotrypsin | |
| K83T, 184V, D85N, A86Y, L87Q, | 1, 2, 4, 5, 10 | Pepsin, chymotrypsin, | methylation |
| N88G, and N90R (converting amino | | trypsin | |
| acid sequence KIDALNEN at amino | | | |
| acid position 83 through 90 to amino | | | |
| acid sequence TVNYQGER) | | | |
| D85N, A86Y, N88D, and N90D | 1, 2, 4, 5, 10 | Pepsin and chymotrypsin | |
| (converting amino acid sequence | | | |
| DALNEN at amino acid position 85 | | | |
| through 90 to amino acid sequence | | | |
| NYLDED) | | | |
| N90R | 1, 2, 4, 5, 10 | Trypsin | |
| D90R | 6, 7 | Trypsin | |
| T97S | 1, 2, 4, 5, 6, 8, 9, 10 | | phosphorylation |
| Y102F | 1, 2, 4, 5, 6, 7, 8, 9, 10 | Pepsin and chymotrypsin | phosphorylation |
| S110P or S110L | 1, 2, 4, 5, 10 | Pepsin | O-glycosylation, phosphorylation |
| S110Y or S110W or S110F | 1, 2, 4, 5, 10 | Pepsin and chymotrypsin | O-glycosylation, phosphorylation |
| S110K | 1, 2, 4, 5, 10 | Trypsin | O-glycosylation, phosphorylation |
| A111P or A111L | 1, 2, 4, 5, 6, 7, 10 | Pepsin | |
| A111K | 1, 2, 4, 5, 6, 7, 10 | Trypsin | |
| A111Y or A111W or A111F | 1, 2, 4, 5, 6, 7, 10 | Pepsin and chymotrypsin | |
| E108G, N109P, S110P, A111L, | 1 | Pepsin | O-glycosylation, phosphorylation |
| E112P, P113S, E114A, Q115E, | | | |
| S116HG, and L117M (converting | | | |
| amino acid sequence ENSAEPEQSL | | | |
| at amino acid position 108 through | | | |
| 117 to amino acid sequence | | | |
| GPPLPSAEHGM) | | | |
| E108G, N109P, S110P, A111L, | 2, 4, 5, 10 | Pepsin | O-glycosylation, phosphorylation |
| E112P, P113S, E114A, Q115E, | | | |
| S116HG, L117M, and A118V | | | |
| (converting amino acid sequence | | | |
| ENSAEPEQSLA at amino acid | | | |
| position 108 through 118 to amino | | | |
| acid sequence GPPLPSAEHGMV) | | | |
| K108G, N109P, A110P, A111L, | 6, 7 | Pepsin | O-glycosylation, phosphorylation |
| T112P, P113S, G114A, Q115E, | | | |
| S116HG, and L117M (converting | | | |
| amino acid sequence KNAATPGQSL | | | |
| at amino acid position 108 through | | | |
| 117 to amino acid sequence | | | |
| GPPLPSAEHGM) | | | |
| T125K | 1, 2, 4, 5, 6, 7, 10 | Trypsin | O-glycosylation, phosphorylation |
| T125F or T125W or T125Y | 1, 2, 4, 5, 6, 7, 10 | Pepsin and chymotrypsin | O-glycosylation, phosphorylation |
| T126K | 8, 9 | Trypsin | |
| T126F or T126W or T126Y | 8, 9 | Pepsin and chymotrypsin | |
| E127K | 1.2, 4, 5, 10 | Trypsin | |
| E127L | 1, 2, 4, 5, 10 | Pepsin | |
| E127W or E127F or E127Y | 1, 2, 4, 5, 10 | Pepsin and chymotrypsin | |
| E127K and D130K (converting amino | 1, 2 | Trypsin | |
| acid sequence EVDD at amino acid | | | |
| position 127 through 130 to amino | | | |
| acid sequence KVDK) | | | |
| E127K and N130K (converting amino | 4, 5 | Trypsin | |
| acid sequence EVDN at amino acid | | | |
| position 127 through 130 to amino | | | |
| acid sequence KVDK) | | | |
| V128L | 1, 2, 4, 5, 6, 7, 10 | Pepsin | |
| V129L | 8, 9 | Pepsin | |
| D137R | 1, 2, 4, 5, 10 | Trypsin | |
| S138R | 8, 9 | Trypsin | |
| A142P or A142L | 1, 2, 4, 5, 10 | Pepsin | |
| K141Q, A142P, M145G, and | 1, 2, 4, 5, 10 | Pepsin and trypsin | methylation |
| H146R (converting amino acid | | | |
| sequence KALPMH at amino acid | | | |
| position 141 through 146 to | | | |
| amino acid sequence QPLPGR) | | | |
| H146R | 1, 2, 4, 5, 10 | Trypsin | |
| H147R | 9 | Trypsin | |
| P153L and Q155R (converting amino | 1, 2, 4, 5, 10 | Pepsin and trypsin | |
| acid sequence PTQ at amino acid | | | |
| position 153 through 155 to amino | | | |
| acid sequence LTR) | | | |
| P154L, S155T, and G156R (converting | 8, 9 | Pepsin and trypsin | |
| amino acid sequence PSG at amino | | | |
| acid position 154 through 156 to | | | |
| amino acid sequence LTR) | | | |
| T154K | 1, 2, 4, 5, 6, 7, 10 | Trypsin | O-glycosylation, phosphorylation |
| T154Y or T154W or T154F | 1, 2, 4, 5, 6, 7, 10 | Pepsin and chymotrypsin | O-glycosylation, phosphorylation |
| T154L | 1, 2, 4, 5, 6, 7, 10 | Pepsin | O-glycosylation, phosphorylation |
| F151L, N152D, P153L, T154K, and | 1, 2, 4, 5, 10 | Pepsin and trypsin | O-glycosylation, phosphorylation |
| L156M (converting amino acid | | | |
| sequence FNPTQL at amino acid | | | |
| position 151 through 156 to | | | |
| amino acid sequence LDLKQM) | | | |
| P151L, T154K, and R155Q | 6, 7 | Pepsin and trypsin | O-glycosylation, phosphorylation |
| (converting amino acid sequence | | | |
| PDLTR at amino acid position 151 | | | |
| through 155 to amino acid sequence | | | |
| LDLKQ) | | | |
| Q152L, P154L, S155K, G156Q, and | 8, 9 | Pepsin and trypsin | O-glycosylation, phosphorylation |
| G157M (converting amino acid | | | |
| sequence QDPSGG at amino acid | | | |
| position 152 through 157 to | | | |
| amino acid sequence LDLKQM) | | | |
| Q159R | 1, 4, 5, 10 | Trypsin | |

The recombinant vectors are transformed into Trichoderma reesei or Pichia pastoris (Komagataella phaffii; e.g., strain BG12 [(Biogrammatics, Carlsbad, Calif.]) host cells, and transformants are selected by growth on minimal media or antibiotics for positive selection. The transformants are grown in expression media in 24-well plates, and supernatants are harvested for further analysis. Recombinant host cells that comprise an integrated copy of the expression construct and that secrete a recombinant β-lactoglobulin protein are identified by SDS-PAGE gel analyses of fermentation broth samples.

Recombinant Trichoderma reesei host cells are grown in minimal basal media comprising inorganic salts as sources of phosphate, ammonium, magnesium, potassium, sodium, sulfate, chloride, calcium, iron, manganese, zinc, molybdenum, copper, cobalt, and borate, with a carbohydrate-based carbon source as a starting feedstock, in a stirred fermentation vessel controlled at temperatures of between 25° C., and 34° C. aeration of between 0.2 vvm and 1 vvm, and minimum agitation to ensure proper mixing and dispersion of biomass and nutrients (including oxygen, as delivered in compressed air). The pH of the fermentation broth is controlled at various setpoints ranging from 3.0 and 5.5 with on-demand addition of ammonium hydroxide. Once batch carbohydrate is depleted, a solution containing glucose or lactose is added at specific feed rates ranging from 0.01 through 0.1 g dry substrate per gram dry cell mass (DCM) per hour. The oxygen demand of the culture is satisfied by controlling agitation rate to maintain a target dissolved oxygen set point of between 5% and 50%. When agitation is no longer able to maintain the target dissolved oxygen set-point, aeration is increased up to 2.0 vvm. Once the culture is running at maximum agitation and aeration, the dissolved oxygen is allowed to drop below setpoint. Various antifoams, including but not limited to ACP 1500, Antifoam 204 (Sigma, Sigma-Aldrich Chemical, St. Louis, Mo.), Erol DF6000K, Hodag K-60K, Industrol DF204 (BASF, Florham Park, N.J.), P-2000E (Dow, Midland, Mich.), SAG 471, SAG 5693, SAG 710, SAG 730, Silicone Antifoam (Sigma), Struktol J647, Struktol J673A, and sunflower oil are added as needed to control foaming. Fermentations are harvested after at least 120 h, at biomass concentrations of between 20 g and 50 g dry cell weight (DCW) per L. Biomass is removed from the broth by centrifugation at 5,000×g, and the supernatant is subjected to purification as described below.

Recombinant Pichia pastoris (Komagataella phaffii) host cells are grown in minimal basal media comprising phosphate and nitrogen salts with 80% of glycerol as a starting feedstock, in a stirred fermentation vessel controlled at a temperature of 30° C., aeration of 1 vvm, and minimum agitation of 100 rpm. The pH of the fermentation is controlled at 5 with on-demand addition of ammonium hydroxide. Once batch glycerol is depleted, glycerol is added via a programmed feed recipe at a rate of 6 g/L/h. The oxygen demand of the strain is satisfied by controlling agitation rate. When agitation is no longer able to maintain the dissolved oxygen set-point, 100% oxygen gas is sparged into the vessel to control dissolved oxygen. The pH of fermentation is shifted from 5 to 3 once batched glycerol is depleted. Antifoam C is added as needed to control foam. The fermentations are harvested after at least 100 hours, at cell densities of 600-800 at OD600. Biomass is removed from the broth by centrifugation at 5,000×g, and the supernatant is subjected to purification as described below.

The supernatant is concentrated over 100 kDa molecular weight cut-off (MWCO) membranes. The concentrate retentate is diafiltered over 5 kda MWCO membranes into 50 mM Imidazole, pH 6.8. The concentrated retentate is passed over a Q sepharose FF column. The mobile phase is 50 mM Imidazole, pH 6.8, and the recombinant β-lactoglobulin protein is eluted on a 2M NaCl gradient. The gradient is run from 0-30% over 30 column volumes. Peak fractions are collected and analyzed on RP-HPLC. Peaks containing recombinant β-lactoglobulin protein with a purity of greater than 85% are pooled for final diafiltration into water.

### Example 2: Analysis of Protease Digestion of Recombinant D-Lactoglobulin Protein

The purified recombinant β-lactoglobulin proteins of Example 1 are subjected to protease digestion at 37° C. for 0.5 hr, 1 hr, 4 hr, 6 hr, or overnight with pepsin from porcine gastric mucosa, trypsin from human pancreas, or chymotrypsin from human pancreas, all obtained from Sigma (Sigma-Aldrich Chemicals, St. Louis, Mo.). Pepsin digestion is conducted at pH 1.5 or 3, and trypsin and chymotrypsin digestions are conducted at pH 6, 7, or 8. The protease digestion procedures are conducted according to experimental details described in Pena-Ramos & Xiong 2001 J. Dairy Sci. 84:2577-2583; Quintieri et al. 2017 J Food Sci Technol 54:1910-1916; Selo et al. 1999 Clinical and Experimental Allergy 29:1055-1063; Morisawa et al, 200) Clinical & Experimental Allergy 39: 918-925; or Kondo et al. 2007 Allergy Asthma Clin Immunol, 3(1):1-9.

The purified recombinant β-lactoglobulin proteins of Example 1 are also subjected to a simulated in vitro digestion system as described in Wróblewska et al. 2016 Food Research International 83:95-101, Bossios et al. 2011 Clinical and Translational Allergy 1:6, or Benedé et al. 2014 Food Research International 62:1127-1133.

The identity of hydrolyzed peptides is determined as described in Quintieri et al. 2017 J Food Sci Technol 54:1910-1916, Selo et al. 1999 Clinical and Experimental Allergy 29:1055-1063; or Kondo et al. 2007 Allergy Asthma Clin Immunol 3(0):1-9.

### Example 3: Allergenicity Testing of Recombinant β-Lactoglobulin Protein

Potential allergenicity (i.e., recognition by sera from allergic humans and IgE binding) of the purified recombinant β-lactoglobulin proteins of Example 1, as well as of their protease-hydrolyzed peptides of Example 2, can be predicted or measured by a variety of methods known in the art.

Predictions of allergenicity can be based on global sequence comparisons with known allergens, and/or on sequence comparisons of contiguous amino acid windows of different lengths (e.g., 80, 70, 60, 50, 40, 30, 20, 10, 8, or 6 amino acids) with known allergenic peptides. Skilled artisans are able to identify and use a suitable database of known allergenic epitopes and suitable sequence comparison algorithms for this purpose.

Allergenicity can also be predicted based on homology to proteins of human origin, wherein a greater degree of homology suggests a lower likelihood of allergenicity.

Computational tools for predicting allergenicity are known in the art (see, for example, Bui et al. 2005. Immunogenetics 57:304-314; Peters & Sette. 2005. BMC Bioinformatics 6:132; Karosiene et al. 2012. Immunogenetics 64(3):177-186; Zhang et al. 2009. Bioinformatics 25(10):1293-1299; Nielsen et al. 2007. BMC Bioinformatics 8:238; Stumiolo et al. 1999. Nat Biotechnol 17:555-561; Tenzer et al. 2005. Cell Mol Life Sci 62:1025-1037; Peters et al. 2003. J Immunol 171:1741-1749; Nielsen et al. 2005. Immunogenetics 57:33-41; Kesmir et al. 2002. Protein Eng 15:287-296; Larsen 2005. Eur J Immunol 35:2295-2303; Nielsen et al. 2003. Protein Sci 12:1007-1017; Buus et al. 2003. Tissue Antigens 62:378-384; Peters et al. 2003. J Immunol 171:1741-1749; Stranzl et al. 2010. Immunogenetics 62:357-68; Chou & Fasman. 1978. Adv Enzymol Relat Areas Mol Biol 47:45-148; Emini et al. 1985. J Virol 55:836-839; Karplus & Schulz. 1985. Naturwissenschaften 72:212-213; Kolaskar & Tongaonkar. 1990. FEBS Lett 276:172-174: Parker et al. 1986. Biochemistry 25:5425-5432; Larsen et al. 2006. Immunome Res 2:2; Ponomarenko & Bourne. 2007. BMC Struct Biol 764; Haste et al. 2006. Protein Sci 15:2558-2567; Ponomarenko et al. 2008. BMC Bioinformatics 9:514; Bui et al. 2006. BMC Bioinformatics 7:153; Bui et al. 2007. BMC Bioinformatics 8:361; Beaver et al. 2007. Immunome Res 3:3), and are publicly accessible, for example, at http://tools.iedb.org/main/.

Allergenicity can also be predicted using experimental methods. For example, allergenicity can be predicted using a T-cell proliferation assay (e.g., using human peripheral blood mononuclear cells), an IgE binding assay (e.g., using an ELISA or competitive ELISA on immobilized milk protein or milk protein fragments), epitope mapping using phage display, and using direct and competitive inhibition enzyme immunoassays (see, for example, Selo et al. 1999 Clinical and Experimental Allergy 29:1055-1063, Benede et al. 2014 Food Research International 62:1127-1133, Quintieri et al. 2017 J Food Sci Technol 54:1910-1916, Cocco et al. 2007 Clinical and Experimental Allergy 37:831-838; or Wróblewska et al. 2016 Food Research International 83:95-101).

Allergenicity can also be measured using skin prick tests, blood tests, oral food challenges, and population studies.

Results are compared to those of the corresponding native β-lactoglobulin protein or protease-hydrolyzed peptides thereof.

1-128. (canceled)

129. A composition comprising or consisting of a milk protein component, wherein the milk protein component comprises or consists of a recombinant milk protein comprising a non-native protease site in a solvent-exposed region of a corresponding native milk protein, wherein the non-native protease site attenuates or essentially eliminates allergenicity of the recombinant milk protein compared to the corresponding native milk protein, and wherein the recombinant milk protein retains one or more functional attributes of the corresponding native milk protein.

130. The composition of claim 129, wherein the recombinant milk protein is a recombinant β-lactoglobulin protein.

131. The composition of claim 130, wherein the recombinant β-lactoglobulin protein is produced in a recombinant fungal host cell.

132. The composition of claim 131, wherein the recombinant fungal host cell is a recombinant filamentous fungal host cell.

133. The composition of claim 132, wherein the recombinant filamentous fungal hostcell is derived from a member of a genera selected from the group consisting of Aspergillus, Fusarium, Myceliophthora, and Trichoderma.

134. The composition of claim 133, wherein the recombinant β-lactoglobulin protein comprises an amino acid sequence that is at least 40% identical to SEQ ID NO: 2.

135. The composition of claim 134, wherein the non-native protease site comprises a single amino acid substitution selected from the group consisting of: I12L, T18K, T18R, I29L, S30K, S36Y, S36L, S36K, K47L, K47P, T49K, Q59R, I72L, I72F, I72W, I72Y, I78L, I78Y, I78W, I78F, A86Y, N90R, Y102F, S110P, S110L, S110Y, S110W, S110F, S110K, A111P, A111K, A111L, A111Y, A111W, A111F, T125K, T125F, T125W, T125Y, E127K, E127L, E127W, E127F, E127Y, V128L, D137R, A142P, A142L, H146R, T154K, T154Y, T154L, T154W, T154F, Q159R, E108G, N109P, E112P, P113S, E114A, Q115E, A116HG, L117M, and A118V of SEQ ID NO: 2.

136. The composition of claim 134, wherein the non-native protease site comprises two or more amino acid substitutions selected from the group consisting of: I12L, T18K, T18R, I29L, S30K, S36Y, S36L, S36K, K47L, K47P, T49K, Q59R, I72L, I72F, I72W, I72Y, I78L, I78Y, I78W, I78F, A86Y, N90R, Y102F, S110P, S110L, S110Y, S110W, S110F, S110K, A111P, A111K, A111L, A111Y, A111W, A111F, T125K, T125F, T125W, T125Y, E127K, E127L, E127W, E127F, E127Y, V128L, D137R, A142P, A142L, H146R, T154K, T154Y, T154L, T154W, T154F, and Q159R of SEQ ID NO: 2.

137. The composition of claim 134, wherein the non-native protease site comprises amino acid substitutions K47L, G52D, and D53N of SEQ ID NO: 2.

138. The composition of claim 134, wherein the non-native protease site comprises amino acid substitutions I72L, A73G, K77E, I78N, A80K, and V81K of SEQ ID NO: 2.

139. The composition of claim 134, wherein the non-native protease site comprises amino acid substitutions K83T, I84V, D85N, A86Y, L87Q, N88G, and N90R of SEQ ID NO: 2.

140. The composition of claim 134, wherein the non-native protease site comprises amino acid substitutions D85N, A86Y, N88D, and N90D of SEQ ID NO: 2.

141. The composition of claim 134, wherein the modification comprises or consists of amino acid substitutions E108G, N109P, S110P, A111L, E112P, P113S, E114A, Q115E, S116HG, L117M, and A118V of SEQ ID NO: 2.

142. The composition of claim 134, wherein the non-native protease site comprises amino acid substitutions E127K and D130K of SEQ ID NO: 2.

143. The composition of claim 134, wherein the non-native protease site comprises amino acid substitutions K141Q, A142P, M145G, and H146R of SEQ ID NO: 2.

144. The composition of claim 134, wherein the non-native protease site comprises amino acid substitutions P153L and Q155R of SEQ ID NO: 2.

145. The composition of claim 134, wherein the non-native protease site comprises amino acid substitutions F151L, N152D, P153L, T154K, and L156M of SEQ ID NO: 2.

146. The composition of claim 135, wherein the composition has an allergenicity of no more than 90% relative to that of a comparable composition.

147. The composition of claim 135, wherein the composition has an allergenicity of no more than 80% relative to that of a comparable composition.

148. The composition of claim 135, wherein the composition has an allergenicity of no more than 70% relative to that of a comparable composition.

149. The composition of claim 135, wherein the composition has an allergenicity of no more than 60% relative to that of a comparable composition.

150. The composition of claim 135, wherein the composition has an allergenicity of no more than 50% relative to that of a comparable composition.

151. The composition of claim 135, wherein the composition consists of the milk protein component.

152. The composition of claim 151, wherein the milk protein component consists of the recombinant milk protein.

153. The composition of claim 151, wherein the composition is a powder.

154. The composition of claim 151, wherein the composition is a food product.

155. The composition of claim 154, wherein the food product is a dairy product or supplemented dairy product.

156. The composition of claim 155, wherein the dairy product or supplemented dairy product is selected from the group consisting of milk, yogurt, cheese, dairy-based sauce, dairy spread, cream, frozen confection, dairy dessert, butter, dairy powder, infant formula, milk protein concentrate, milk protein isolate, nutritional supplement, texturizing blend, flavoring blend, coloring blend, pudding, and fermented dairy product.

157. The composition of claim 154, wherein the food product is an animal meat, animal meat product, or supplemented animal meat product.

158. The composition of claim 154, wherein the food product is an egg, egg product, or supplemented egg product.

159. The composition of claim 135, wherein the composition comprises a polymer or polymer network comprising linked repeated protein monomers, wherein the repeated protein monomers comprise the recombinant milk protein.


SEQUENCE LISTING (10 sequences)

SEQ ID NO: 1 - protein, 162 aa, Bos taurus
  misc_feature: B-lactoglobulin isoform A
  VARIANT (1)..(1): Leu, Ala, Gly, Ile or Val
  VARIANT (2)..(2): Ile, Ala, Gly, Leu or Val
  VARIANT (3)..(3): Val, Ala, Gly, Ile or Leu
  VARIANT (4)..(4): Thr, Met or Ser
  VARIANT (5)..(5): Gln, Asp, Glu or Asn
  VARIANT (6)..(6): Thr, Met or Ser
  VARIANT (7)..(7): Met, Ser or Thr
  VARIANT (8)..(8): Lys, His or Arg
  VARIANT (9)..(9): Gly, Ala, Ile, Leu or Val
  VARIANT (10)..(10): Leu, Ala, Gly, Ile or Val
  VARIANT (11)..(11): Asp, Glu, Asn or Gln
  VARIANT (12)..(12): Ile, Leu, Ala, Gly or Val
  VARIANT (13)..(13): Gln, Asp, Glu or Asn
  VARIANT (15)..(15): Val, Ala, Gly, Ile or Leu
  VARIANT (16)..(16): Ala, Gly, Ile, Leu or Val
  VARIANT (18)..(18): Thr, Lys or Arg
  VARIANT (19)..(19): Trp, Cys, Asp, Glu, Phe, Gly, His, Lys, Asn, Pro, Gln, Arg, Ser, Thr or Tyr
  VARIANT (20)..(20): Tyr, Ala, Cys, Asp, Glu, Gly, His, Met, Asn, Gln, Arg, Ser, Thr, or Val
  VARIANT (21)..(21): Ser, Asp or Gly
  VARIANT (22)..(22): Leu, Cys, Asp, Glu, Gly, His, Lys, Asn, Pro, Gln, Arg, Ser, Thr or Trp
  VARIANT (23)..(23): Ala, Cys, Gly, His, Lys, Pro or Trp
  VARIANT (24)..(24): Met, Ala, Cys, Asp, Glu, Gly, Asn, Pro, Gln or Ser
  VARIANT (25)..(25): Ala, Cys, Asp, Glu, Gly, Pro or Trp
  VARIANT (26)..(26): Ala, Cys, Asp, Glu, Gly, Pro or Trp
  VARIANT (27)..(27): Ser, Cys, Asp or Gly
  VARIANT (29)..(29): Ile, Leu, Cys, Asp, Glu, Gly, His, Lys, Asn, Pro or Arg
  VARIANT (30)..(30): Ser, Lys, Cys, Asp or Gly
  VARIANT (31)..(31): Leu, Ala, Cys, Asp, Glu, Gly, His, Ile, Lys, Met, Asn, Pro, Gln, Arg, Ser, Thr, Val or Trp
  VARIANT (32)..(32): Leu, Ala, Cys, Asp, Glu, Gly, His, Ile, Lys, Met, Asn, Pro, Gln, Arg, Thr, Val or Trp
  VARIANT (33)..(33): Asp, Glu, Asn or Gln
  VARIANT (34)..(34): Ala, Gly, Ile, Leu or Val
  VARIANT (35)..(35): Gln, Asp, Glu or Asn
  VARIANT (36)..(36): Ser, Tyr, Leu, Lys, Met or Thr
  VARIANT (37)..(37): Ala, Gly, Ile, Leu, or Val
  VARIANT (39)..(39): Leu, Ala, Gly, Ile or Val
  VARIANT (40)..(40): Arg or His
  VARIANT (41)..(41): Val, Arg, Gly, Ile or Leu
  VARIANT (42)..(42): Tyr or Gly
  VARIANT (43)..(43): Val, Ala, Gly, Ile or Leu
  VARIANT (44)..(44): Glu, Asp, Gly, Asn or Gln
  VARIANT (45)..(45): Glu, Asp, Asn or Gln
  VARIANT (46)..(46): Leu, Ala, Gly, Ile or Val
  VARIANT (47)..(47): Lys, Leu, Pro, His or Arg
  VARIANT (47)..(53): LysProThrProGluGlyAsp or LeuProThrProGluAspAsn
  VARIANT (48)..(48): Pro or Gly
  VARIANT (49)..(49): Thr, Lys, Gly, Met or Ser
  VARIANT (50)..(50): Pro or Gly
  VARIANT (51)..(51): Glu, Asp, Gly, Asn or Gln
  VARIANT (52)..(52): Gly, Ala, Ile, Leu or Val
  VARIANT (53)..(53): Asp, Glu, Asn or Gln
  VARIANT (54)..(54): Leu, Ala, Gly, Ile or Val
  VARIANT (55)..(55): Glu, Asp, Asn or Gln
  VARIANT (56)..(56): Ile, Ala, Gly, Leu or Val
  VARIANT (57)..(57): Leu, Ala, Gly, Ile or Val
  VARIANT (58)..(58): Leu, Ala, Gly, Ile or Val
  VARIANT (59)..(59): Gln, Arg, Asp, Glu or Asn
  VARIANT (61)..(61): Trp, Phe or Tyr
  VARIANT (62)..(62): Glu, Asp, Asn or Gln
  VARIANT (63)..(63): Asn, Asp, Glu or Gln
  VARIANT (64)..(64): Asp, Ala, Ile, Leu or Val
  VARIANT (65)..(65): Glu, Asp, Asn or Gln
  VARIANT (67)..(67): Ala, Gly, Ile, Leu or Val
  VARIANT (68)..(68): Gln, Asp, Glu or Asn
  VARIANT (71)..(71): Ile, Ala, Gly, Leu or Val
  VARIANT (72)..(72): Ile, Leu, Phe, Trp, Tyr, Ala, Gly or Val
  VARIANT (72)..(81): IleAlaGluLysThrLysIleProAlaVal or LeuGlyGluLysThrGluAsnProLysLys
  VARIANT (73)..(73): Ala, Gly, Ile, Leu or Val
  VARIANT (74)..(74): Glu, Asp, Asn or Gln
  VARIANT (75)..(75): Lys or His
  VARIANT (76)..(76): Thr, Met or Ser
  VARIANT (77)..(77): Lys or His
  VARIANT (78)..(78): Ile, Leu, Tyr, Trp, Phe, Ala, Gly or Val
  VARIANT (80)..(80): Ala, Gly, Ile, Leu or Val
  VARIANT (81)..(81): Val, Ala, Gly, Ile or Leu
  VARIANT (82)..(82): Phe, Trp or Tyr
  VARIANT (83)..(83): Lys or His
  VARIANT (83)..(90): LysIleAspAlaLeuAsnGluAsn or ThrValAsnTyrGlnGlyGluArg
  VARIANT (84)..(84): Ile, Ala, Gly, Leu or Val
  VARIANT (85)..(85): Asp, Glu, Asn or Gln
  VARIANT (85)..(90): AspAlaLeuAsnGluAsn or AsnTyrLeuAspGluAsp
  VARIANT (86)..(86): Ala, Tyr, Gly, Ile, Leu, Gln or Val
  VARIANT (90)..(90): Asn or Arg
  VARIANT (97)..(97): Thr, Met or Ser
  VARIANT (98)..(98): Asp, Glu, Asn or Gln
  VARIANT (100)..(100): Lys or His
  VARIANT (101)..(101): Lys or His
  VARIANT (102)..(102): Tyr or Phe
  VARIANT (103)..(103): Leu, Ala, Gly, Ile or Val
  VARIANT (104)..(104): Leu, Ala, Gly, Ile or Val
  VARIANT (105)..(105): Phe, Trp or Tyr
  VARIANT (107)..(107): Met, Ser or Thr
  VARIANT (108)..(108): Glu, Asp, Asn or Gln
  VARIANT (108)..(117): GluAsnSerAlaGluProGluGlnSerLeu or GlyProProLeuProSerAlaGluHisGlyMet (wherein X116HisGly increases the protein length to 163 amino acids)
  VARIANT (109)..(109): Asn, Asp, Glu or Gln
  VARIANT (110)..(110): Ser, Pro, Leu, Tyr, Trp, Phe, Lys, Met or Thr
  VARIANT (111)..(111): Ala, Pro, Lys, Leu, Tyr, Trp, Phe, Gly, Ile or Val
  VARIANT (112)..(112): Glu, Asp, Asn or Gln
  VARIANT (114)..(114): Glu, Asp, Asn or Gln
  VARIANT (115)..(115): Gln, Asp, Glu or Asn
  VARIANT (116)..(116): Ser, Met or Thr
  VARIANT (117)..(117): Leu, Ala, Gly, Ile or Val
  VARIANT (118)..(118): Val, Gly, Ile or Leu
  VARIANT (120)..(120): Gln, Asp, Glu or Asn
  VARIANT (122)..(122): Leu, Ala, Gly, Ile or Val
  VARIANT (123)..(123): Val, Ala, Gly, Ile or Leu
  VARIANT (124)..(124): Arg or His
  VARIANT (125)..(125): Thr, Lys, Phe, Trp, Tyr, Met or Ser
  VARIANT (127)..(127): Glu, Lys, Leu, Trp, Phe, Tyr, Asp, Asn or Gln
  VARIANT (127)..(130): GluValAspAsp or LysValAspLys
  VARIANT (128)..(128): Val, Leu, Ala, Gly or Ile
  VARIANT (129)..(129): Asp, Glu, Asn or Gln
  VARIANT (130)..(130): Asp, Glu, Asn or Gln
  VARIANT (131)..(131): Glu, Asp, Asn or Gln
  VARIANT (132)..(132): Ala, Gly, Ile, Leu or Val
  VARIANT (133)..(133): Leu, Ala, Gly, Ile or Val
  VARIANT (134)..(134): Glu, Asp, Asn or Gln
  VARIANT (136)..(136): Phe, Trp or Tyr
  VARIANT (137)..(137): Asp, Arg, Glu, Asn or Gln
  VARIANT (139)..(139): Ala, Gly, Ile, Leu or Val
  VARIANT (140)..(140): Leu, Ala, Gly, Ile or Val
  VARIANT (141)..(146): LysAlaLeuProMetHis or GlnProLeuProGlyArg
  VARIANT (142)..(142): Ala, Pro, Leu, Gly, Ile, Leu or Val
  VARIANT (143)..(143): Leu, Ala, Gly, Ile or Val
  VARIANT (145)..(145): Met, Ser or Thr
  VARIANT (146)..(146): His, Arg, Lys or Pro
  VARIANT (147)..(147): Ile, Ala, Cys, Asp, Glu, Gly, His, Lys, Leu, Asn, Pro, Gln, Arg, Ser, Thr or Val
  VARIANT (149)..(149): Leu, Ala, Cys, Asp, Glu, Gly, His, Ile, Lys, Asn, Pro, Gln, Arg, Ser, Thr or Val
  VARIANT (150)..(150): Ser, Cys, Asp, Glu, Gly, Met, Asn or Thr
  VARIANT (151)..(151): Phe, Ala, Cys, Asp, Glu, Gly, His, Ile, Lys, Asn, Pro, Gln, Arg, Ser, Thr, Val, Trp or Tyr
  VARIANT (151)..(156): PheAsnProThrGlnLeu or LeuAspLeuLysGlnMet
  VARIANT (152)..(152): Asn, Cys, Asp, Glu, Gly or Gln
  VARIANT (153)..(155): ProThrGln or LeuThrArg
  VARIANT (154)..(154): Thr, Lys, Tyr, Leu, Trp, Phe, Gly, His, Met or Ser
  VARIANT (155)..(155): Gln, Asp, Glu or Asn
  VARIANT (156)..(156): Leu, Ala, Cys, Asp, Glu, Gly, His, Ile, Lys, Met, Asn, Pro, Gln, Arg, Ser, Thr or Val
  VARIANT (157)..(157): Glu, Cys, Asp, Gly, His, Asn, Pro or Gln
  VARIANT (158)..(158): Glu, Asp, Asn or Gln
  VARIANT (159)..(159): Gln, Arg, Asp, Glu or Asn
  VARIANT (161)..(161): His, Lys or Arg
  VARIANT (162)..(162): Ile, Ala, Gly, Leu or Val
XXXXXXXXXXXXXKXXGXXXXXXXXXXDXXXXXXXXXPXXXXXXXXXXXXXXXXXXXXXKXXXXXCXXKKXXXXXXXXPXXXXXXXLNEXKVLVLDXXYXXXXXXCXXXXXXXXXXXXCXCXXXXPXXXXXXXXKXXKXXKXXPXXXRXXXXPXXXXXXCXX

SEQ ID NO: 2 - protein, 162 aa, Bos taurus
  misc_feature: B-lactoglobulin isoform B
  VARIANT (1)..(1): Leu, Ala, Gly, Ile or Val
  VARIANT (2)..(2): Ile, Ala, Gly, Leu or Val
  VARIANT (3)..(3): Val, Ala, Gly, Ile or Leu
  VARIANT (4)..(4): Thr, Met or Ser
  VARIANT (5)..(5): Gln, Asp, Glu or Asn
  VARIANT (6)..(6): Thr, Met or Ser
  VARIANT (7)..(7): Met, Ser or Thr
  VARIANT (8)..(8): Lys, His or Arg
  VARIANT (9)..(9): Gly, Ala, Ile, Leu or Val
  VARIANT (10)..(10): Leu, Ala, Gly, Ile or Val
  VARIANT (11)..(11): Asp, Glu, Asn or Gln
  VARIANT (12)..(12): Ile, Leu, Ala, Gly or Val
  VARIANT (13)..(13): Gln, Asp, Glu or Asn
  VARIANT (15)..(15): Val, Ala, Gly, Ile or Leu
  VARIANT (16)..(16): Ala, Gly, Ile, Leu or Val
  VARIANT (18)..(18): Thr, Lys or Arg
  VARIANT (19)..(19): Trp, Cys, Asp, Glu, Phe, Gly, His, Lys, Asn, Pro, Gln, Arg, Ser, Thr or Tyr
  VARIANT (20)..(20): Tyr, Ala, Cys, Asp, Glu, Gly, His, Met, Asn, Gln, Arg, Ser, Thr, or Val
  VARIANT (21)..(21): Ser, Asp or Gly
  VARIANT (22)..(22): Leu, Cys, Asp, Glu, Gly, His, Lys, Asn, Pro, Gln, Arg, Ser, Thr or Trp
  VARIANT (23)..(23): Ala, Cys, Gly, His, Lys, Pro or Trp
  VARIANT (24)..(24): Met, Ala, Cys, Asp, Glu, Gly, Asn, Pro, Gln or Ser
  VARIANT (25)..(25): Ala, Cys, Asp, Glu, Gly, Pro or Trp
  VARIANT (26)..(26): Ala, Cys, Asp, Glu, Gly, Pro or Trp
  VARIANT (27)..(27): Ser, Cys, Asp or Gly
  VARIANT (29)..(29): Ile, Leu, Cys, Asp, Glu, Gly, His, Lys, Asn, Pro or Arg
  VARIANT (30)..(30): Ser, Lys, Cys, Asp or Gly
  VARIANT (31)..(31): Leu, Ala, Cys, Asp, Glu, Gly, His, Ile, Lys, Met, Asn, Pro, Gln, Arg, Ser, Thr, Val or Tyr
  VARIANT (32)..(32): Leu, Ala, Cys, Asp, Glu, Gly, His, Ile, Lys, Met, Asn, Pro, Gln, Arg, Thr, Val or Trp
  VARIANT (33)..(33): Asp, Glu, Asn or Gln
  VARIANT (34)..(34): Ala, Gly, Ile, Leu or Val
  VARIANT (35)..(35): Gln, Asp, Glu or Asn
  VARIANT (36)..(36): Ser, Tyr, Leu, Lys, Met or Thr
  VARIANT (37)..(37): Ala, Gly, Ile, Leu, or Val
  VARIANT (39)..(39): Leu, Ala, Gly, Ile or Val
  VARIANT (40)..(40): Arg or His
  VARIANT (41)..(41): Val, Ala, Gly, Ile or Leu
  VARIANT (42)..(42): Tyr or Gly
  VARIANT (43)..(43): Val, Ala, Gly, Ile or Leu
  VARIANT (44)..(44): Glu, Asp, Gly, Asn or Gln
  VARIANT (45)..(45): Glu, Asp, Asn or Gln
  VARIANT (46)..(46): Leu, Ala, Gly, Ile or Val
  VARIANT (47)..(47): Lys, Leu, Pro, His or Arg
  VARIANT (47)..(53): LysProThrProGluGlyAsp or LeuProThrProGluAspAsn
  VARIANT (48)..(48): Pro or Gly
  VARIANT (49)..(49): Thr, Lys, Gly, Met or Ser
  VARIANT (50)..(50): Pro or Gly
  VARIANT (51)..(51): Glu, Asp, Gly, Asn or Gln
  VARIANT (52)..(52): Gly, Ala, Ile, Leu or Val
  VARIANT (53)..(53): Asp, Glu, Asn or Gln
  VARIANT (54)..(54): Leu, Ala, Gly, Ile or Val
  VARIANT (55)..(55): Glu, Asp, Asn or Gln
  VARIANT (56)..(56): Ile, Ala, Gly, Leu or Val
  VARIANT (57)..(57): Leu, Ala, Gly, Ile or Val
  VARIANT (58)..(58): Leu, Ala, Gly, Ile or Val
  VARIANT (59)..(59): Gln, Arg, Asp, Glu or Asn
  VARIANT (61)..(61): Trp, Phe or Tyr
  VARIANT (62)..(62): Glu, Asp, Asn or Gln
  VARIANT (63)..(63): Asn, Asp, Glu or Gln
  VARIANT (64)..(64): Gly, Ala, Ile, Leu or Val
  VARIANT (65)..(65): Glu, Asp, Asn or Gln
  VARIANT (67)..(67): Ala, Gly, Ile, Leu or Val
  VARIANT (68)..(68): Gln, Asp, Glu or Asn
  VARIANT (71)..(71): Ile, Ala, Gly, Leu or Val
  VARIANT (72)..(72): Ile, Leu, Phe, Trp, Tyr, Ala, Gly or Val
  VARIANT (72)..(81): IleAlaGluLysThrLysIleProAlaVal or LeuGlyGluLysThrGluAsnProLysLys
  VARIANT (73)..(73): Ala, Gly, Ile, Leu or Val
  VARIANT (74)..(74): Glu, Asp, Asn or Gln
  VARIANT (75)..(75): Lys or His
  VARIANT (76)..(76): Thr, Met or Ser
  VARIANT (77)..(77): Lys or His
  VARIANT (78)..(78): Ile, Leu, Tyr, Trp, Phe, Ala, Gly or Val
  VARIANT (80)..(80): Ala, Gly, Ile, Leu or Val
  VARIANT (81)..(81): Val, Ala, Gly, Ile or Leu
  VARIANT (82)..(82): Phe, Trp or Tyr
  VARIANT (83)..(83): Lys or His
  VARIANT (83)..(90): LysIleAspAlaLeuAsnGluAsn or ThrValAsnTyrGlnGlyGluArg
  VARIANT (84)..(84): Ile, Ala, Gly, Leu or Val
  VARIANT (85)..(85): Asp, Glu, Asn or Gln
  VARIANT (85)..(90): AspAlaLeuAsnGluAsn or AsnTyrLeuAspGluAsp
  VARIANT (86)..(86): Ala, Tyr, Gly, Ile, Leu, Gln or Val
  VARIANT (90)..(90): Asn or Arg
  VARIANT (97)..(97): Thr, Met or Ser
  VARIANT (98)..(98): Asp, Glu, Asn or Gln
  VARIANT (100)..(100): Lys or His
  VARIANT (101)..(101): Lys or His
  VARIANT (102)..(102): Tyr or Phe
  VARIANT (103)..(103): Leu, Ala, Gly, Ile or Val
  VARIANT (104)..(104): Leu, Ala, Gly, Ile or Val
  VARIANT (105)..(105): Phe, Trp or Tyr
  VARIANT (107)..(107): Met, Ser or Thr
  VARIANT (108)..(108): Glu, Asp, Asn or Gln
  VARIANT (108)..(118): GluAsnSerAlaGluProGluGlnSerLeuAla or GlyProProLeuProSerAlaGluHisGlyMetVal (wherein X116HisGly increases the protein length to 163 amino acids)
  VARIANT (109)..(109): Asn, Asp, Glu or Gln
  VARIANT (110)..(110): Ser, Pro, Leu, Tyr, Trp, Phe, Lys, Met or Thr
  VARIANT (111)..(111): Ala, Pro, Lys, Leu, Tyr, Trp, Phe, Gly, Ile or Val
  VARIANT (112)..(112): Glu, Asp, Asn or Gln
  VARIANT (114)..(114): Glu, Asp, Asn or Gln
  VARIANT (115)..(115): Gln, Asp, Glu or Asn
  VARIANT (116)..(116): Ser, Met or Thr
  VARIANT (117)..(117): Leu, Ala, Gly, Ile or Val
  VARIANT (118)..(118): Ala, Gly, Ile, Leu or Val
  VARIANT (120)..(120): Gln, Asp, Glu or Asn
  VARIANT (122)..(122): Leu, Ala, Gly, Ile or Val
  VARIANT (123)..(123): Val, Ala, Gly, Ile or Leu
  VARIANT (124)..(124): Arg or His
  VARIANT (125)..(125): Thr, Lys, Phe, Trp, Tyr, Met or Ser
  VARIANT (127)..(127): Glu, Lys, Leu, Trp, Phe, Tyr, Asp, Asn or Gln
  VARIANT (127)..(130): GluValAspAsp or LysValAspLys
  VARIANT (128)..(128): Val, Leu, Ala, Gly or Ile
  VARIANT (129)..(129): Asp, Glu, Asn or Gln
  VARIANT (130)..(130): Asp, Glu, Asn or Gln
  VARIANT (131)..(131): Glu, Asp, Asn or Gln
  VARIANT (132)..(132): Ala, Gly, Ile, Leu or Val
  VARIANT (133)..(133): Leu, Ala, Gly, Ile or Val
  VARIANT (134)..(134): Glu, Asp, Asn or Gln
  VARIANT (136)..(136): Phe, Trp or Tyr
  VARIANT (137)..(137): Asp, Arg, Glu, Asn or Gln
  VARIANT (139)..(139): Ala, Gly, Ile, Leu or Val
  VARIANT (140)..(140): Leu, Ala, Gly, Ile or Val
  VARIANT (141)..(146): LysAlaLeuProMetHis or GlnProLeuProGlyArg
  VARIANT (142)..(142): Ala, Pro, Leu, Gly, Ile, Leu or Val
  VARIANT (143)..(143): Leu, Ala, Gly, Ile or Val
  VARIANT (145)..(145): Met, Ser or Thr
  VARIANT (146)..(146): His, Arg, Lys or Pro
  VARIANT (147)..(147): Ile, Ala, Cys, Asp, Glu, Gly, His, Lys, Leu, Asn, Pro, Gln, Arg, Ser, Thr or Val
  VARIANT (149)..(149): Leu, Ala, Cys, Asp, Glu, Gly, His, Ile, Lys, Asn, Pro, Gln, Arg, Ser, Thr or Val
  VARIANT (150)..(150): Ser, Cys, Asp, Glu, Gly, Met, Asn or Thr
  VARIANT (151)..(151): Phe, Ala, Cys, Asp, Glu, Gly, His, Ile, Lys, Asn, Pro, Gln, Arg, Ser, Thr, Val, Trp or Tyr
  VARIANT (151)..(156): PheAsnProThrGlnLeu or LeuAspLeuLysGlnMet
  VARIANT (152)..(152): Asn, Cys, Asp, Glu, Gly or Gln
  VARIANT (153)..(155): ProThrGln or LeuThrArg
  VARIANT (154)..(154): Thr, Lys, Tyr, Leu, Trp, Phe, Gly, His, Met or Ser
  VARIANT (155)..(155): Gln, Asp, Glu or Asn
  VARIANT (156)..(156): Leu, Ala, Cys, Asp, Glu, Gly, His, Ile, Lys, Met, Asn, Pro, Gln, Arg, Ser, Thr or Val
  VARIANT (157)..(157): Glu, Cys, Asp, Gly, His, Asn, Pro or Gln
  VARIANT (158)..(158): Glu, Asp, Asn or Gln
  VARIANT (159)..(159): Gln, Arg, Asp, Glu or Asn
  VARIANT (161)..(161): His, Lys or Arg
  VARIANT (162)..(162): Ile, Ala, Gly, Leu or Val
XXXXXXXXXXXXXKXXGXXXXXXXXXXDXXXXXXXXXPXXXXXXXXXXXXXXXXXXXXXKXXXXXCXXKKXXXXXXXXPXXXXXXXLNEXKVLVLDXXYXXXXXXCXXXXXXPXXXXXCXCXXXXPXXXXXXXXKXXKXXKXXPXXXRXXXXPXXXXXXCXX

SEQ ID NO: 3 - protein, 123 aa, Bos taurus
  misc_feature: a-lactalbumin
  VARIANT (1)..(1): Glu, Asp, Asn or Gln
  VARIANT (2)..(2): Gln, Asp, Glu or Asn
  VARIANT (3)..(3): Leu, Gly, Ala, Val or Ile
  VARIANT (4)..(4): Thr, Ser or Met
  VARIANT (5)..(5): Lys, His or Arg
  VARIANT (7)..(7): Glu, Asp, Asn or Gln
  VARIANT (8)..(8): Val, Gly, Ala, Leu or Ile
  VARIANT (9)..(9): Phe, Tyr or Trp
  VARIANT (10)..(10): Arg, His or Lys
  VARIANT (11)..(11): Glu, Asp, Asn or Gln
  VARIANT (12)..(12): Leu, Gly, Ala, Val or Ile
  VARIANT (13)..(13): Lys, His or Arg
  VARIANT (14)..(14): Asp, Glu, Asn or Gln
  VARIANT (15)..(15): Leu, Gly, Ala, Val or Ile
  VARIANT (16)..(16): Lys, His or Arg
  VARIANT (17)..(17): Gly, Ala, Val, Leu or Ile
  VARIANT (18)..(18): Tyr, Phe or Trp
  VARIANT (19)..(19): Gly, Ala, Val, Leu or Ile
  VARIANT (20)..(20): Gly, Ala, Val, Leu or Ile
  VARIANT (21)..(21): Val, Gly, Ala, Leu or Ile
  VARIANT (22)..(22): Ser, Thr or Met
  VARIANT (23)..(23): Lue, Gly, Ala, Val or Ile
  VARIANT (25)..(25): Glu, Asp, Asn or Gln
  VARIANT (26)..(26): Trp, Phe or Tyr
  VARIANT (47)..(47): Ser, Thr or Met
  VARIANT (48)..(48): Thr, Ser or Met
  VARIANT (49)..(49): Glu, Asp, Asn, Gln
  VARIANT (50)..(50): Tyr, Phe or Trp
  VARIANT (51)..(51): Gly, Ala, Val, Leu or Ile
  VARIANT (52)..(52): Lue, Gly, Ala, Val or Ile
  VARIANT (53)..(53): Phe, Tyr or Trp
  VARIANT (54)..(54): Gln, Asp, Glu or Asn
  VARIANT (55)..(55): Ile, Gly, Ala, Val or Leu
  VARIANT (56)..(56): Asn, Asp, Glu or Gln
  VARIANT (57)..(57): Asn, Asp, Glu or Gln
  VARIANT (58)..(58): Lys, His or Arg
  VARIANT (93)..(93): Lys, His or Arg
  VARIANT (94)..(94): Lys, His or Arg
  VARIANT (95)..(95): Ile, Gly, Ala, Val or Leu
  VARIANT (96)..(96): Lue, Gly, Ala, Val or Ile
  VARIANT (97)..(97): Asp, Glu, Asn or Gln
  VARIANT (98)..(98): Lys, His or Arg
  VARIANT (99)..(99): Val, Gly, Ala, Leu or Ile
  VARIANT (100)..(100): Gly, Ala, Val, Leu or Ile
  VARIANT (101)..(101): Ile, Gly, Ala, Val or Leu
  VARIANT (102)..(102): Asn, Asp, Glu or Gln
  VARIANT (109)..(109): Ala, Gly, Val, Leu or Ile
  VARIANT (110)..(110): Leu, Gly, Ala, Val or Ile
  VARIANT (112)..(112): Ser, Thr or Met
  VARIANT (113)..(113): Glu, Asp, Asn or Gln
  VARIANT (114)..(114): Lys, His or Arg
  VARIANT (115)..(115): Leu, Gly, Ala, Val or Ile
  VARIANT (116)..(116): Asp, Glu or Asn
  VARIANT (117)..(117): Gln, Asp, Glu or Asn
  VARIANT (118)..(118): Trp, Phe or Tyr
  VARIANT (119)..(119): Leu, Gly, Ala, Val or Ile
  VARIANT (121)..(121): Glu, Asp, Asn or Gln
  VARIANT (122)..(122): Lys, His or Arg
  VARIANT (123)..(123): Leu, Gly, Ala, Val or Ile
XXXXXCXXXXXXXXXXXXXXXXXPXXVCTTFHTSGYDTQAIVQNNDXXXXXXXXXXXXIWCKDDQNPHSSNICNISCDKFLDDDLTDDIMCVXXXXXXXXXXYWLAHKXXCXXXXXXXXCXXX

SEQ ID NO: 4 - protein, 162 aa, Ovis aries musimon
  misc_feature: B-lactoglobulin
  VARIANT (6)..(6): Thr or Ser
  VARIANT (12)..(12): Ile or Leu
  VARIANT (18)..(18): Thr, Lys or Arg
  VARIANT (21)..(21): Ser or Asp
  VARIANT (29)..(29): Ile or Leu
  VARIANT (30)..(30): Ser, Asp or Lys
  VARIANT (36)..(36): Ser, Tyr, Leu, Lys, Met or Thr
  VARIANT (47)..(47): Lys, Leu or Pro
  VARIANT (47)..(52): LysProThrProGluGly or LeuProThrProGluAsp
  VARIANT (49)..(49): Thr or Lys
  VARIANT (59)..(59): Gln or Arg
  VARIANT (72)..(72): Ile, Leu, Phe, Trp or Tyr
  VARIANT (72)..(81): IleAlaGluLysThrLysIleProAlaVal or LeuGlyGluLysThrGluAsnProLysLys
  VARIANT (77)..(77): Lys or His
  VARIANT (78)..(78): Ile, Leu, Tyr, Trp or Phe
  VARIANT (83)..(90): LysIleAspAlaLeuAsnGluAsn or ThrValAsnTyrGlnGlyGluArg
  VARIANT (85)..(90): AspAlaLeuAsnGluAsn or AsnTyrLeuAspGluAsp
  VARIANT (86)..(86): Ala or Tyr
  VARIANT (90)..(90): Asn or Arg
  VARIANT (97)..(97): Thr or Ser
  VARIANT (102)..(102): Tyr or Phe
  VARIANT (108)..(118): GluAsnSerAlaGluProGluGlnSerLeuAla or GlyProProLeuProSerAlaGluHisGlyMetVal (wherein X116HisGly increases the protein length to 163 amino acids)
  VARIANT (110)..(110): Ser, Pro, Leu, Tyr, Trp, Phe or Lys
  VARIANT (111)..(111): Ala, Pro, Lys, Leu, Tyr, Trp or Phe
  VARIANT (125)..(125): Thr, Lys, Phe, Trp or Tyr
  VARIANT (127)..(127): Glu, Lys, Leu, Trp, Phe or Tyr
  VARIANT (127)..(130): GluValAspAsn or LysValAspLys
  VARIANT (128)..(128): Val or Leu
  VARIANT (137)..(137): Asp or Arg
  VARIANT (141)..(146): LysAlaLeuProMetHis or GlnProLeuProGlyArg
  VARIANT (142)..(142): Ala, Pro or Leu
  VARIANT (146)..(146): His or Arg
  VARIANT (151)..(156): PheAsnProThrGlnLeu or LeuAspLeuLysGlnMet
  VARIANT (153)..(155): ProThrGln or LeuThrArg
  VARIANT (154)..(154): Thr, Lys, Tyr, Leu, Trp or Phe
  VARIANT (159)..(159): Gln or Arg
IIVTQXMKGLDXQKVAGXWHXLAMAASDXXLLDAQXAPLRVYVEELXPXPEGNLEILLXKWENGECAQKKIXAEKTXXPAVFKIDXLNEXKVLVLDXDYKKXLLFCMENXXEPEQSLACQCLVRXPXXDNEALEKFXKALKXLPMXIRLAFNPXQLEGXCHV

SEQ ID NO: 5 - protein, 162 aa, Ovis aries
  misc_feature: B-lactoglobulin
  VARIANT (6)..(6): Thr or Ser
  VARIANT (12)..(12): Ile or Leu
  VARIANT (18)..(18): Thr, Lys or Arg
  VARIANT (21)..(21): Ser or Asp
  VARIANT (29)..(29): Ile or Leu
  VARIANT (30)..(30): Ser, Asp or Lys
  VARIANT (36)..(36): Ser, Tyr, Leu, Lys, Met or Thr
  VARIANT (47)..(47): Lys, Leu or Pro
  VARIANT (47)..(52): LysProThrProGluGly or LeuProThrProGluAsp
  VARIANT (49)..(49): Thr or Lys
  VARIANT (59)..(59): Gln or Arg
  VARIANT (72)..(72): Ile, Leu, Phe, Trp or Tyr
  VARIANT (72)..(81): IleAlaGluLysThrLysIleProAlaVal or LeuGlyGluLysThrGluAsnProLysLys
  VARIANT (77)..(77): Lys or His
  VARIANT (78)..(78): Ile, Leu, Tyr, Trp or Phe
  VARIANT (83)..(90): LysIleAspAlaLeuAsnGluAsn or ThrValAsnTyrGlnGlyGluArg
  VARIANT (85)..(90): AspAlaLeuAsnGluAsn or AsnTyrLeuAspGluAsp
  VARIANT (86)..(86): Ala or Tyr
  VARIANT (90)..(90): Asn or Arg
  VARIANT (97)..(97): Thr or Ser
  VARIANT (102)..(102): Tyr or Phe
  VARIANT (108)..(118): GluAsnSerAlaGluProGluGlnSerLeuAla or GlyProProLeuProSerAlaGluHisGlyMetVal (wherein X116HisGly increases the protein length to 163 amino acids)
  VARIANT (110)..(110): Ser, Pro, Leu, Tyr, Trp, Phe or Lys
  VARIANT (111)..(111): Ala, Pro, Lys, Leu, Tyr, Trp or Phe
  VARIANT (125)..(125): Thr, Lys, Phe, Trp or Tyr
  VARIANT (127)..(127): Glu, Lys, Leu, Trp, Phe or Tyr
  VARIANT (127)..(130): GluValAspAsn or LysValAspLys
  VARIANT (128)..(128): Val or Leu
  VARIANT (137)..(137): Asp or Arg
  VARIANT (141)..(146): LysAlaLeuProMetHis or GlnProLeuProGlyArg
  VARIANT (142)..(142): Ala, Pro or Leu
  VARIANT (146)..(146): His or Arg
  VARIANT (151)..(156): PheAsnProThrGlnLeu or LeuAspLeuLysGlnMet
  VARIANT (153)..(155): ProThrGln or LeuThrArg
  VARIANT (154)..(154): Thr, Lys, Tyr, Leu, Trp or Phe
  VARIANT (159)..(159): Gln or Arg
IIVTQXMKGLDXQKVAGXWHXLAMAASDXSLLDAQXAPLRVYVEELXPXPEGNLEILLXKWENGECAQKKIXAEKTXXPAVFKIDXLNEXKVLVLDXDYKKXLLFCMENXXEPEQSLACQCLVRXPXXDNEALEKFXKALKXLPMXIRLAFNPXQLEGXCHV

SEQ ID NO: 6 - protein, 162 aa, Equus caballus
  misc_feature: B-lactoglobulin
  VARIANT (6)..(6): Thr or Ser
  VARIANT (21)..(21): Ser or Asp
  VARIANT (29)..(29): Ile or Leu
  VARIANT (30)..(30): Ser, Asp or Lys
  VARIANT (36)..(36): Ser, Tyr, Leu, Lys, Met or Thr
  VARIANT (47)..(47): Arg, Leu or Pro
  VARIANT (49)..(49): Thr or Lys
  VARIANT (72)..(72): Phe, Leu, Trp or Tyr
  VARIANT (72)..(81): PheAlaGluLysThrGluSerProAlaGlu or LeuGlyGluLysThrGluAsnProLysLys
  VARIANT (78)..(78): Ser, Leu, Tyr, Trp or Phe
  VARIANT (90)..(90): Asp or Arg
  VARIANT (97)..(97): Thr or Ser
  VARIANT (102)..(102): Tyr or Phe
  VARIANT (108)..(117): LysASnAlaAlaThrProGlyGlnSerLeu or GlyProProLeuProSerAlaGluHisGlyMet (wherein X116HisGly increases the protein length to 163 amino acids)
  VARIANT (111)..(111): Ala, Pro, Lys, Leu, Tyr, Trp or Phe
  VARIANT (125)..(125): Thr, Lys, Phe, Trp or Tyr
  VARIANT (128)..(128): Val or Leu
  VARIANT (151)..(155): ProAspLeuThrArg or LeuASpLeuLysGln
  VARIANT (154)..(154): Thr, Lys, Tyr, Leu, Trp or Phe
TNIPQXMQDLDLQEVAGKWHXVAMAASDXXLLDSEXAPLRVYIEKLXPXPEDNLEIILREGENKGCAEKKIXAEKTEXPAEFKINYLDEXTVFALDXDYKNXLFLCMKNAXTPGQSLVCQYLARXQMXDEEIMEKFRRALQPLPGRVQIVPDLXRMAERCRI

SEQ ID NO: 7 - protein, 162 aa, Equus asinus
  misc_feature: B-lactoglobulin
  VARIANT (6)..(6): Thr or Ser
  VARIANT (21)..(21): Ser or Asp
  VARIANT (29)..(29): Ile or Leu
  VARIANT (30)..(30): Ser, Asp or Lys
  VARIANT (47)..(47): Arg, Leu or Pro
  VARIANT (49)..(49): Thr or Lys
  VARIANT (72)..(72): Phe, Leu, Trp or Tyr
  VARIANT (72)..(81): PheAlaGluLysThrGluSerProAlaGlu or LeuGlyGluLysThrGluAsnProLysLys
  VARIANT (78)..(78): Ser, Leu, Tyr, Trp or Phe
  VARIANT (90)..(90): Asp or Arg
  VARIANT (97)..(97): Thr or Ser
  VARIANT (102)..(102): Tyr or Phe
  VARIANT (108)..(117): LysASnAlaAlaThrProGlyGlnSerLeu or GlyProProLeuProSerAlaGluHisGlyMet (wherein X116HisGly increases the protein length to 163 amino acids)
  VARIANT (111)..(111): Ala, Pro, Lys, Leu, Tyr, Trp or Phe
  VARIANT (125)..(125): Thr, Lys, Phe, Trp or Tyr
  VARIANT (128)..(128): Val or Leu
  VARIANT (151)..(155): ProAspLeuThrArg or LeuASpLeuLysGln
  VARIANT (154)..(154): Thr, Lys, Tyr, Leu, Trp or Phe
TNIPQXMQDLDLQEVAGKWHXVAMAASDXXLLDSEEAPLRVYIEKLXPXPEDNLEIILREGENKGCAEKKIXAEKTEXPAEFKINYLDEXTVFALDXDYKNXLFLCMKNAXTPGQSLVCQYLARXQMXDEEIMEKFRRALQPLPGRVQIVPDLXRMAERCRI

SEQ ID NO: 8 - protein, 163 aa, Equus caballus
  misc_feature: B-lactoglobulin
  VARIANT (6)..(6): Thr or Ser
  VARIANT (21)..(21): Ser or Asp
  VARIANT (29)..(29): Ile or Leu
  VARIANT (30)..(30): Ser, Asp or Lys
  VARIANT (36)..(36): Ser, Tyr, Leu, Lys, Met or Thr
  VARIANT (47)..(47): Arg, Leu or Pro
  VARIANT (47)..(52): ArgProThrProGluGly or LeuProThrProGluAsp
  VARIANT (49)..(49): Thr or Lys
  VARIANT (72)..(72): Val, Leu, Phe, Trp or Tyr
  VARIANT (72)..(81): ValAlaGlnLysThrGluAspProAlaVal or LeuGlyGluLysThrGluAsnProLysLys
  VARIANT (78)..(78): Asp, Leu, Tyr, Trp or Phe
  VARIANT (97)..(97): Thr or Ser
  VARIANT (102)..(102): Tyr or Phe
  VARIANT (126)..(126): Thr, Lys, Phe, Trp or Tyr
  VARIANT (129)..(129): Val or Leu
  VARIANT (138)..(138): Ser or Arg
  VARIANT (152)..(157): GlnAspProSerGlyGly or LeuAspLeuLysGlnMet
  VARIANT (154)..(156): ProSerGly or LeuThrArg
TDIPQXMQDLDLQEVAGRWHXVAMVASDXXLLDSEXVPLRVYVEELXPXPEGNLEIILREGANHACVERNIXAQKTEXPAVFTVNYQGERKISVLDXDYAHXMFFCVGPPLPSAEHGMVCQYLARXQKXDEEVMEKFXRALQPLPGRVQIVQDPSGGQERCGF

SEQ ID NO: 9 - protein, 163 aa, Equus asinus
  misc_feature: B-lactoglobulin
  VARIANT (6)..(6): Thr or Ser
  VARIANT (21)..(21): Ser or Asp
  VARIANT (29)..(29): Ile or Leu
  VARIANT (30)..(30): Ser, Asp or Lys
  VARIANT (36)..(36): Ser, Tyr, Leu, Lys, Met or Thr
  VARIANT (47)..(47): Arg, Leu or Pro
  VARIANT (47)..(52): ArgProThrProGluGly or LeuProThrProGluAsp
  VARIANT (49)..(49): Thr or Lys
  VARIANT (72)..(72): Val, Leu, Phe, Trp or Tyr
  VARIANT (72)..(81): ValAlaGlnLysThrGluAspProAlaVal or LeuGlyGluLysThrGluAsnProLysLys
  VARIANT (78)..(78): Asp, Leu, Tyr, Trp or Phe
  VARIANT (97)..(97): Thr or Ser
  VARIANT (102)..(102): Tyr or Phe
  VARIANT (126)..(126): Thr, Lys, Phe, Trp or Tyr
  VARIANT (129)..(129): Val or Leu
  VARIANT (138)..(138): Ser or Arg
  VARIANT (147)..(147): His or Arg
  VARIANT (152)..(157): GlnAspProSerGlyGly or LeuAspLeuLysGlnMet
  VARIANT (154)..(156): ProSerGly or LeuThrArg
TDIPQXMQDLDLQEVAGRWHXVAMVASDXXLLDSEXAPLRVYVEELXPXPEGNLEIILREGANHVCVERNIXAQKTEXPAVFTVNYQGERKISVLDXDYAHXMFFCVGPCLPSAEHGMVCQYLARXQKXDEEVMEKFXRALQPLPGXVQIIQDPSGGQERCGF

SEQ ID NO: 10 - protein, 162 aa, Capra hircus
  misc_feature: B-lactoglobulin isoform B
  VARIANT (6)..(6): Thr or Ser
  VARIANT (12)..(12): Ile or Leu
  VARIANT (18)..(18): Thr, Lys or Arg
  VARIANT (20)..(20): Tyr, Ala, Cys, Asp, His, Asn, Ser or Thr
  VARIANT (21)..(21): Ser or Asp
  VARIANT (29)..(29): Ile or Leu
  VARIANT (30)..(30): Ser, Asp or Lys
  VARIANT (36)..(36): Ser, Tyr, Leu, Lys, Met or Thr
  VARIANT (47)..(47): Lys, Leu or Pro
  VARIANT (47)..(52): LysProThrProGluGly or LeuProThrProGluAsp
  VARIANT (49)..(49): Thr or Lys
  VARIANT (59)..(59): Gln or Arg
  VARIANT (72)..(72): Ile, Leu, Phe, Trp or Tyr
  VARIANT (72)..(81): IleAlaGluLysThrLysIleProAlaVal or LeuGlyGluLysThrGluAsnProLysLys
  VARIANT (77)..(77): Lys or His
  VARIANT (78)..(78): Ile, Leu, Tyr, Trp or Phe
  VARIANT (83)..(90): LysIleAspAlaLeuAsnGluAsn or ThrValAsnTyrGlnGlyGluArg
  VARIANT (85)..(90): AspAlaLeuAsnGluAsn or AsnTyrLeuAspGluAsp
  VARIANT (86)..(86): Ala or Tyr
  VARIANT (90)..(90): Asn or Arg
  VARIANT (97)..(97): Thr or Ser
  VARIANT (102)..(102): Tyr or Phe
  VARIANT (108)..(118): GluAsnSerAlaGluProGluGlnSerLeuAla or GlyProProLeuProSerAlaGluHisGyMetVal (wherein X116HisGly increases the protein length to 163 amino acids)
  VARIANT (110)..(110): Ser, Pro, Leu, Tyr, Trp, Phe or Lys
  VARIANT (111)..(111): Ala, Pro, Lys, Leu, Tyr, Trp or Phe
  VARIANT (125)..(125): Thr, Lys, Phe, Trp or Tyr
  VARIANT (127)..(127): Glu, Lys, Leu, Trp, Phe or Tyr
  VARIANT (128)..(128): Val or Leu
  VARIANT (137)..(137): Asp or Arg
  VARIANT (141)..(146): LysAlaLeuProMetHis or GlnProLeuProGlyArg
  VARIANT (142)..(142): Ala, Pro or Leu
  VARIANT (146)..(146): His or Arg
  VARIANT (151)..(156): PheAsnProThrGlnLeu or LeuAspLeuLysGlnMet
  VARIANT (153)..(155): ProThrGln or LeuThrArg
  VARIANT (154)..(154): Thr, Lys, Tyr, Leu, Trp or Phe
  VARIANT (159)..(159): Gln or Arg
IIVTQXMKGLDXQKVAGXWXXLAMAASDXXLLDAQXAPLRVYVEELXPXPEGNLEILLXKWENGECAQKKIXAEKTXXPAVFKIDXLNEXKVLVLDXDYKKXLLFCMENXXEPEQSLACQCLVRXPXXDKEALEKFXKALKXLPMXIRLAFNPXQLEGXCHV